SEQUENCE LISTING (179 sequences)

SEQ ID NO: 1 - protein, 214 aa, synthetic construct
DIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 222 aa, synthetic construct
EVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 214 aa, synthetic construct
DIVMTQSPSSLSASVGDRVTITCRASQDVGTAVAWYQQKPGQSPKLLIYWASTRYTGVPDRFSGSGSGTDFTLTISGLQSEDEADYFCQQYSNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 222 aa, synthetic construct
EVQLVESGGGLVKPGGSLKLSCAASGLTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - protein, 244 aa, synthetic construct
EVQLVESGGGLVKPGGSLKLSCAASGLTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSRLIEDICLPRWGCLWEDD

SEQ ID NO: 6 - protein, 244 aa, synthetic construct; identical to SEQ ID NO: 5

SEQ ID NO: 7 - protein, 20 aa, synthetic construct
QRLIEDICLPRWGCLWEDDF

SEQ ID NO: 8 - protein, 19 aa, synthetic construct
QRLMEDICLPRWGCLWEDD

SEQ ID NO: 9 - protein, 17 aa, synthetic construct
QRLMEDICLPRWGCLWE

SEQ ID NO: 10 - protein, 10 aa, synthetic construct
DICLPRWGCL

SEQ ID NO: 11 - protein, 125 aa, synthetic construct
QVQLVQSGGGLVQAGGSLRLSCAASGSSSSIIAMGWYRQAPGKQRELVATISNGDTTNYIDSVKGRFTISRDNAKNTVYLQMASLEPEDTAVYYCNARQTKWYAAGYKYRTYDYWGQGTQVTVSS

SEQ ID NO: 12 - protein, 127 aa, synthetic construct
QVQLVQSGGGLVQAGGSLRLSCAASERTFSTYAMGWFRQIPGKERLFVAAVNWNGKTIRYADSVKGRFTISRDNAKNTIALQMNSLKPEDTAVYYCALRSTPMYFTNLASQESYNYWGPGTQVTVSS

SEQ ID NO: 13 - protein, 125 aa, synthetic construct
QVQLVQSGGGLVQAGGSLRLSCAASGSSSSIIAMGWYRQAPGKQRELVATISNGDTTNYIDSVKGRFTISRDNAKNTVYLQMASLEPEDTAVYYCNARQTKWYAAGYEYRTYDYWGQGTQVTVSS

SEQ ID NO: 14 - protein, 125 aa, synthetic construct; identical to SEQ ID NO: 13

SEQ ID NO: 15 - protein, 125 aa, synthetic construct
EVQLVESGGGLVQPGGSLRLSCAASGRIFSISNMGWYRQAPGKQRELVATITSGGSTNYGDSVKGRFTISMANAKNAVYLQMNSLKPEDTAVYYCNAGISRRTGTYSGGRYSDYWAQGTQVTVSS

SEQ ID NO: 16 - protein, 125 aa, synthetic construct
QVQLVQSGGGLVQPGGSLRLSCAASGRVSEINTMGWYRQAPGKQRELVALITSTSSTMYSDSVKGRFTVSRDVAKNMVYLQMNSLKPEDTAVYYCNARQTKWYAAGYEYRTYDYWGQGTQVTVSS

SEQ ID NO: 17 - protein, 127 aa, synthetic construct
QVQLQESGGGLVQAGGSLRLSCAASERTFSTYAMGWFRQTPGKERLFVAAVNWNGKTIRYADSVKGRFTISRDNAKNTMSLQMNSLKPEDTAVYYCALRSTPMYFTNLASQESYNYWGPGTQVTVSS

SEQ ID NO: 18 - protein, 125 aa, synthetic construct
QVQLVQSGGGLVQPGGSLRLSCAASGRIFSISNMGWYRQAPGKQRELVATITSGGSTNYGDSVKGRFTISMVNAKNAVYLQMNSLKPEDTAVYYCNAGISRRTGTYSGGRYSDYWGQGTQVTVSS

SEQ ID NO: 19 - protein, 125 aa, synthetic construct
QVQLVQSGGGLVQAGGSLRLSCAASGSSSSIIAMGWYRQAPGKQRELVATISNGDTTNYTDSVKGRFTISRDKAKNTVYLQMASLEPEDTAVYYCNARQTKWYAAGYVYRTYDYWGQGTQVTVSS

SEQ ID NO: 20 - protein, 125 aa, synthetic construct
QVQLVQSGGGLVQAGGSLRLSCAASGSSSSIIAMGWYRQAPGKQRELVATISNGDTTNYIDSVNGRFTISRDNAKNTVYLQMTSLEPEDTAVYYCHARQTKWYAAGYKYRTYDYWGQGTQVTVSS

SEQ ID NO: 21 - protein, 125 aa, synthetic construct
QVQLVQSGGGLVQPGGSLRLSCAASGRVSEINTMGWYRLAPGKQRELVALITSTSSTMYSDSVKGRFTVSRDVAKNMVYLQMNSLKPEDTAVYYCNARQTKWYAAGYKYRTYDYWGQGTQVTVSS

SEQ ID NO: 22 - protein, 125 aa, synthetic construct
QVQLVQSGGGLVQPGGSLRLSCAASGRVSEINTMGWYRQAPGKQRELVALITSTSSTMYSDSVKGRFTVSRDVAKNMVYLQMNSLKPEDTAVYYCNARQTIWYAAGYKYRTYDYWGLGTQVTVSS

SEQ ID NO: 23 - protein, 125 aa, synthetic construct
QVQLVQSGGGLVQSGGSLRLSCAASGSSSSIIAMGWYRQAPGKQRELVATISNGDTTNYIDSVKGRFTISRDNAKNTVYLQMASLEPEDTAVYYCNARQTKWYAAGYEYRTYDYWGQGTQVTVSS

SEQ ID NO: 24 - protein, 125 aa, synthetic construct
QVQLVQSGGGLVQAGGSLRLSCAASGSSSSIIAMGWYRQAPGKQRELVATISNNDTTNYIDSVKGRFTISRDNAKNTVYLQMASLEPEDTAVYYCNARQTKWYAAGYKYRTYDYWGQGTQVTVSS

SEQ ID NO: 25 - protein, 125 aa, synthetic construct
QVQLVQSGGGQVQAGGSLRLSCAASGSSSSIIAMGWYRQAPGKQRELVATISNGDTTNYIDSVKGRFTISRDNAKNTVYLQMASLEPEDTAVYYCNARQTKWYAAGYKYRTYDYWGQGTQVTVSS

SEQ ID NO: 26 - protein, 125 aa, synthetic construct
QVQLVQSGGGLAQAGGSLRLSCAASGSSSSIIAMGWYRQAPGKQRELVATISNGDTTNYIDSVKGRFTISRDNAKNTVYLQMASLEPEDTAVYYCNARQTKWYAAGYKYRTYDYWGQGTQVTVSS

SEQ ID NO: 27 - protein, 125 aa, synthetic construct
QVQLVQSGGGLVQPGGSLRLSCAASGRVSEINTMGWFRQAPGKQRELVALITSTSSTMYSDSVKGRFTVSRDVAKNMVYLQMNSLKPEDTAVYYCNARQTKWYAAGYKYRTYDYWGQGTQVTVSS

SEQ ID NO: 28 - protein, 125 aa, synthetic construct
QVQLVQSGGGLVQAGGSLRLSCAASGSSSSIIAMGWYRQAPGKQRELVATISNGDTTNYIDSVKGRFTISRDNAKNTVYLQMASLEPEDTAVYYCNARQTRWYAAGYKYRTYDYWGQGTQVTVSS

SEQ ID NO: 29 - protein, 125 aa, synthetic construct
QVQLVQSGGGLVQPGGSLRLSCAASGRVSEINTVGWYRQAPGKQRELVALFTSTSSTMYSDSVKGRFTVSRDVAKNMVYLQMNSLRPEDTAVYYCNARQTKWYAAGYKYRTYDYWGQGTQVTVSS

SEQ ID NO: 30 - protein, 125 aa, synthetic construct
QVQLVQSGGGLVQAGGSLRLSCAASGSSSSIIAMGWYRQAPGKQRELVATISNGDTTNYIDSVKGRFTISRDNAKNTVYLRMASLEPEDTAVYYCNARQTKWYAAGYKYRTYDYWGQGTQVTVSS

SEQ ID NO: 31 - protein, 247 aa, synthetic construct
DIVMTQSHKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSEVKIEESGGGLVKAGGSLKLSCAASGFTFSRYDMSWVRQTPEKRLEWVATISSGGRHTYYPDSVKGRFTISRDNVKNTLYLQMNSLRSVDTAMFYCLASMLTTDYFEYWGQGTSLTVSS

SEQ ID NO: 32 - protein, 247 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIKRGGGGSGGGGSGGGGSGGGGSEVKLVESGGGLVKPGGSLKLSCAASGFTFNRYALSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 33 - protein, 246 aa, synthetic construct
DVVMTQTQKFMSTSVGDRVSITCKASQNVGTAVAWYQQKPGQSPKLLIYSASNRYTGVPDRFTGSGSGTDFTLTISNMQSEDLADYFCQQYSSYPYTSGGGTKLEIKRGGGGSGGGGSGGGGSSGGGSQVQLVETGGGLVKPGGSLKLSCAASGFTFSGYAMSWFRQTPEKRLEWVATISSGGSYTYYPDSVKGRFTSSRDNAKNTLYLQMSSLRSEDTAMYYCLASLITTDYFEYWGQGTTVTVL

SEQ ID NO: 34 - protein, 248 aa, synthetic construct
MDIVMTQSHKFMSTSVGDRVSITCKASQDVDTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSEVKLVESGGGLVKPGGSLKLSCAASGFTFNRYALSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 35 - protein, 248 aa, synthetic construct
MDVVMTQTQKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRCGGGSGGGGSGGGGSGGGGSQVQLVETGGGLVKPGGSLKLSCAASGFTFSGYAMSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 36 - protein, 248 aa, synthetic construct
MDVVMTQTQKFMSTSVGDRVSITCKASQDVGTAVAWYQQKPGQSPKLLIYWASTRHTGVPVRFTGSGSGTDFTLTISNVQSEDLADYFCQQYSNYPYTFGGGTKLEIKRGGGGSGGGGSGGGGSGGGGSQVQLVETGGGLVKPGGSLKLSCAASSSTFSGYAMSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 37 - protein, 248 aa, synthetic construct; identical to SEQ ID NO: 34

SEQ ID NO: 38 - protein, 248 aa, synthetic construct
MDIVMTQSQKFMSTSVGDRASVTCKASQDVGTAVAWYQQKPGQSPKLLIYWASTRHTGVPDRFTGSGSGTDFTLTISEVQSEDLADYFCQQYSNYPYTFGGGTKLEIKRGGGGSGGGGSGGGGSGGGGSEVKLVESGGGLVKPGGSLKLSCAASGFTFNRYALSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 39 - protein, 248 aa, synthetic construct
MDIVMTQSHKFMSTSVGDRVSITCKASQDVGTAVAWYQQKPGQSPKLLIYWASTPHTGVPDRFTGSGSGTDFTLTISNVQSEDLAEYFCQQYNSYPYTFGGGTKLEIKRGGGGSGGGGSGGGDSGGGGSEVKLVESGGGLVIPGGSLKLSCAASGFTFNRYALSWFRQTPEKRLEWVATISSGGSYTYYLDSVKGRFTVSRDIAKNTLYLQMSSLRSEDTAMYYCLASLFTTDYFEYWGQGTTVTVPL

SEQ ID NO: 40 - protein, 248 aa, synthetic construct
MDIVMTQSHKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSEVKLVESGGGLVKPGGSLKLSCAARGFTFNRYALSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 41 - protein, 248 aa, synthetic construct
MDIVMTQSQKFMSTSVGDRVSITCKASQDVGTAVAWYQQKPGQSPKLQIYWASTRHTGVPDRFTGSGSGTDFTLTISNVQSEDLADYFCQQYSNYPYTFGGGTKLEIKRGGGGSGGGGSGGGGSGGGGSEVKLVESGGGLVKPGGSLKLSCAASGFTFNRYALSWFRQTPENRLEWVATISSGGSYTYYLDSVKGRFTVSRDNAKNTLYLQMSSLRSEDTAMYYCLASLITTDYFEYWGQGTTVTVLL

SEQ ID NO: 42 - protein, 248 aa, synthetic construct
MDIVMTQSHKFMSTSIGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSQVQLVETGGGLVKPGGSLKLSCAASGFTFSGYAMSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 43 - protein, 248 aa, synthetic construct
MDIVMTQSHKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSEVKLVESGGGLVKPGGSLKLSCAASGFTLNRYALSWFRQTPEKRLEWVATISSGGSYTYYLDSVKGRFTVSRDNAKNTLYLQMSSLRNEDTAMYYCLASLITTDYFEYWGQGTTVTVLL

SEQ ID NO: 44 - protein, 248 aa, synthetic construct
MDIVMTQSHKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSEVKIEESGGGLVKAGGSLKLSCAASGFTFNRYALSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 45 - protein, 248 aa, synthetic construct
MDVVMTQTQKFMSTSVGDRVSITCKASQNVGTAVAWYQQKPGQSPKLLIYSASNRYTGVPDRFTGSGSGTDFTLTISNVQSEDLADYFCQQYSNYPYTFGGGTKLEIKRGGGGSGGGGSGGGGSGGGGSEVKLVESGGGLVKPGGSLKLSCAASGFTFNRYALSWFRQTPENRLEWVATISSGGSYTYYPDSVKGRFTSSRDNAKNTLYLQMSSLRSEDTAMYYCLASLITTDYFEYWGQGTTVTVLL

SEQ ID NO: 46 - protein, 248 aa, synthetic construct
MDIVMTQSHKFVSTSVGDRVSITCKASQDVGTAVAWYQQKPGQSPKLLIYWASTLHTGVPDRFTGSGSGTDFTLTISNVQNEDLADYFCQQYSNYPYTFGGGTKLEIKRGGGGSGGGGSGGGGSGGGGSEVKLVESGGGLVKPGGSLKLSCAASGFTFNRYALSWFRQTPEKRLEWVATISSGGSYTYYLDSVKGRFTVSRDNAKNTLYLQMSSLRSEDTAMYYCLASLITTDYSEYWGQGTTVTVLP

SEQ ID NO: 47 - protein, 248 aa, synthetic construct
MDVVMTQTQKFMSTSVGDRVSITCKASQNVGTAVAWYQQKPGQSPKLLIYSASNRYTGVPDRYTGSGSGTDFTLTISNMQSEDLADYFCQQYSNYPYTFGGGTKLEIKRGGGGSGGGGSGGGGSGGGGSEVKLVESGGGLVKPGGSLKLSCAASGFTFNRYALSWFRQTPEKRLEWVATISSGGSYTFYLDSVKGRFTVSRDNAKNTLYLQMSSLRSEDTAMYYCLASMLTTDYFEYWGQGTTVTVLL

SEQ ID NO: 48 - protein, 248 aa, synthetic construct
MDIVMTQSHKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSEVKLVESGGGLVKPGGSLKLSCAASGFTFNRYALSWFRQTPEKRLEWVATISSGGSYTYYLDSVKGRFTVSRDNAKNTLYLQMSSLRSEDTAMYYCLASLITTDYFEYWGQGTTVTVLL

SEQ ID NO: 49 - protein, 248 aa, synthetic construct
MDIVMTQSHKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSQVQLVETGGGLVKPGGSLKLSCAASGFTFSGYAMSWFRQTPEKRLEWVATISSGGSYSYYLDSVKGRFTVSRDNAKNTLYLQMSSLRSEDTAMYYCLASLITTDYFEYWGQGTTVTVLL

SEQ ID NO: 50 - protein, 248 aa, synthetic construct
MDVVMTQTQKFMSTSVGDRVSITCKASQDVGTAVAWYQQKPGQSPKLLIYWASTRHTGVPDRFTGSGSGTDFTLTISNVQSEDLADYFCQQYSNYPYTFGGGTKLEIKRGGGGSGGGGSGGGGSGGGGSQVQLVETGGGLVKPGGSLKLSCAASGFTFSGYAMSWFRQTPEKRLEWVATISSGGSYTYYLDSVKGRFTVSRDNAKNTLYLQMSSLRSEDTAKYYCLASLFTTDYFEYWGQGTTVTVLL

SEQ ID NO: 51 - protein, 240 aa, synthetic construct
EVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSDICLPRWGCLWEDD

SEQ ID NO: 52 - protein, 238 aa, synthetic construct
EVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSDICLPRWGCLWE

SEQ ID NO: 53 - protein, 222 aa, synthetic construct
EVQLVESGGGLVKPGGSLKLSCAASGFTFSGYAMSWFRQAPGKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 54 - protein, 222 aa, synthetic construct
EVQLVESGGGLVKPGGSLKLSCAASGFTFSGYAMSWFRQAPEKGLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 222 aa, synthetic construct
EVQLVESGGGLVKPGGSLKLSCAASGFTFSGYAMSWFRQAPEKRLEWVATISSGGSYTYYADSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 222 aa, synthetic construct
EVQLVESGGGLVKPGGSLKLSCAASGFTFSGYAMSWVRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 248 aa, synthetic construct
MDIVMTQSPSSLSASVGDRVTITCRASQDVGTAVAWYQQKPGQSPKLLIYRASTRHTGVPDRFSGNGSGTDLTLTISGLQSEDEADYFCQQYSNYPYTFGGGTKLEIKRGGGGSGGGGSGGGGSGGGGSEVQLVESGGGLVKPGGSLKLSCAASGFSFSGYAMSWFRQAPDKRLEWVATISSGGSYTYYLDSVKGRFTISRDNSKNTLYLQMSSLRSEDTAVYYCLASLITTDYFEYWGQGTLVTVSS

SEQ ID NO: 58 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRPEWVATISSGGSYTYYLDSVKGRFTISRDNNKNTLFLQMSSLRSEDTAVYYCLASMITTDYFEYWGQGTLVTVSS

SEQ ID NO: 59 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTELEIKRGGSGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLVTTDYFEYWGQGTLVTVSS

SEQ ID NO: 60 - protein, 248 aa, synthetic construct
MDIVMTQSTSSMRASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 61 - protein, 248 aa, synthetic construct
MDIVMTQSPSSLSASVGDRVIFTCRTSQDVGTAVAWYQQKPGQSPKLLIYWASTRHTGVPDRFSGSGSGTDFTLTISGLQSEDEADYFCQQYSNYPYTFGGGTELEVKRSGGGSGGGGSGGGGSGGGGSEVQLVESGGGLVKPGGSLKLSCAASGFTFSGYAMSWFRQAPEKRLEWVATISSSGSYTYYLDSVKGRFTISRDNSKNTLYLQMSSLRSEDTAVYYCLASLMTTDYFEYWGQGTLVTVSS

SEQ ID NO: 62 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQFSNYPYTFGGGTKLEIKRGGGGSGGGGSGGGGSGGGGSEVQLVESGGGLVKPGGSLKLSCAASGLTFSGYAMSWFRQAPEKRLEWVATISSGGSYTYYLDSVKGRFTISRDNSKKTLYLQMSSLRSEDTAVYYCLASLITTDYFEYWGQGTLVTVSS

SEQ ID NO: 63 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSGVQLVESGGGLVKPGGSLKLSCAASGFTISGYAMSWFRQTPEKRLEWVATISSGGSYTYYLDSVKGRFTTSRDNSINTLYLQMSSLRSEDTAVYYCLASLFTTDYFEYWGQGTLVTVNS

SEQ ID NO: 64 - protein, 248 aa, synthetic construct
MDIVMTQSPSSLSASVGDRVTISCRASQDVGTAVAWYQQKPGQSPKLLIYWASTRHTGVPDRFSGSGSGTDFTLTISGLQSEDEADYFCQQYSNYPYTFGGGSKLEIKRGGGGSGGGGSGGGGSGGGGSEVQLVESGGGLVKPGGSLKLSCAASGFTFSGYAMSWFRQTPEKRLEWVATISSGGSYTYYLDSVKGRFTISRDNSKNTLYLQMSSLRSEDTAVYYCLASLITTDYFEYWGQGTLVTVSS

SEQ ID NO: 65 - protein, 248 aa, synthetic construct
MDIVMTQSPSSLSASVGDRVTITCRASQDVGTAVAWYQQKPGQSPKLLIYWASTRHTGVQDRFSGSGSGTVFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPGKRLEWVATISSGGSYTYYLDSVKGRFTISRDNSKNTLYLQMSSLRSEDTAVYYCLASLITTDYFEYWGQGTLVTVSS

SEQ ID NO: 66 - protein, 248 aa, synthetic construct
MDIVMTQSPSSLGASVGDRVTITCRASQDIGTAVAWYQQKPGQSPKLLIYWASTRHTGVPDRFSGSGSGTDFTLTISGLQSEDEADYFCQQYSNYPYTFGAGTKLEIRRGGGGSGGGGSGGGGSGGGGSEVQLDESGGGLVKPGGSLKLSCAASGFTFSGYAMSWFRQAPDKRLEWVATISSGGSYTYYLDSVKGRFTISRDNSKNTLYLQMSSLRSEDTAVYYCLASLITTDYFEYWGQGTLVTVSS

SEQ ID NO: 67 - protein, 248 aa, synthetic construct
MDIVMTQSPSSLSTSVGDRVTITCRASQDVGTAVAWYQQKPGQSPKLLIYWASTRHTGVPGRFSGSGSGTDFTLTISGLQSEDEADYFCQQYSNYPYTFGGGTKLEINRGGGGSGGGGSGGGGSGGGGSEVQLVESGGGLVKPGGSLKLSCAASGFTFSGYAMSWFRQAPEKRLEWVATISSGGSYTYYLDSVKGRFTVSRDNSKNTLYLQMSSLRSEDTAVYYCLASLITTDYFEYWGQGTLVTVSS

SEQ ID NO: 68 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVERGGSLKLSCAASGFTFSGYAMSWFRQAPEKRLEWVATISSGGSYTYYLDSVKGRFTISRDNSKNTLYLQMSSLRSEDTAVYYCLASLLTTDYFEYWGQGTLVTVSS

SEQ ID NO: 69 - protein, 248 aa, synthetic construct
MDIVMTQSPSSLNASVGGRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGLTFSGYAMSWFRQAPEKRLEWVGTISSGGSYTYYLDSVKGRFTISRDNSKNTLYLQMSSLRSEDTAVYYCLASLITTDYFEYWGQGTLVTVSS

SEQ ID NO: 70 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYLEYWGQGTLVTVSS

SEQ ID NO: 71 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTHYLDSVKGRFTISRDNSKNTLYLQMSSLRSEDTAVYYCLASLITTDYFEYWGQGTLVTVSS

SEQ ID NO: 72 - protein, 248 aa, synthetic construct
MDIVMTQSPSSQSASIGDRVTITCRASQDVGTAVAWYQQKPGQSPKLLIYWASTRHTGVPDRFSGSGSGTDFTLTISGLQSEDEADYFCQQYSNYPYTFGGGTKLEIERGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQTSSLRSEDTAVYYCLASLITTDYFEYWGQGTLVTVSS

SEQ ID NO: 73 - protein, 248 aa, synthetic construct
MDIVMTQSPSGLSASVGDRVTITCHASQDVGTAVAWYQQKPGQSPKLLIYWASTRHTGVPDRFSGSGSGSDFTLTISGLQSEDEADYFCQQYSNYPYTFGGGTKLEIKRGGGGSGGGGSGGGGSGGGGSEVQLFESGGGLVKPGGSLKLSCAASGFTFSGYAMSWFRQAPEKRPEWVATISSGGSYTYYLDSVKGRFTISRDNSKNTLYLQMSSLRSEDTAVYYCLASLITTDYFEYWGQGTLVTVSS

SEQ ID NO: 74 - protein, 248 aa, synthetic construct
MDIVMTQSPSSLSASVGDRVTITCSASQDVGTAGAWYQQKPGQSPKLLIYWASTRHTGVPDRFSGSGSGTDFTLTISGLQSEDEADYFCQQYSNYPYTFGGGTKLEIKRGGGGSGGGGSGGGDSGSGGSEVQLVESGGGLVKRGGSLKLSCAASGFTLSGYAMSWFRQAPGKRPEWVATISSGGSYTYYLDSVKGRFTISRDNSKNTLYLQMSSLRSEDTAVYYCLASLITTDYFEYWGQGTLVTVSS

SEQ ID NO: 75 - protein, 247 aa, synthetic construct
MDIVMTQSPSSLSASVGDRVTFTCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVS

SEQ ID NO: 76 - protein, 248 aa, synthetic construct
MDIVMTQSPSSLSASVGDRVTITCHASQDVGTAVAWYQQKPGQSPKLLIYWASTRHTGVPDRFSGSGSGTDFNLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPDKRLEWVATISSGGGYTYYLDSVKGRFTISRDNSKNTLYLQMSSLRSEDTAVYYCLASLITTDYFEYWGQGALVTVSS

SEQ ID NO: 77 - protein, 248 aa, synthetic construct
MDIVMTQSPSSLSASVGDRVTITCRASQDVGTAVAWYQQKPGQSPKLLIYWASTRYTGVPDRFSGSGSGTDFTLTISGLQSEDEADYFCQQYSNYPYTFGGGTKLEIKRGGGGSGGGGSGGGGSGGGGSKVQLVESGGGLVKRGGSLKLSCAASGFTFSGYAMSWFRQAPEKRLEWVATISSGGSYTYYLDSVKGRFTISRDNSKNTLYLQLSSLRSEDTAVYYCLASLITTDYFEYWGQGTLVTVSS

SEQ ID NO: 78 - protein, 248 aa, synthetic construct
MDIVMTQSPSSLSASVGDRVTITCRASQDVGTAVAWYQQKPGQSPKLLIYWASTRYTGVPDRFSGSGSGTDFTLTISGLQSEDEADYFCQQYSNYPYTFGGGTKLEIKRGGGGGGGGGSGGGGSGGGGSEVQLVESGGGLVKPGGSLKLSCAASGLTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 79 - protein, 248 aa, synthetic construct
MDIVMTQSPSSLSASVGDRVTITCRTSQDVGTAVAWYQQKPGQSPKLLIYWASTRHTGVPDRFSGSGSGTDFTLTISGLQSEDEADYFCQQYSNYPYTFGAGTKLEIKRGGGGSGGGGSGGGGSGGGGSEVQLVESGGGLVKPGGSLKLSCAASGLTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 80 - protein, 248 aa, synthetic construct
MDIVMTQSPSSLSASVGDRVTITCRASQDVGTAVAWYRQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKGGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 81 - protein, 248 aa, synthetic construct
MDIVMTQSPSSLSASVGDRVTIACRASQDVGTAVAWYQQKPGQSPKLLIYWASTRHTGVPDRFSGSGSGTDFTLTISGLQSEDEADYFCQQYSNYPYTFGGGTKLEVKRGGGGSGGGGSGSGGSGGGGSEVQLVESGGGLVKPGGSLKLSCAASGSTFSGYAMSWFRQAPDKRLEWVATISSGGSYTYYLDSVKGRYTISRDNSKNTLYLQMSSLRSEDTAVYYCLASRITTDYFEYWGQGTLVTVSS

SEQ ID NO: 82 - protein, 248 aa, synthetic construct
MDIVMTQSPSSLSASVGDRVTITCRASQDVGAAVAWYKKKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 83 - protein, 248 aa, synthetic construct
MDIVMTQSPSSLSASVGDRVTITCHASQDVGTAVAWYQQKPGQSPKLLIYWASTRHTGVPDRFSGSGSGTDFTLTISGLQSEDEADYSCQQYSNYPYTFGGGTKLEIKRGGGGSGGGGSGGGGSGGGGSEVQLVESGGGRVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 84 - protein, 248 aa, synthetic construct
MDIVMTQSPSSLSASVGDRVTITCHASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPDKRLEWVATISSGGSYTYYLDSVKGRFTISRDNSKNTLYLQMSSLRSEDTAVYYCLASLITTDYFEYWGQGTLVTVSS

SEQ ID NO: 85 - protein, 248 aa, synthetic construct
MDIVMTQSPSSLSASVGDRVTITCRASQDVGTAVAWYQQKPGQSPKLLIYWASTRHTGVPGRFSGSGSGTDFTLTIRGLQSEDEADYFCQQYSNYPYTFGGGTKLDIKRGGGGSGGGGSGGGGSGGGGSEVQLVESGGGLVKPGGSLKLSCAASGFTFSGYAMSWFRQAPDKRLEWVATISSGGSYTYYLDSVKGRFTISRDNSKNTLYLQMSSLRSEDTAVYYCLASLFTTDYFEYWGQGTLVTVSS

SEQ ID NO: 86 - protein, 248 aa, synthetic construct
MDIVMTQSPSSLSASVGDRVTITCRASQDVGTAVAWYQQKPGQSPKLLIHWASTRHTGVPDRFSGSGSGTDFTLTISGLQSEDEADYFCQQYSNYPYTFGGGTKMEIKRGGGGSGGGGSGGGGSGGGGSEVQLVESGGGLVKPGGSLKLSCAASGFTFSGYAMSWFRQAPGKRLEWVATISSGGSYTYYLDSVKGRFTISRDNSKNTLYLQMSSLRSEDTAVYYCLASLITTDYFEYWGQGTLVTVSS

SEQ ID NO: 87 - protein, 248 aa, synthetic construct
MDIVMTQSPSSLSASVGGRVTITCRASQDVGTAVAWYQQKPGQSPKLLIYWASTRYTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGLTFSGYAMSWFRQAPEKRLEWVATISSGGSYTYYLDSVKGRLTISRDNSKNTLYLQMSSLRSEDTAVYYCLASLITTDYFEYWGQGTLVTVSS

SEQ ID NO: 88 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 89 - protein, 248 aa, synthetic construct
MDIVMTQSPSSLSASVGDRVTITCRASQDVGTAVAWYQQKPGQSPKLLIYWASTRHTGVPDRFSGSGSGADFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMNSLRSEDTAVYYCLASLITTDYFEYWGQGTLVTVSS

SEQ ID NO: 90 - protein, 248 aa, synthetic construct
MDIVMTQSPGSLSASVGDRVTITCRASQDVGTAVAWYQQKPGQSPKLLIYWASTRHTGVPDRLSGSGSGTDFTLTISGLQSEDEADYFCQQYSNYPYTFGGGTKLEIKRGGGGSGGGGSGGGGSGGGGSEVQLVESGGGLVKPGGSLKLSCAASGSTFSGYAMSWFRQAPEKRPEWVATISSGGSYTYYLDSVKGRFTISRDNSKNTLYLQMSSLRSEDTAVYYCLASLITTDYFEYWGQGTLVTVSS

SEQ ID NO: 91 - protein, 248 aa, synthetic construct
MDIVMTQSPSSLRASVGGRVTITCRASQDVGTAVAWYQQKPGQSPKLLIHWASTRHTGVPDRFSGSGSGTDFTLTISGLQSEDEADYFCQQYSNYPYTFGGSTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSINTLYLQMSSLRSEDTAVYYCLASQFTTDYFEYWGQGNLVTVSS

SEQ ID NO: 92 - protein, 248 aa, synthetic construct
MDNVMTQSPSSLSASVGDRVTITCHASQDVGTAVAWYQQKPGRSPKLLIYWASTRHTGVPDRFSGSGSGTDFTLTISGLQSVDGADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 93 - protein, 248 aa, synthetic construct
MDIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEINRGGGGSGGGGSGGGGSGGGGSEVQLVESGGGLVKPGGSLKLSCAASGFTLSGYAMSWFRQAREKRLEWVATISSGGSYTYYLDSVKGRFTISRDNSKNTLYLQMSSLRSEDTAVYYCLASLITTDYFEYWGQGTPVTVSS

SEQ ID NO: 94 - protein, 248 aa, synthetic construct
MDIVMTQSPSSLSASVGDRVTITCRASQDVGTAVAWYQQKPGQSPKLLTYWASTRHTGVPDRFSGSGSGTDFTLTISGLQSEDEADYSCQQYSNYPYTFGGGTKLEIKRGGGGSGGGGSDGGGTGGGGSEVQLVESGGGLIKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSS

SEQ ID NO: 95 - protein, 248 aa, synthetic construct
MDIVMTQSLSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRGGGGSGGGGSGGGGSGGGGSEVQLVESGGGLVKPGGSLKLSCAASGFTFSGYAMSWFRQAPEKRLEWVATISSGGNYTYYLDSVKGRFTISRDNSKNILYLQMSSLRSEDTAVYYCLASLITTDYFEYWGQGTLVTVSS

SEQ ID NO: 96 - protein, 248 aa, synthetic construct
MDIVMTQSPSSLSASVGDRVTITCRASQDVGTAVAWYQQNPGQSPKLLIYWASTRHTGVPDRFSGSGSGTDFTLTISSLQSEDEADYFCQQYSNYPYTFGGGTKLEIKRGGGGSGGGGSGGGGSGGGGSEVQLVESGGGLVKPGGSLKLSCAASGFTFSGYTMSWFRQAPEKRLEWVATISSGGSYTYYLDSVKGRFTISRDNSKKTLYLQMSSLRSEDTAVYYCLASLITTDYFEYWGQGTLVTVSS

SEQ ID NO: 97 - protein, 214 aa, synthetic construct; identical to SEQ ID NO: 1

SEQ ID NO: 98 - protein, 244 aa, synthetic construct
EVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSRLIEDICLPRWGCLWEDD

SEQ ID NO: 99 - protein, 111 aa, synthetic construct
QAVLTQPSSVSGAPGQRVTISCTGSSSNIGAYYVHWYQQVPGTAPRLLIFDNDNRPSGVPDRFSASKSGTSASLAIIGLQAEDEAEYYCQSVDYSLGDGVVFGGGTKLTVL

SEQ ID NO: 100 - protein, 113 aa, synthetic construct
QVQLVESGPGLVKPSETLSLTCAVSGGSISSNNWWSWVRQTPGKGLEWIGEIYHSGSTNYNPSLKSRVTISVDKSKNQFSLKLSSVTAADTAVYYCANLRGQWGQGTLVTVSS

SEQ ID NO: 101 - protein, 112 aa, synthetic construct
QSVVTQPPSVSGAPGQRVTISCTGSSSNIGAGYDVHWYQQLPGTAPRLLISGNTNRPSGVPDRFSGSKSGTSASLAITGLQAEDEADYYCQSYDSSLSGHVVFGGGTKLTVL

SEQ ID NO: 102 - protein, 116 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGKRGNFDYWGQGTLVTVSS

SEQ ID NO: 103 - protein, 112 aa, synthetic construct
QSVLTQPPSVSGAPGQRVTISCTGSSSNIGAGYDVHWYQQLPGTAPKLLIYDNINRPSGVPDRFSGSKSGASASLAITGLQAEDEADYYCQSYDSSLSGLRVFGTGTKVTVL

SEQ ID NO: 104 - protein, 116 aa, synthetic construct
QVQLQESGPGLVEPSGTLSLTCAVSGVSISSRNWWSWVRQTPGKGLEWIGEINHSGSTNYNPSLKSRVTISVDTSKNQFSLKLSSVTAADTAVYYCARGGSDMDVWGKGTMVTVSS

SEQ ID NO: 105 - protein, 117 aa, synthetic construct
QLQLQESGPGLVKPSETLSLTCTVSGGSISSGNYWSWVRQSPEKGLEWIGEISHSGITNYNPSLQSRVTIALDKSKNHFSLNVNSVTAADTAVYYCVGTLRTWFDYWGQGTLVTVSS

SEQ ID NO: 106 - protein, 112 aa, synthetic construct
QAVLTQPSSVSGAPGQRVTISCTGSSTNIGAGFDVHWYQQLPGTAPKLLIYGDKNRPSGVPDRFSGSKSGTSAYLAITGLQAEDEADYYCQTYDSRLSGSKVFGGGTKVTVL

SEQ ID NO: 107 - protein, 114 aa, synthetic construct
QLQLQESGPGLVKPSGTLSLTCAVSGVSISSRNWWSWVRQTPGKGLEWIGEISHSGSTNYNPSLKSRVTISVDTSKNQFSLKLSSVTAADTAVYYCARNAGDIWGQGTMVTVSS

SEQ ID NO: 108 - protein, 110 aa, synthetic construct
QSVLTQPPSVSAAPGQKVTISCSGSSSDIGNNFVSWYQQLPGTAPKRLIYDNSKRPSGIPERFSGSKSGTSATLGITGLQTGDEADYYCGAWDTSLSAYVFGTGTKVTVL

SEQ ID NO: 109 - protein, 121 aa, synthetic construct
QVQLQQWGPGLVKPSETLSLTCAVYGGSFSGYYWSWIRQPPGKGLEWIGEINHSGSTNYNPSLKSRVTISVDTSKNQFSLKLSSVTAADTAVYYCARYSNYRHYYYGMDVWGQGTLVTVSS

SEQ ID NO: 110 - protein, 110 aa, synthetic construct
QSVLTQPPSVSAAPGQKVTISCSGSTSNVGNYYVAWYQKLPGEAPKVLIHDNNRRPSGIPVRFSGSKSGTSATLGITGLQTGDEAEYYCAVWDSSLNGYVFGGGTKLTVL

SEQ ID NO: 111 - protein, 116 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSLTAADTAVYYCARVNGGESDYWGQGTLVTVSS

SEQ ID NO: 112 - protein, 112 aa, synthetic construct
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGFWVFGGGTKLTVL

SEQ ID NO: 113 - protein, 118 aa, synthetic construct
QVQLQESGPGLVKPSGTLSLTCAVSGGSISSSNWWSWVRQPPGKGLEWIGEISHSGSTNYNPSLKSRVTISVDNSKNQFSLQLSSVTAADTAVYYCVSNASGRRGFAWGQGTLVTVSS

SEQ ID NO: 114 - protein, 116 aa, synthetic construct
QVQLQESGPGLVELSGTLSLTCAVSGVSISSRNWWSWVRQTPGKGLEWIGEINHSGSTNYNPSLKSRVTISVDTSKNQFSLKLSSVTAADTAVYYCARGGSDMDVWGKGTMVTVSS

SEQ ID NO: 115 - protein, 112 aa, synthetic construct
QSVLTQPPSVSGAPGQRITISCTGSSSNIGAGYDVQWYQQVPGKAPKHLIYGNSNRPSGVPDRFSGSKSGTSASLAITGLQAEDEADYFCQSYDSSLSGYVVFGGGTKLTVL

SEQ ID NO: 116 - protein, 117 aa, synthetic construct
QVQLQESGPGLVKPSETLSLTCAVSGGSISSSNWWSWVRQPPGKGLEWIGEISHTGSPNYNPSLASRVTISMDKSKNQFSLNLRSVTAADTSVYYCARYGRGAFDIWGQGTMVTVSS

SEQ ID NO: 117 - protein, 112 aa, synthetic construct
QAVLTQPSSVSGAPGQRVTISCTGSSSNIGAGYDVQWYQQLPGTAPKLLIYADSNRPSGVPDRFSGSKSGTSASLAITGLQAEDEADYYCQSYDSSLSGSKVFGTGTKLTVL

SEQ ID NO: 118 - protein, 117 aa, synthetic construct
QVQLVESGPGLVKPSGTLSLTCAVSGGSISSSDWWSWVRQPPGKGLEWIGEMRLSGTTNYNPSLKSRVAISMDKSKNHFSLNLSSVTAADTAVYYCANADYYTHHYWGQGTLVTVSS

SEQ ID NO: 119 - protein, 112 aa, synthetic construct
QAVLTQPSSVSGAPGQRVTISCTGSSSNIGAGYDVHWYQQLPGRAPKLLIYGNNQRPSGVPDRFSGSTSGTSASLAITGPQAEDEADYYCQSYDSSLNGIWVFGGGTKLTVL

SEQ ID NO: 120 - protein, 112 aa, synthetic construct
QVQLQESGPGLVKPSGTLSLTCAVSGVSISTTNWWSWVRQPPGKGLEWIGEINPSGSTNYNPSLKSRVTMSLDTSKNQFSLKLRSVTAADTALYYCATRDYWGQGTLVTVSS

SEQ ID NO: 121 - protein, 112 aa, synthetic construct
QAVLTQPSSVSGAPGQRVTISCTGSRSNIGANYDVHWYQQLPGTAPKLLIFNDNIRPSGVPDRFSGSKSGTSASLAITGLQAEDEADYYCQSYDSSLSGSVVFGGGTKLTVL

SEQ ID NO: 122 - protein, 117 aa, synthetic construct
QVQLQESGPGLVKPSGTLSLTCAVSGVSISTTNWWSWVRQPPGKGLEWIGEISHSGITNYNPSLQSRVTIALDKSKNHFSLNVNSVTAADTAVYYCASYWLGNFDYWGQGTLVTVSS

SEQ ID NO: 123 - protein, 112 aa, synthetic construct
QSVLTQPPSVSGAPGQRVTISCTGSSSNIGAGYEVHWYQQLPGRAPRLLIFDNNNRPSGVPDRFSASKSGTSASLAITGLRAEDEGDYYCQSYDSKRTPPYVFGTGTRVTVL

SEQ ID NO: 124 - protein, 118 aa, synthetic construct
QVQLQQSGAEVKKPGASVKVSCKASGYSFSKYGMSWVRQAPGQGLEWMGWINAGNGDTKYSQKFQGRVTITRDTSASTAYMELSSLKYEDTAIYYCARRLSYYGMDVWGQGTTVTVSS

SEQ ID NO: 125 - protein, 110 aa, synthetic construct
QAVLTQPSSVSAAPGQKVTISCSGSDSNIGNYYVWWYQQLPGAAPKLLIYDNHRRPSGVPDRFSGSKSGTSATLDITGLQTGDEADYYCGTWDGSLTGYVFGPGTKVTVL

SEQ ID NO: 126 - protein, 119 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCARGRVRGRWLPYYWGQGTLVTVSS

SEQ ID NO: 127 - protein, 117 aa, synthetic construct
QVQLQESGPGLVKPSETLSLTCAVSGVSISSHNWWSWVRQTPGKGLEWIGEISHSGITNYNPSLKSRVTISVDTSKNQLSLKLSSVTAADTAVYYCARGSTGAFDVWGQGTMVTVSS

SEQ ID NO: 128 - protein, 110 aa, synthetic construct; identical to SEQ ID NO: 110

SEQ ID NO: 129 - protein, 112 aa, synthetic construct
QAVLTQPSSVSGAPGQRVTISCTGSSSNIGAGYDVHWYQQLPGTAPKLLIYSNTNRPSGVPDRFSGSKSGTSASLAITGLQAEDEADYYCQSYDSSLSGLSVFGTGTKVTVL

SEQ ID NO: 130 - protein, 116 aa, synthetic construct
QLQLQESGPGLVEPSGTLSLTCAVSGVSISSRNWWSWVRQTPGKGLEWIGEISHSGIPNYNPSLASRVTISIDRSQNQFSLKLTSVTAADTAVYYCARGGGRFDFWGQGTLVTVSS

SEQ ID NO: 131 - protein, 112 aa, synthetic construct
QSVVTQPPSVSGAPGQRVTISCTGSSSNIGANYDVHWYQQLPGTAPKLLIYGNSNRPSGVPDRFSGSKSGTSASLAITGLQAEDEADYYCQSYDSSLSGSSVFGGGTKLTVL

SEQ ID NO: 132 - protein, 117 aa, synthetic construct
QVQLQQSGPGLVKPSGTLSLTCDVSGGSISSRNWWTWVRQPPGKGLEWIGEIYHSGSTNYNPSLESRVAMSVDKSRYQFSLRLSSVTAADTAVYYCARRRDGYFDYWGQGTLVTVSS

SEQ ID NO: 133 - protein, 118 aa, synthetic construct
QVQLQQSGPGLVKPFGRPCPLTCDVSGGSISSRNWWTWVRQPPGKGLEWIGEIYHSGSTNYNPSLESRVAMSVDKSRYQFSLRLSSVTAADTAVYYCARRRDGYFDYWGQGTLVTVSS

SEQ ID NO: 134 - protein, 112 aa, synthetic construct
QAVLTQPSSVSGAPGQRVTISCTGSSSNIGAGYDVHWYQQLPGTAPKLLIYGNRNRPSGVPDRFSGSKSGTSASLAITGLQAEDEADYYCQSYDSSLSGSKVFGTGTKVTVL

SEQ ID NO: 135 - protein, 110 aa, synthetic construct
VLTQPPSVSGAPGQRVTISCTGGSTNIGAGYDVHWYQQLPGTAPKLLIYGNNNRPSGVPDRFSGSQSGASASLAITGLQADDEADYYCQSYDSRLDGSKVFGTGTKVTVL

SEQ ID NO: 136 - protein, 116 aa, synthetic construct
QVQLQESGPGLVKPSETLSLTCAVSGVSISSHNWWSWVRQTPGKGLEWIGEMSHSGIPNYNPSLESRVTISLDKSKNQFSLILRSVTAADTAMYYCVGGSGSYSYWGQGTLVTVSS

SEQ ID NO: 137 - protein, 112 aa, synthetic construct
QAVLTQPSSVSGAPGQRVTISCTGTNSNIGAGYDVHWYQQFPGAAPKVLIYGNTNRPSGVPGRFSASKSGTSASLAITGLQAEDEADYYCQTYDSSLSGTWVFGGGTKLTVL

SEQ ID NO: 138 - protein, 117 aa, synthetic construct
QVQLQQWGPGLVKPSGTLSLTCAVSGVSISSSNWWSWVRQTPGKGLEWIGEISHSGSPNYNPSLKSRVTISVDTSKNQFSLKLTSVTAADSAVYYCARGGGPIPHIWGQGTTVTVSS

SEQ ID NO: 139 - protein, 112 aa, synthetic construct
QAVLTQPSSVSGAPGQRVTISCTGSSTNIGADFDVHWYQQLPGTAPKLLIHGDNNRPSGVPDRFSGSKSGTSAYLAISGLQAEDEADYYCQTYDSRSSGSRVFGGGTKVTVL

SEQ ID NO: 140 - protein, 117 aa, synthetic construct; identical to SEQ ID NO: 138

SEQ ID NO: 141 - protein, 110 aa, synthetic construct
QSVLTQPPSVSAAPGQKVTISCSGSSSNIGNYYVSWYQQLPGTAPKLLIYDNNKRPSGIPDRFSGSKSGTSATLGVTGLQTGDEADYYCGTWDSSLSAYVFGTGTKVTVQ

SEQ ID NO: 142 - protein, 118 aa, synthetic construct
QVQLQESGPGLVKSSETLSLICAVSGGSISSNNWWSWVRQPPGKGLEWIGEIHHSGTTINYNPSLKSRVTISVDKSKNQFSLQLNSVTPEDTAVYFCARSASGAFDIWGQGTMVTVSS

SEQ ID NO: 143 - protein, 112 aa, synthetic construct
QAVLTQPSSVSGAPGQRVTISCTGSSSNIGAGYDVQWYQQLPGTAPKLLIYADNNRPSGVPDRFSGSKSGTSASLAITGLQAEDEADYYCQSYDSSLSGSKVFGTGTKVTVP

SEQ ID NO: 144 - protein, 120 aa, synthetic construct
QVQLQESGPGLVELSGTLSLTCAVSGVSISSRNWWSWVRQTPGKGLEWIGEISHSGIPNYNPSFVSRVTISIDRSQNQFSLKLTSMTAADTAVYYCARLYSGYGHGMDVWGQGTTVTVSS

SEQ ID NO: 145 - protein, 112 aa, synthetic construct
QAVLTQPSSVSGAPGQRVTISCTGSSSNIGAGYDVHWYQQLPGTAPKLLIYGNSNRPSGVPDRFSGSKSGTSASLAITGLQAEDEADYYCQSYDSSLSGLYVFGTGTKVTVL

SEQ ID NO: 146 - protein, 115 aa, synthetic construct
QVQLQESGPGLVKPSETLSLTCAVSGVSISTTNWWSWVRQPPGKGLEWIGEISHSGITNYNPSLKSRVTISVDKAKNQFSLMLNSVTAADTAVYYCARGYGMDVWGQGTMVTVSS

SEQ ID NO: 147 - protein, 117 aa, synthetic construct
QVQLQQWGPGLVKPSGTLSLTCAVSGGSISSSNWWSWVRQFPGKGLEWIGEMSHTGSTNYNPSFKSRVTISVDKSKNQFSLKLSPVTAADTAVYYCARGSTGAFDVWGQGTMVTVSS

SEQ ID NO: 148 - protein, 112 aa, synthetic construct
QSVLTQPPSVSGAPGQRVTISCTGSSSNIGAGYDVHWYQQLPGTAPKVLIYGNSNRPSGVPDRFSGSKSGTSASLAITGLQAEDEADYYCQSYDSSLSGSVVFGGGTKLTVL

SEQ ID NO: 149 - protein, 111 aa, synthetic construct
QAVLTQPSSVSGAPGQRVTISCTGSSSNIGAGYDVNWHQQFPGTAPKVLIFGDNARPSGVPDRFYASKSGTSASLTIIGVQSDDEADYYCSTWDDSLNAVVFGGGTTLTVL

SEQ ID NO: 150 - protein, 116 aa, synthetic construct
VQLQESGPGLVKPSGTLSLTCAVSGGSISSGNWWSWVRQPPGRGLEWIGEISHSGTINYNPSLKSRVTISVDTSKNQFSLKLSSVTAADTAVYYCARRADGYFQPWGQGTLVTVSS

SEQ ID NO: 151 - protein, 110 aa, synthetic construct
VLTQPPSVSGAPGQRVTISCTGSDSNIGAGYDVHWYQQYPGIAPKLLIYAHHKRPSGVPDRFSGSTSGTSASLAITGLQAEDEADYYCQSYDSSLSGHYVFGTGTQVSVL

SEQ ID NO: 152 - protein, 117 aa, synthetic construct
QVQLQESGPGLVKPSETLSLTCAVSGGSISSNNWWSWVRQTPGKGLEWIGEIYHSGNTNYNPSLKSRVTISVDKSKNQFSLKLNSVTAADTAVYYCARGRQGAFDPWGQGTLVTVSS

SEQ ID NO: 153 - protein, 112 aa, synthetic construct
QAVLTQPSSVSGAPGQRVTISCTGSSSNIGAGYDVQWYQQLPGTAPKLLIYADSNRPSGVPDRFSGSKSGTSASLAITGLQAEDEADYYCQSYDSSLSGSKVFGTGTKVTVL

SEQ ID NO: 154 - protein, 116 aa, synthetic construct
QVQLQESGPGLVKPSGTLSLTCDVSGGSISSNNWWSWVRQSPGKGLEWIGEIIHTGRTNYNPSLTSRVTILIDKSKNQFSLKLTSVTPEDTALYYCARLRGPFDIWGQGTMVTVSS

SEQ ID NO: 155 - protein, 112 aa, synthetic construct
QAVLTQPSSVSGAPGQRVIISCTGSSSNIGAGYDVHWYQQLPGTAPKLLIYGNTNRPSGVPDRFSGSKSGTSASLAITGLQAEDEADYYCQSYDSSLSGNYVFGPGTKVTVL

SEQ ID NO: 156 - protein, 112 aa, synthetic construct
QAVLTQPSSVSGAPGQRVAISCTGSSSNIAAGYDVQWYQQLPGTAPKLLIYGNSNRPSGVPDRFSGSKSGTSASLAITGLQAEDEADYYCQSYDSSLSVLYVFGTGTKVTVL

SEQ ID NO: 157 - protein, 122 aa, synthetic construct
QLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAKRPRNSGYLGAFDIWGQGTMVTVSS

SEQ ID NO: 158 - protein, 111 aa, synthetic construct
DVVMTQSPLSLAVTLGQPASISCRSSQSLLHSSGYKFLNWYLQKPGQSPQLLIYLGSNRASGVPDRFSGSGSGTDFTLKISRVEAEDVGVYYCMQALQSPTFGGGTKVEIK

SEQ ID NO: 159 - protein, 118 aa, synthetic construct
EVQLVESGGGLVQPGGSLRLSCTASGFTFSSFSMNWVRQAPGKGPEWVSAISGSGGGTYYADSVKGRFTISRDNAKNSLYLQMSSLRAEDTAVYYCARVRVPQAFDIWGQGTMVTVSS

SEQ ID NO: 160 - protein, 107 aa, synthetic construct
DIQLTQSPSSLSASVGDRVTITCRASQNINNFLNWYQQKPGNVPKLLIYAASSLQSGVPSRFSGSGSGTDFTLTISSLQPEDFATYYCQQSYSPLSTFGQGTKVEIK

SEQ ID NO: 161 - protein, 120 aa, synthetic construct
QVQLVQSGAEVKKPGSSVKVSCKASGYTFTGYYMHWVRQAPGQGLEWMGRINPNSGGTNYAQKFQGRVTMTRDTSISTAYMELSRLRSDDTAVYYCARSVSVVTDAFDIWGQGTTVTVSS

SEQ ID NO: 162 - skipped

SEQ ID NO: 163 - protein, 113 aa, synthetic construct
QLQLQESGPGLVKPSQTLSLTCTVSGASISSSNWWSWVRQTPGKGLEWIGEISHSGSPNYNPSLKSRVTISVDTSKNQFSLKVSSVTAADTAVYYCAREPRYWGQGTLVTVSS

SEQ ID NO: 164 - protein, 112 aa, synthetic construct
QAVLTQPSSLSGAPGQRVTISCTGSSSNIGAGYDVHWYQQLPGTAPKLLISGNRNRPSGVPDRFSGSKSGTSASLAITGLQAEDEADYYCQSYDSSLSGSFVFGTGTKVTVL

SEQ ID NO: 165 - protein, 117 aa, synthetic construct
QLQLQESGPGLVEPSGTLSLTCAVSGVSISTRNWWSWVRQTPGKGLEWIGEISHSGIPNYNPSFVSRVTISVDASKNQLSLKLTSVTVADTAVYYCSRKGVDAFDIWGQGTMVTVSS

SEQ ID NO: 166 - protein, 116 aa, synthetic construct
QLQLQESGPGLVEPSGTLSLTCAVSGVSISTRNWWSWVRQTPGKGLEWIGEISHSGIPNYNPSFVSRVTISVDASKNQLSLKLTSVTVADTAVYYCSRKGVDAFDIWGQGTMVTVS

SEQ ID NO: 167 - protein, 111 aa, synthetic construct
QSVVTQPPSVSAAPGQKVTISCSGSSSNIGNYYVAWYQQVPGAAPKLLIYDTNKRPSGIPDRFSGSKSGTSATLDITGLRTGDEADYYCGTWDSSLDTDVVFGGGTKLTVL

SEQ ID NO: 168 - protein, 118 aa, synthetic construct
QVQLQESGPGLVKPSETLSLTCAVSGGSISSGSWWSWVRQAPGKGLEWIGEISHSGTTTYNPSLKSRVTISLDKSTSHLSLSLKSVTAADTAVYYCARELGGGAYDIWGQGTIVTVSS

SEQ ID NO: 169 - protein, 110 aa, synthetic construct
QSVLTQPPSVSAAPGQKVTISCSGSSSNIGNYHVSWYQQLPGTAPKLLIYDNNKRPSGIPDRFSGSKSGTSATLDITGLQTGDEADYYCGTWDSSLSAVLFGRGTKLTVL

SEQ ID NO: 170 - protein, 115 aa, synthetic construct
QLQLQESGPGLVKPSGTLSLTCAVSGGSISSGYYWSWIRQPPGKGLEWIGEINHSGSTNYNPSLKSRVTISVDTSKNQFSLKLSSVTAADTAVYYCARLRGSGRWGQGTLVTVSS

SEQ ID NO: 171 - protein, 112 aa, synthetic construct
QAVLTQPSSVSGAPGQRLTISCTGSTSNIGAGYDVQWYQKLPGAAPKLLVYGNTNRPSGVPDRFSGSKSGTSASLAITGLQAEDEGHYYCQSYDSSLSGWVVFGGGTKLTVL

SEQ ID NO: 172 - protein, 114 aa, synthetic construct
QLVQSGPGLVKPSGILSLTCAVSGGSITSSNWWSWVRQPPGKGLEWIGEISHSGSTNYNPSLKSRVTMSVDKSKNQLSLKLSSLTAADTAVYYCARGGSSLPIWGQGTTVTVSS

SEQ ID NO: 173 - protein, 111 aa, synthetic construct
QAVLTQPSSVSGAPGQTVTISCTGSSSNIGADYDVHWYQQLPGTAPKLLIYGNNNRPSGVPDRFSGSKSGASASLAITGLQADDEADYYCHSYDSTRSGLYIFGTGTRVIV

SEQ ID NO: 174 - protein, 114 aa, synthetic construct
QLQLQESGPGLVKPSGTLSLTCAVSGVSISTTNWWSWVRQPPGKGLEWIGEISHSGITNYNPSLQSRVTISVDKSKNQFSLKLSSVTAEDTAVYYCTTGSSGYWGQGTLVTVSS

SEQ ID NO: 175 - protein, 112 aa, synthetic construct
QSVLTQPPSVSGAPGQRVTISCTGSSSNIGADYDVQWYQQLPGTAPKLLIYANNNRPSGVPDRFSGSKSGTSASLAITGLQAEDEADYYCQSYDSSLSGLRVFGGGTKLTVL

SEQ ID NO: 176 - protein, 118 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGRRSSWIDYWGQGTLVTVSS

SEQ ID NO: 177 - protein, 111 aa, synthetic construct; identical to SEQ ID NO: 99

SEQ ID NO: 178 - protein, 117 aa, synthetic construct
QVQLQESGPGLVKPSGTLSLTCAVSGVSISTTNWWSWVRQPPGKGLEWIGEISHSGITNYNPSLQSRVTISVDTSKNQFSLKLNSVTAADTAVYYCARVGEGAFKDLGQGTLVTVSS

SEQ ID NO: 179 - protein, 113 aa, synthetic construct
QLQLQESGPGLVKPSGTLSLNCAVSGVSISSTNWWSWVRQFPGKGLEWIGEINHSGTTNYNPSLKSRVTISVDTSKNQFSLQLNSVTPEDTAVYFCAQHLTVWGQGTLVTVSS

(19) United States
(12) Patent Application Publication
(10) Pub. No.: US 2026/0347441 A1
(43) Pub. Date: Nov. 5, 2026

(54) ANTI-MONOMETHYL AURISTATIN ANTIBODIES AND ANTIBODY FRAGMENTS

(71) Applicant: The Research Foundation for The State University of New York, Amherst, NY (US)

(72) Inventors: Joseph P. BALTHASAR, Lancaster, NY (US); Brandon M. BORDEAU, West Seneca, NY (US); Toan Duc NGUYEN, Williamsville, NY (US); Joseph Ryan POLLI, Cambridge, MA (US)

(21) Appl. No.: 19/105,702

(22) PCT Filed: Aug. 24, 2023
(86) PCT No.: PCT/US2023/072852

§ 371 Date: Feb. 21, 2025

Related U.S. Application Data
(60) Provisional application No. 63/373,367, filed on Aug. 24, 2022.
(60) Provisional application No. 63/520,689, filed on Aug. 21, 2023.

(51) Int. Cl. C07K 16/18; A61K 39/395; A61P 39/02

(52) U.S. Cl. CPC C07K 16/18; A61K 39/3955; A61P 39/02; C07K 2317/622

(57) ABSTRACT

Provided are compositions and methods for reducing off-target toxicity of antibody-drug conjugates (ADCs). The compositions comprise an ADC, and an agent targeted to the drug (payload) that is delivered by or derived from the ADC. The ADC and the agent targeted to the payload may be delivered together or separately in the treatment of various conditions (such as tumors) by ADCs. Examples of agents targeted to payload include antibodies, fragments, or modifications thereof.

### CROSS-REFERENCE TO RELATED APPLICATIONS

This application claims priority to U.S. provisional patent application No. 63/373,367, filed Aug. 24, 2022, and to U.S. provisional application No. 63/520,689, filed Aug. 21, 2023, the entire disclosures of each of which are incorporated herein by reference.

### SEQUENCE LISTING

The instant application contains a Sequence Listing which has been submitted in .xml format and is hereby incorporated by reference in its entirety. Said .xml copy was created on Aug. 24, 2023, is named “011520_01784_Balthasar_PCT.xml” and is 183,732 bytes in size.

### BACKGROUND OF THE DISCLOSURE

Anti-cancer antibody-drug conjugates (ADCs) are being employed for targeted delivery of drugs, which may be toxins or other cell growth inhibitors (termed here as drug or payload molecules) to cancer cells. Twelve ADCs are currently marketed in the US, and approximately 100 ADCs are in development (Chau et al., Lancet. 2019; 394(10200):793-804; Coats et al., Clin Cancer Res. 2019. Epub 2019/04/14. doi: 10.1158/1078-0432.CCR-19-0272; Wolska-Washer et al., Drug Saf. 2019; 42(2):295-314; Beck et al., Nat Rev Drug Discov. 2017; 16(5):315-37). However, clinical application of ADCs has been somewhat disappointing; many ADCs have failed in clinical trials as a result of substantial off-target toxicity, which has limited tolerable doses below levels needed for tumor eradication (Coats et al., Clin Cancer Res. 2019. Epub 2019/04/14. doi: 10.1158/1078-0432.CCR-19-0272; Kim et al., Biomol Ther (Seoul). 2015; 23(6):493-509; de Goeij et al., Curr Opin Immunol. 2016; 40:14-23; Khera et al., BioDrugs. 2018; 32(5):465-80). As a result of associated toxicity to non-target sites, the early promise of ADCs is not fully realized and therefore, there is an ongoing need in the area of cancer therapy to develop new approaches to minimize non-target toxicity of therapeutic payload drug molecules without compromising their anti-tumor efficacy.

### SUMMARY OF THE DISCLOSURE

This disclosure provides compositions and methods for reducing off-target toxicity of ADCs. For example, the present compositions and methods can be used for treatment of tumors with ADCs, while reducing off-target toxicity of the ADCs. The drug in the ADC may be referred to herein as “payload”. The compositions comprise an ADC and an agent targeted to the payload that is delivered by or derived from the ADC. The agent targeted to the payload is termed herein as “payload-binding agent” or PBA. The ADC and the payload-binding agent may be provided in the same composition or in different compositions. The payload binding agent may be a peptide or an antibody or a fragment or an antibody mimetic or a modification thereof directed to the ADC payload, and which binds to the payload. If the payload binding agent is an antibody or a fragment or modification thereof, it may be referred to as “anti-payload antibody”.

In an aspect, this disclosure provides a method for inhibiting or preventing the growth of one or more tumors comprising administering to an individual in need of treatment, an ADC and a payload binding agent, wherein the payload binding agent has specific affinity for the ADC payload. The ADC and the payload binding agent may be administered in the same composition or different compositions, via the same routes or via different routes, or using the same regimen or different regimen.

In an aspect, this disclosure provides peptides or antibodies or antibody fragments or modifications, which are specific for an ADC payload molecule. The anti-payload antibody may be whole immunoglobulin molecules such as polyclonal or monoclonal antibodies or chimeric antibodies including humanized antibodies. The antibody fragments or modification can be antigen-binding fragments thereof, including, but not limited to, Fab, F(ab′), F(ab′)2, Fv, dAb, Fd, CDR fragments, single-chain antibodies (scFv), bivalent single-chain antibodies, single-chain phage antibodies, diabodies, or single domain antibodies (nanobodies) and the like. Antibody mimetics may include affibodies, nanofitins, or the like. The fragments of the antibodies may be produced synthetically or by enzymatic or chemical cleavage of intact immunoglobulins or may be genetically engineered by recombinant DNA techniques. These techniques are well known in the art. The antibody or fragments or modifications thereof may be modified so as to impart longer half-life, stability and the like. In an embodiment, the disclosure provides antibodies or fragments or derivatives thereof directed to several ADC payload, including full length antibodies, scFv, Fab and other fragments directed specifically to auristatins (e.g., monomethyl auristatin E [MMAE], monomethyl auristatin F [MMAF], monomethyl auristatin F methyl ester, monomethyl auristatin D, and the like.) In non-limiting embodiments, the anti-payload specifically binds to MMAE and/or MMAF.

### BRIEF DESCRIPTION OF THE FIGURES

FIG. 1: Provided are the top 5 scFv clones that were identified following biopanning and screening of the immunized murine scFv phage library. The fraction bound represents the ELISA signal for phage bound to MMAF-biotin-streptavidin immobilized on an ELISA plate, for each clone with or without MMAE or T-vc-MMAE spiked in. Bars represent the mean of duplicate samples with standard deviation error bars. Clones 1B3, 1H2, 3B8, 2E8 and 2C2 had a >50% decrease in the binding fraction with the addition of 30-100 nM free MMAE, with no change in binding with the addition of 100 nM T-vc-MMAE.

FIG. 2: Provided are the sequences from the top 5 clones identified from the 1^{st} panning. Complementarity determining regions identified using the North definition are shown in boxed regions. Clone 1H2 has an identical sequence to clones 2E8 and 2C2 from FIG. 1. The sequence in the top line is SEQ ID NO:31.

FIG. 3: A random mutagenesis scFv library was built from clones 1B3, 1H2 and 3B8 (FIGS. 1 and 2) to identify clones with increased MMAE binding affinity. Shown is the fractional binding signal for four 96-well plates from two different panning dissociation steps (3-hour and 24-hour). The fractional binding signal for the 3-hour plates is the signal observed with 10 nM free MMAE spiked in divided by a control well with no MMAE. The fractional signal for the 24-hour plates is with 1 nM free MMAE spiked in.

FIG. 4: Clones from the screening results in FIG. 3 were sequenced. The parent scFv sequences that were used to build the mutagenesis library are provided (1B3, 1H2, 3B8) with 1H2 set as the reference sequence. Complementarity determining regions identified using the North definition are shown in boxed regions. The amino acid sequence for the variable domains of clone MA24E2 were used to inform the development of the humanized variant ABC3315. The sequence of the 1B3 construct in the top line is SEQ ID NO:31.

FIG. 5: The binding of ABC3315 to MMAF-biotin-streptavidin was decreased with increasing concentrations of free MMAE and MMAF (IC50: ~1 nM), whereas trastuzumab-vc-MMAE (T-vc-MMAE) did not compete for binding. Points represent the mean of samples in triplicate with standard deviation error bars.

FIG. 6: Shown are kinetic titration binding SPR sensorgrams for ABC3315 to MMAF-peg11-biotin neutravidin (left), MMAE to ABC3315-peg12-biotin-streptavidin (middle) and polatuzumab vedotin (PV) to ABC3315-peg12-biotin-streptavidin (right). Best fit values for the association rate constant (Kon) dissociation rate constant (koff) and equilibrium dissociation rate constant (KD) are provided in the insets. No binding signal was observed for PV to ABC3315-peg12-biotin-streptavidin.

FIG. 7: ABC3315 selectively inhibits MMAE toxicity. (A) RAMOS cells were incubated with MMAE (10 pM-100 nM), with or without co-incubation with 500 nM ABC3315. ABC3315 increased the IC50 of free MMAE by 800-fold. (B) Polatuzumab vedotin (PV), a clinically-approved anti-CD79b ADC that incorporates MMAE as the payload molecule, was incubated with CD79b+ Ramos cells at concentrations of 3 pM-30 nM, with or without coincubation with 500 nM ABC3315. ABC3315 did not alter the on-target cellular cytotoxicity of PV (IC50: 0.12 nM alone vs 0.13 nM with ABC3315). (C) ABC3315 (500 nM) increased the IC50 of free MMAE against SKBR3 cells by greater than 500-fold. (D) HER2+ SKBR3 cells were incubated with T-vc-MMAE with and without anti-MMAE fab with negligible changes in cellular cytotoxicity (IC50: 0.03 nM alone vs 0.04 nM ABC3315). Points represent the mean of triplicate wells with standard deviation error bars.

FIG. 8: ABC3315 does not alter the efficacy of PV. Nu/J mice bearing Ramos xenografts at a volume of ~250 mm³ were randomized to receive PBS, 1 or 3 mg/kg PV with or without a 12-fold molar excess of ABC3315 (n=8/group). (A) The tumor volumes over time for each group are provided with standard deviation error bars. Mice were sacrificed at a tumor volume of 2000 mm³. (B) The probability of survival over time for each group is provided. Based on the Log-Rank test, the co-treatment of ABC3315 did not significantly alter the group survival for mice treated with PV at a dose of 1 mg/kg (p=0.075) or 3 mg/kg (p=0.89).

FIG. 9: ABC3315 decreases the body weight loss of mice treated with 120 mg/kg PV. Swiss Webster mice were intravenously injected with 120 mg/kg PV with PBS or a 3-fold molar excess of ABC3315 (n=5/group). (A) The body weight loss over time for each group is provided; points represent the mean percent change in body weight and error bars depict standard deviations. (B) The mean percentage weight change at nadir for each group is provided with standard deviation error bars. Mice administered ABC3315 with PV had a significantly decreased group nadir body weight loss from a mean of 11.9±7.0% for mice treated with PV+PBS to 4.1±2.1% for mice treated with PV+ABC3315 (p=0.045).

FIG. 10: Evaluation of ABC3315-WEDD HSA binding. Graphs showing ABC3315-WEDD injected over a SPR chip with immobilized human serum albumin at a range of concentrations. Top: The observed association and dissociation curves were fit to a 1:1 Langmuir binding model to obtain binding rate constants. (Bottom) The observed Rmax for each concentration was used to estimate an equilibrium dissociation rate constant. Fit values for binding parameters are provided in the insets of each panel.

FIG. 11: Evaluation of ABC3315-WE human serum albumin (HSA) binding. ABC3315-WE was injected over a SPR chip with immobilized human serum albumin at a range of concentrations. Top: The observed association and dissociation curves were fit to a 1:1 Langmuir binding model to obtain binding rate constants. (Bottom) The observed Rmax for each concentration was used to estimate an equilibrium dissociation rate constant. Fit values for binding parameters are provided in the insets of each panel.

FIG. 12: Affinity matured scFv sequences using ABC3315 as the reference sequence. The sequence of the top line is SEQ ID NO:31.

FIG. 13: ELISA comparison of ABC3315 Mutants. ABC3315 mutants were evaluated for retained binding activity using an indirect ELISA method. All mutants, except for VH_F37V, showed a similar binding signal for MMAF-peg11-biotin-neutravidin across the concentrations of Fab that were tested.

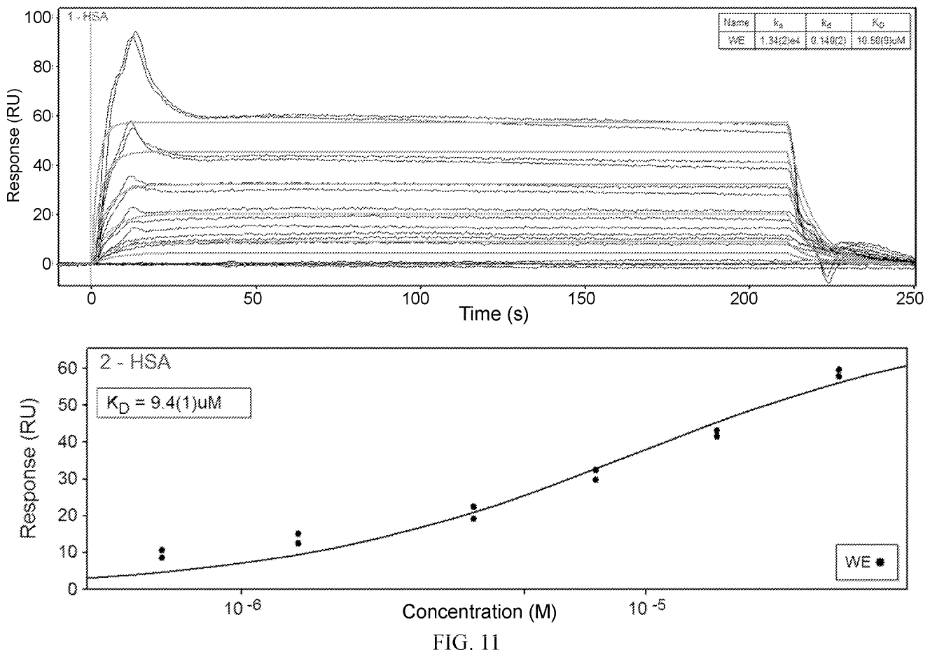

FIG. 14: Competitive Cell Cytotoxicity assay. The VH_F27L mutants' inhibition of MMAE mediated cellular cytotoxicity in comparison to ABC3315 was evaluated. Greater cell survival was observed across the range of MMAE concentrations with VH_F27L co-treatment relative to ABC3315 co-treatment.

FIG. 15: Graph showing the concentration of free MMAE in plasma and red blood cells following administration of 100 mg/kg TvcMMAE alone or in combination with ABC3315 was evaluated. ABC3315 decreased the free concentration of MMAE in plasma by 85% and red blood cells by 73%.

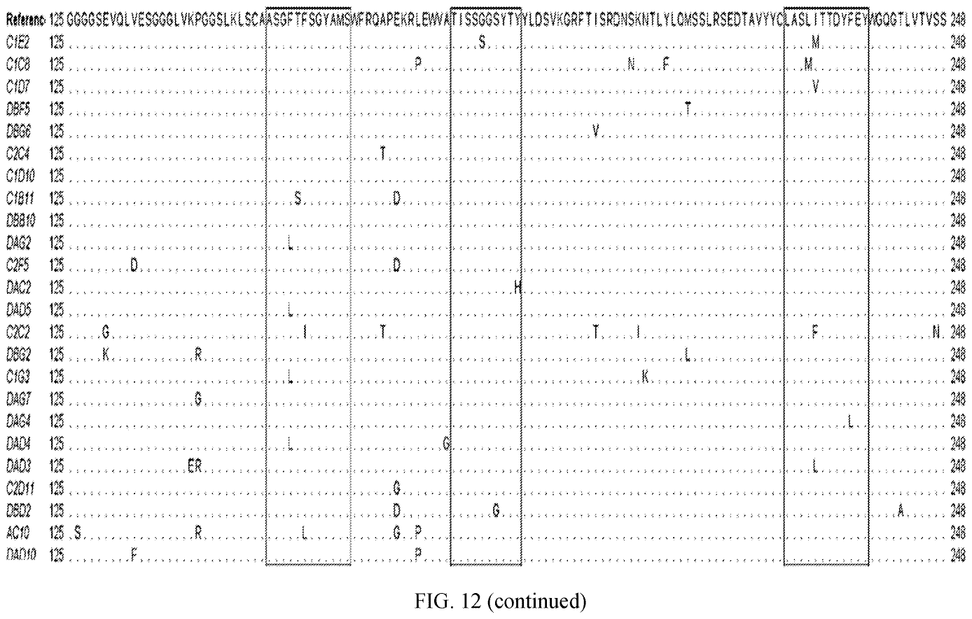

FIG. 16: Graphs showing results from TvcMMAE administration at a dose of 80 mg/kg alone or in combination with ABC3315. TvcMMAE administered alone resulted in a significant decrease in the white blood cell count and red blood cell count relative to mice administered a PBS vehicle. Mice that were administered TvcMMAE in combination with ABC3315 did not exhibit a significant decrease in white blood cells or red blood cells relative to control mice.

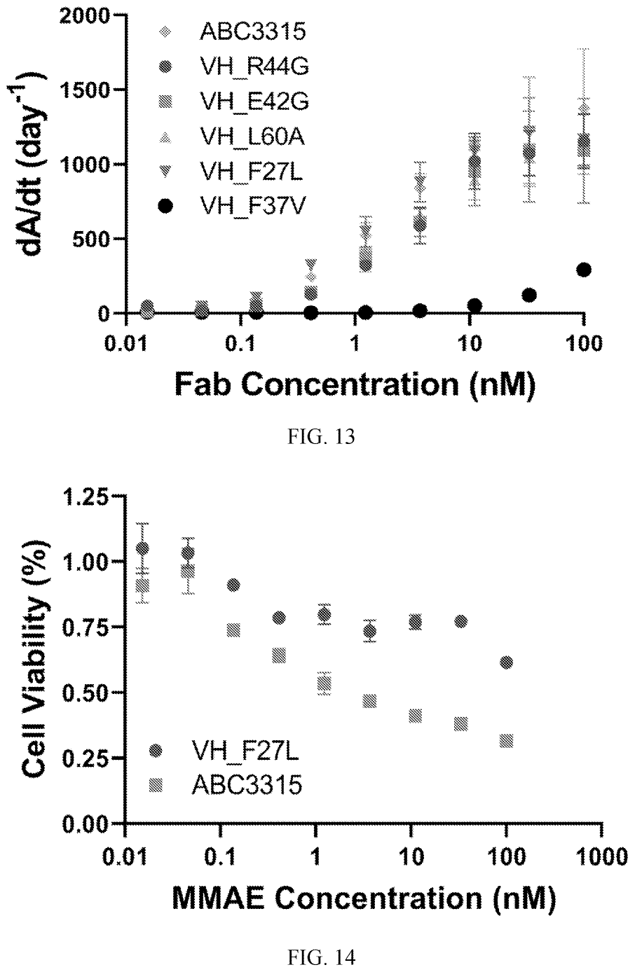

FIG. 17: Data showing the impact of ABC3315 on the efficacy of TvcMMAE in a HER2+/HER2− bystander xenograft model was evaluated. No significant difference was observed between mice administered 3 mg/kg TvcMMAE alone or in combination with ABC3315. A representative tumor is provided on the right.

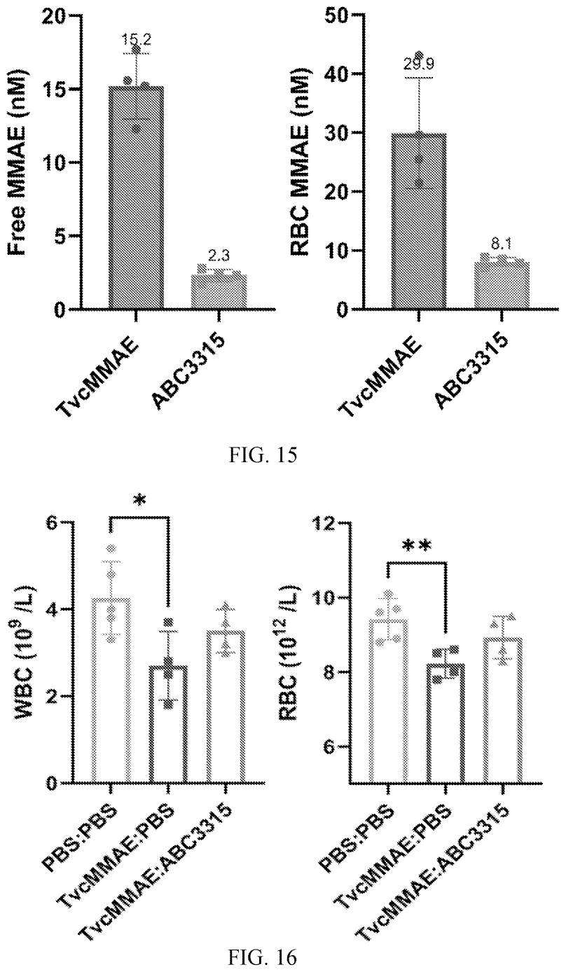

FIG. 18: Graph showing ABC3320 decreases MMAE ADC toxicity against differentiating neutrophils. Human peripheral blood mononuclear cells were incubated with trastuzumab-vc-MMAE (TvcMMAE) alone or in combination with ABC3320. Flow cytometry was used to evaluate the number of neutrophils following TvcMMAE treatment using CD66b+ as a neutrophil marker. ABC3320 increased the IC50 of TvcMMAE from 1.9 nM to 27 nM.

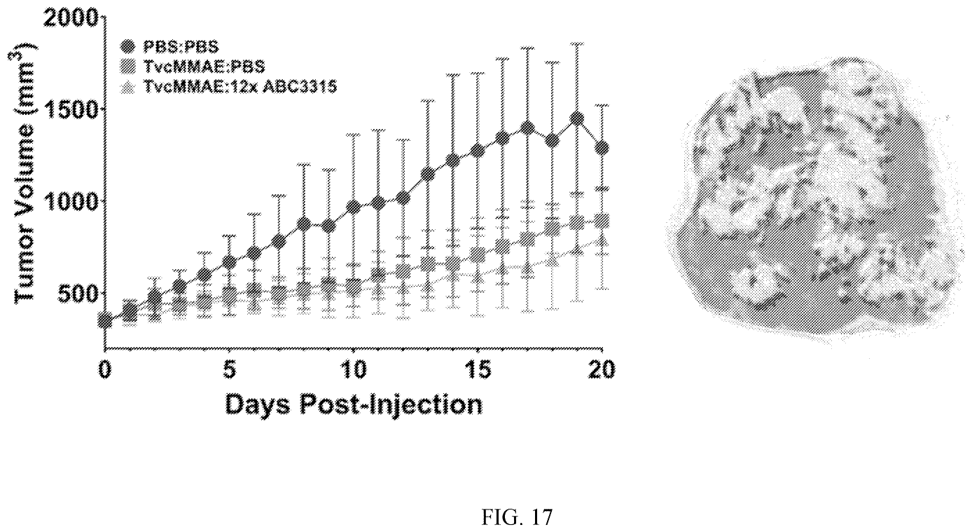

FIG. 19: Graph showing ABC3320 co-treatment decreased the body weight loss that was observed in rats treated with 25 mg/kg trastuzumab-vc-MMAE (ADC) relative to rats treated with TvcMMAE alone.

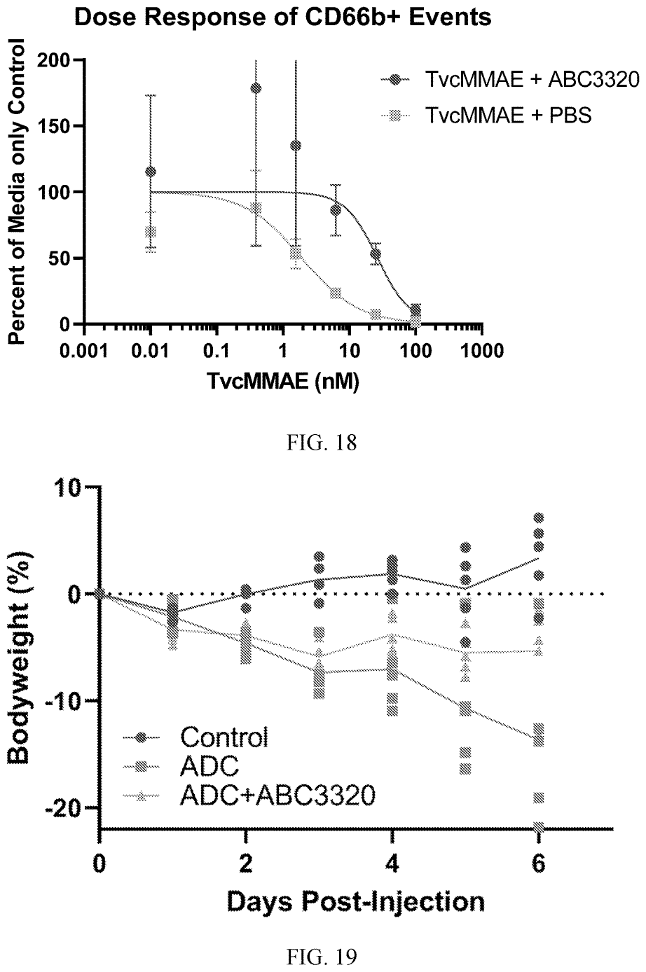

FIG. 20: Graphs showing ABC3320 decreases hematologic toxicity in rats following a 25 mg/kg dose of TvcMMAE.

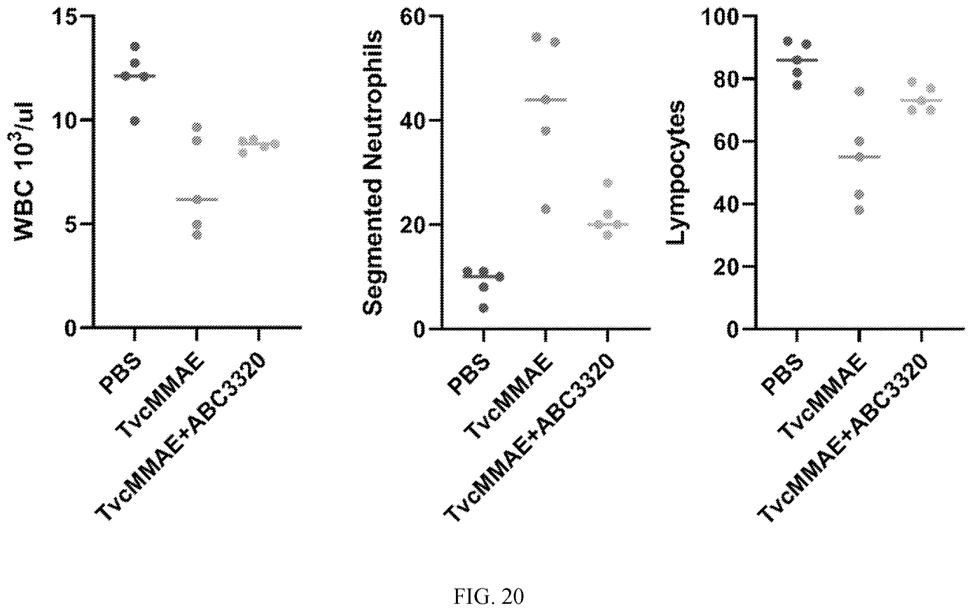

FIG. 21: Graphs showing ABC3320 decreases trastuzumab-vc-MMAE (25 mg/kg) mediated liver toxicity in rats.

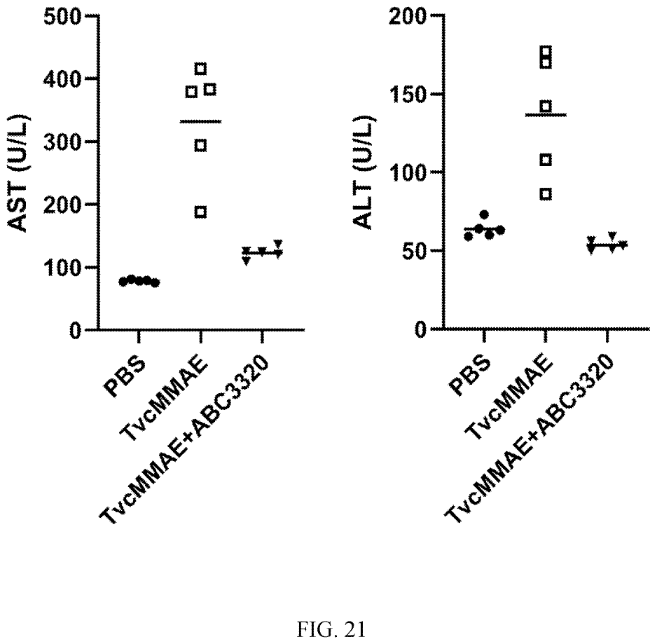

FIG. 22: Graphs showing ABC3320 does not decrease the efficacy of polatuzumab vedotin (PV) in mice bearing Ramos xenografts and does not decrease the efficacy of trastuzumab-vc-MMAE (TvcMMAE) in mice bearing a mixed xenograft model of HER2+ NCI-N87 cells and HER2− MCF7 cells.

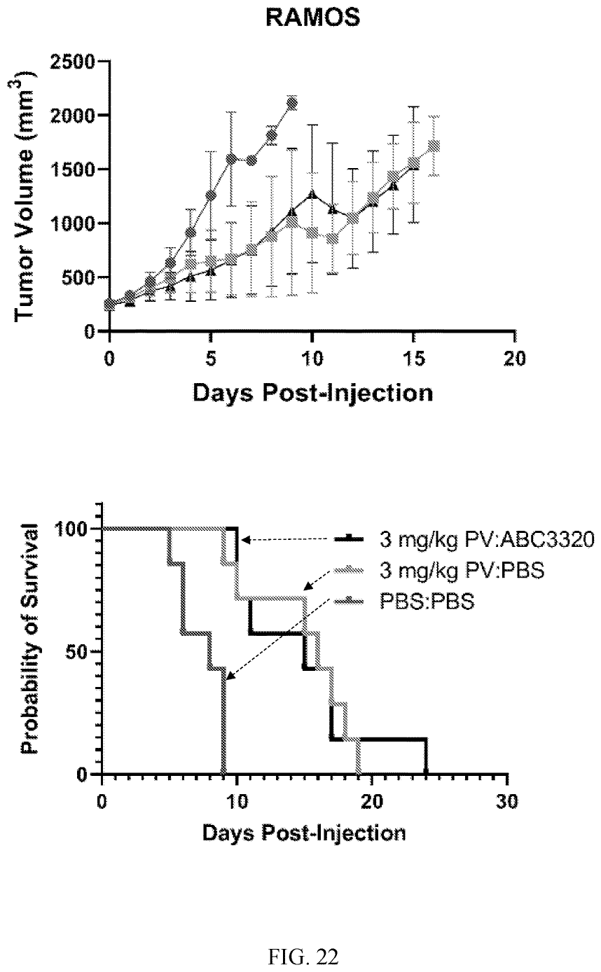

FIG. 23: Graph showing ABC3320 decreases free MMAE concentration in plasma following TvcMMAE administration. Swiss-Webster mice were intravenously injected with a 25 mg/kg dose of TvcMMAE administered with phosphate buffered saline (labeled as PBS) or with ABC3320 (98 mg/kg, with 65.3 mg/kg given in-combination with TvcMMAE at time=0 and 32.7 mg/kg given at 24 hours). Mice were sacrificed at a range of timepoints and whole blood was collected. Plasma and red blood cells were separated by centrifugation. Plasma samples (100 μl) were placed into wells of a rapid equilibrium dialysis device (Thermo Scientific™, 90006) and free MMAE was separated from bound MMAE (plasma protein bound or ABC3320 bound) following manufacturer recommendations. Free and bound MMAE concentrations were determined using LC-MS/MS. Free concentrations of MMAE were on average 200× lower for samples obtained from mice treated with ABC3320 when compared to samples obtained from mice administered TvcMMAE with PBS. Increased bound concentrations of MMAE in plasma were observed for mice administered ABC3320, which is consistent with pharmacokinetic expectations (where binding to ABC3320 is expected to decrease the apparent volume of distribution of released MMAE).

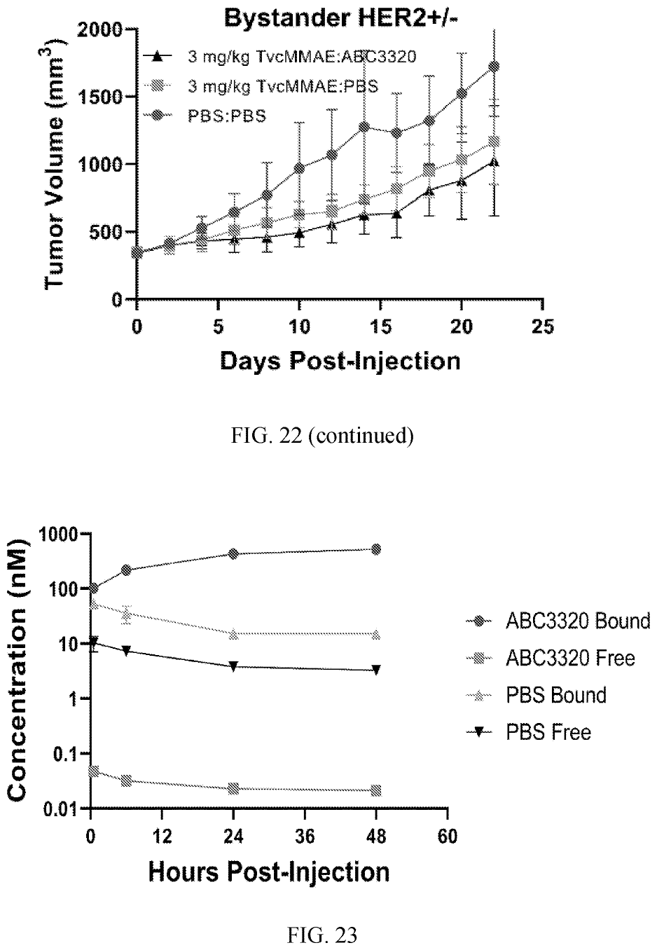

### DETAILED DESCRIPTION OF THE DISCLOSURE

The present disclosure provides compositions and methods for treatment of diseases (e.g., cancer) using ADC, while reducing off-target toxicity associated with the ADC. The method comprises administering to an individual in need of treatment an ADC and one or more payload binding agents directed to the drug portion of the ADC. The payload binding agent is effective in reducing the non-target toxicity of the ADC or the non-target toxicity derived from the free drug dissociated therefrom. The compositions comprise antibodies (including fragments or modifications thereof), which are directed to the drug of the ADC. Compositions are also provided that comprise the ADC and an agent (such as an antibody) which is directed to the drug that makes up the ADC. The agents bind to free drug and may or may not bind to the drug when it is part of the ADC. The present disclosure relates in part to PCT application PCT/US2020/063453, and its publication, published as PCT publication WO 2021/113740, published Jun. 10, 2021, the entire disclosure of which is incorporated herein by reference.

An ADC comprises an antibody group, a linking group, and a drug group. The antibody group targets an antigen, such as a tumor cell antigen, the linking group is used to attach the drug group to the antibody group, and the drug group is an agent that is cytotoxic to the targeted cell. The antibody group may be referred to as an antibody or by the name of the antibody. Similarly, the drug group may be referred to as a drug or by the name of the drug.

The term “treatment” as used herein refers to reduction or delay in one or more symptoms or features associated with the presence of the particular condition being treated. Treatment does not necessarily mean complete cure and does not preclude relapse, but may be used in connection with any such relapse.

The term “therapeutically effective amount” as used herein is the amount sufficient to achieve, in a single or multiple doses, the intended purpose of treatment. The exact amount desired or required will vary depending on the mode of administration, patient specifics and the like. Appropriate effective amounts can be determined by one of ordinary skill in the art (such as a clinician) with the benefit of the present disclosure.

Where a range of values is provided in this disclosure, it should be understood that each intervening value, to the tenth of the value of the lower limit of that range, and any other intervening value and ranges in that stated range are encompassed within the disclosure, unless clearly indicated otherwise. The upper and lower limits of these smaller ranges may independently be included in the smaller ranges encompassed within the disclosure.

As used in this disclosure, the singular forms include the plural forms and vice versa unless the context clearly indicates otherwise. The indefinite articles “a” and “an” as used in the specification and claims should be understood to mean “at least one” unless clearly indicated otherwise.

A general reference to an antibody in this disclosure is also intended to include all full-length antibodies, antibody fragments containing antigen binding domains, as well as modified antibodies or fragments containing substitutions or modifications of the amino acid residues, and including where antibody fragments, modified or not, may be linked together via covalent linkages with or without linkers. When a reference is made to an antibody being “targeted to payload” or “directed to payload” or similar phrases, it means the antibody has specific affinity for the payload, when the payload is in the form of free payload, including when it is cleaved from the ADC. The anti-payload antibody may or may not bind to the payload when the payload is conjugated to the antibody part of the ADC.

The terms “off-target” and “non-target” in reference to toxicity refer to toxicity that accompanies the administration of many chemotherapeutic agents. While the intended purpose of administration of a chemotherapeutic agent is to reduce or inhibit the growth of tumors or any accompanying metastases, the growth, function, and/or physiology of normal cells are often adversely affected in the course of treatment of cancer. A reduction of off-target or non-target toxicity is intended to reduce any adverse effects on the non-tumor or non-metastatic cells.

The term “payload binding agent” (PBA) as used in this disclosure refers to an agent that specifically binds to a payload (drug) portion of an ADC. The PBA may be an antibody, a fragment or modification thereof, a peptide, an aptamer, a Spiegelmer, a fibronectin, a DARPin, a cyclodextrin, or an affitin. When the PBA is an antibody, the PBA may be referred to herein as an anti-drug antibody or anti-payload antibody. The anti-payload antibody may be whole immunoglobulin molecules such as polyclonal or monoclonal antibodies or chimeric antibodies including humanized antibodies or may be antigen-binding fragments thereof, including, but not limited to, Fab, F(ab′), F(ab′)2, Fv, dAb, Fd, CDR fragments, single-chain antibodies (scFv), bivalent single-chain antibodies, single-chain phage antibodies, diabodies, single domain antibodies (nanobodies) and the like. The fragments of the antibodies may be produced synthetically or by enzymatic or chemical cleavage of intact immunoglobulins or may be genetically engineered by recombinant DNA techniques. These techniques are well known in the art.

The term “chimeric antibody” refers to an antibody that has framework residues from one species, such as human, and complementarity-determining regions (CDRs, (which generally confer antigen binding) from another species, such as a murine antibody that specifically binds a payload. In a chimeric antibody, some portions of the heavy and/or light chains may be identical or homologous to sequences from a particular species while other portions may be identical or homologous to sequences from a different species. Chimeric antibodies generally exhibit decreased immunogenicity and increased stability. Techniques for cloning murine immunoglobulin variable domains known in the art—such as, for example, see Orlandi et al., Proc. Natl Acad. Sci. USA 86: 3833 (1989), and Leung et al., Hybridoma 13:469 (1994). As an example of a chimeric antibody, polynucleotides encoding the variable domains of the light chain or the heavy chain of an antibody derived from an animal (e.g., mouse, rat, or chicken) other than human can be linked to polynucleotides encoding the constant domains of the light chain or the heavy chain derived from a human antibody to produce a polynucleotide (such as DNA) encoding a chimeric antibody.

A “human” antibody (also called a “fully human” antibody) is an antibody that includes human framework regions and all of the CDRs from a single or different human immunoglobulins. Thus, frameworks from one human antibody can be engineered to include CDRs from a different human antibody. Methods for producing human antibodies are known in the art—such as, for example, see Mancini et al., 2004, New Microbiol. 27:315-28; Conrad and Scheller, 2005, Comb. Chem. High Throughput Screen. 8:117-26.

A “humanized antibody” is typically a human antibody that has one or more amino acid residues imported into it (i.e., introduced into it) from a source that is non-human. For example, a humanized antibody is a recombinant protein in which the CDRs of an antibody from a species such as rodent, rabbit, dog, goat, or horse are imported into human heavy and light variable domains. The constant domains (also referred to as framework regions) of the antibody molecule are generally the same as those of a human antibody. The non-human immunoglobulin providing the CDRs can be termed as “donor” and the human immunoglobulin providing the framework can be termed as “acceptor”. For example, all the CDRs can be from the donor immunoglobulin in a humanized immunoglobulin. Constant regions need not be always present, but if they are, they can be substantially identical to human immunoglobulin constant regions, i.e., at least about 85-90%, such as about 95% or more identical. A humanized antibody binds to the same payload as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions that have substantially no effect on antigen binding or other immunoglobulin functions. Humanized immunoglobulins can be constructed by means of genetic engineering (see for example, U.S. Pat. No. 5,585,089, and U.S. Publication No. 2010/0196266). For example, murine monoclonal antibodies may be isolated or generated and then humanized.

Antibody fragments can be produced by enzymatic digestion. For example, papain digestion of antibodies produces two identical antigen-binding fragments, called “Fab” fragments, and a “Fc” fragment. The Fab fragment contains an entire L chain and the variable region domain of the H chain (VH), and the first constant domain of one heavy chain. Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is capable of cross-linking antigen. “Fv” is the minimum antibody fragment that contains a complete antigen-recognition and -binding site and single-chain Fv also abbreviated as “sFv” or “scFv” are antibody fragments that comprise the VH and VL antibody domains connected into a single polypeptide chain. The term “diabodies” refers to small antibody fragments prepared by constructing sFv fragments with short linkers between the VH and VL domains such that inter-chain but not intra-chain pairing of the V domains is achieved, resulting in a bivalent fragment, i.e., fragment having two antigen-binding sites. A single domain antibody (sdAb) is an antibody fragment which has a single monomeric variable antibody domain. sdAbs can be made from heavy-chain antibodies found in camelids. An antibody fragment can be a single variable region or a peptide consisting of or comprising a single CDR. A single-chain antibody has a heavy chain variable domain and a light chain variable domain linearly linked to each other via a linker. A polynucleotide (such as DNA) encoding the single-chain antibody can be produced by binding a polynucleotide encoding the heavy chain variable domain, a polynucleotide encoding the linker (typically 10-20 nucleotides), and a polynucleotide encoding the light chain variable domain, with the heavy chain variable domain and the light chain variable domain being both derived from a human antibody.

The antibodies useful for the present method may be obtained from a human or a non-human animal. The antibody may be of any class (for example, IgG, IgE, IgM, IgD, IgA and IgY), or any subclass (e.g., IgG1, IgG2, IgG3, IgG4, IgA1 and IgA2). In an embodiment, single domain antibodies or nanobodies produced by camelids in response to introducing APP cleavage products (or peptide fragments thereof) into the camelids can be used. The nanobodies are typically heavy chain antibodies and thus contain heavy chain homodimers and do not contain antibody light chains. These antibodies typically comprise a single variable domain and two constant domains (CH2 and CH3).

The present disclosure also provides sequences that have homology with the protein or peptides sequences (including antibody sequences) described herein. In various examples, the homologous sequences have at least 85%, 86%, 87%, 88%, 89%, 90%, 91%, 92%, 93%, 94%, 95%, 96%, 97%, 98%, or 99% identity with a protein or peptide sequence of the present disclosure.

The payload molecule may be a drug molecule that causes cell toxicity. For example, any molecule that is used for treatment of cancer may be used. Examples include compounds, DNA, RNA, peptides and the like. In an embodiment, the drug molecule may be covalently bound to an N-terminal amino acid of the antibody via a reactive group or via a linker. Examples of reactive groups that cross link with the N-terminal alpha amine group of the antibody (such as light or heavy chain) include isothiocyanate, isocyate, acyl azide, NHS ester, sulfonyl ester, aldehyde, glyoxal, epoxide, carbonate, aryl halide, imidoester, carbodiimide, anhydride, fluorophenyl ester and the like. The reactive groups aldehyde or NHS ester are commonly used. Reduced cysteines (free sulfhydryls) may also be used. Conjugation methods of payload molecules to antibody are known. For example, conjugation of payload molecules to antibody is described in U.S. Pat. No. 10,071,170, the description of which is incorporated herein by reference.

Examples of payload molecules include microtubule formation inhibitors, meiosis inhibitors, topoisomerase inhibitors, RNA polymerase inhibitors, DNA intercalators or alkylators, ribosome inhibitors, siRNA, enzymes (carboxypeptidase, alkaline phosphatase, cytosine deaminase), immunocytokines (e.g. Interleukin-2) and the like. Examples of cytotoxic drugs include, but are not limited to, maytansinoid, auristatin, dolastatin, tubulysin, camptothecin, pyrrolobenzodiazepines, calicheamicin, gelonin, doxorubicin, duocarmycin, carboplatin, cisplatin, cyclophosphamide, ifosfamide, nidran, bleomycin, mitomycin C, cytarabine, fluorouracil, methotrexate, trimetrexate, vinblastine, alimta, altretamine, procarbazine, taxol, taxotere, diphtheria toxin, Pseudomonas exotoxin and derivatives (e.g. PE38, PE40), alpha-emitters (Ac-225, At-211, Th-227, Ra-223, Pb-212, Bi-212, Ra-224). Compounds include their stereoisomers and derivatives. In embodiments, the presently provided antibodies or antigen binding fragments thereof bind with specificity to Auristatin, which may be monomethyl auristatin E (MMAE) or monomethyl auristatin F (MMAF).

In various embodiments, the ADC, which includes conjugates of antibodies with cytotoxic agents, may comprise any antibody or a fragment or modification thereof that is useful against a tumor antigen or that is useful for delivering the cytotoxic drug to the tumor cells. For example, there are several monoclonal antibodies that have been demonstrated as successful therapeutic agents for the treatment of human cancers. These include rituximab, trastuzumab, cetuximab, panitumumab, bevacizumab and many others. Examples of monoclonal antibodies indicated against solid tumors include pertuzumab, ramucirumab, nivolumab, pembrolizumab, necitumumab, dinutuximab, olaratumab, atezolizumab, avelumab, cemiplimab, carotuximab, margetuximab, bemarituzumab, naxitamab, relatlimab, brentuximab, lorvotuzumab, glembatumumab, BCD-100, spartalizumab, IBI308, CS1001, tremelimumab, TSR-042). Any of these antibodies may be used to make an antibody drug conjugate.

Examples of antibodies and ADCs that are pertinent to this disclosure include, but are not limited to, gemtuzumab ozogamicin, brentuximab vedotin, trastuzumab emtansine (TDM1), inotuzumab ozogamicin, polatuzumab vedotin, trastuzumab deruxtecan, trastuzumab duocarmazine, sacituzumab govitecan, loncastuximab tesirine, oportuzumab monatox, zolbetuximab claudiximab, depatuxizumab mafodotin, mirvetuximab soravtansine, rovalpituzumab tesirine, enfortumab vedotin, ladiratuzumab vedotin, zilovertamab vedotin, tisotumab vedotin, disitamab vedotin, BAT8001, L191L2, and L19TNF.

In embodiments when the PBA is an antibody, the anti-drug antibody can be directed to the toxin (also referred to herein as the drug or payload) part of the ADC. In embodiments, the anti-drug antibody can be a fragment of the whole antibody. The fragment of the anti-drug antibody may be Fab, a Fab′, a F(ab′)2, a Fv, a scFv, a single domain antibody, or a diabody, or any other epitope binding fragment. The fragment of the antibody can be in the range of 0.5 kDa-110 kDa (Note: F(ab′)2 is ~100 kDa), including all Da values and ranges therebetween. In an embodiment, the antibody fragment is about 15 kDa. In an embodiment, the antibody is a single domain antibody (nanobody) containing only V_{H}H (generally, 13 kDa to 15 kDa). The anti-drug antibody has a binding affinity for the drug that may be expressed in terms of dissociation constant (K_{D}). In an embodiment, the K_{D} of the anti-drug antibody is from 1 pM to 50 nM, including all 0.1 pM values and ranges therebetween. In an embodiment, the K_{D} is less than 1 nM. In an embodiment, the K_{D} is from 1 pM to 100 pM. In an embodiment, the K_{D} can be from 1-100 pM. In an embodiment, the anti-drug antibody is a Camelid, chimeric, or humanized single domain antibody—also termed as a nanobody. These antibodies possess many characteristics that are ideal for the present competitive inhibition approach. Single domain antibodies are a small antibody format (~15 kDa), are highly stable, can be expressed in E. coli and can be humanized to limit immunogenicity. Additionally, camelid immunization and phage display technologies allow rapid and inexpensive development of novel inhibitors.

The antibody (including fragments or modifications) may bind to the free payload or payload cleaved from the ADC. When the payload is cleaved from the ADC, such as via enzymatic action, hydrolysis, oxidation, or some other mechanism, the cleaved payload may comprise a portion of the linking group, all of the linking group, or none of the linking group. The cleaved payload may further comprise substituents that may be added during or after cleavage, or functional groups formed as a result of the cleavage process. Examples of substituents and/or functional groups formed from the cleavage process include, but are not limited to, alkyl groups (e.g., methyl, ethyl, propyl, isopropyl, and the like), alcohol groups, amines, thiols, sulfonic acids, sulfoxides, sulfides, sulfones, carboxylic acids, esters, amides, and the like, and combinations thereof.

Multiple antibodies (including fragments or modifications) are described in the examples herein, and sequences are disclosed. Full-length antibodies, scFv, Fab and others, directed specifically to auristatins, including derivatives are included within the disclosure. Generation of the antibodies, screening of the antibodies to identify the candidates with the desired binding specificity and affinity, and their binding and inhibitory characteristics are described.

The sequences of any of the antibodies or fragments described herein may include a polyhistidine tag, or a linker, and the sequences may be used without the polyhistidine tag (e.g., to reduce immunogenicity), and when a linker is used the binding construct is not limited to the specific linker. Any specific sequences disclosed here with polyhistidine tags also include the corresponding sequences without the polyhistidine tags, and any sequences disclosed herein without the polyhistidine tags also include sequences with the polyhistidine tags. Variants of the sequences of antibodies or fragments disclosed herein include sequences which have at least 85% identity with the disclosed sequences providing the binding affinity is not adversely affected. For example, the binding affinity of variants may be 10% less, the same, or better than the disclosed sequences. In embodiments, the variants may have at least 90%, at least 95%, at least 98%, at least 99% homology (identity) with the disclosed sequences without adversely affecting the binding affinity. The disclosure also includes nucleotide sequences that encode for the amino acid sequences, or their variants as described herein.

The disclosure includes all polynucleotides that encode the described antibodies and antigen binding fragments thereof. Such polynucleotides include expression vectors that comprise the coding polynucleotides. The disclosure includes cell cultures that comprise the expression vectors, and methods of making the described antibodies and antigen binding fragments thereof by producing the antibodies or antigen binding fragments using the cell cultures, and separating the antibodies or antigen binding fragments from the cell cultures. Bacterial and mammalian cell cultures are included. The disclosure also includes all polynucleotides that can hybridize to a polynucleotide encoding a described antibodies or antigen binding fragments. The hybridization can be such that hybridization occurs in a solution at a temperature of about 20 degrees C. The solution may comprise a salt, such as a sodium salt, which may be at a concentration of about 0.15 M.

The PBA may be a peptide comprised of ten or more amino acids, and with an equilibrium dissociation constant (K_{D}) for binding to the payload that is 50.0 nM or lower.

It was surprising that a binding agent, such as an antibody, directed to the payload (drug) portion of the ADC such as MMAE does not adversely affect its efficacy with respect to cytotoxicity for cancer cells, but is able to reduce toxicity associated with the unconjugated payload. While not intending to be bound by any particular theory, it is possible the entry and intra-cellular processing of an ADC may be unaffected by binding to payload binding agents, and that the cellular entry of unconjugated payload is affected (reduced) by binding to a payload binding agent. However, the ability of payload binding agents to reduce toxicity of unconjugated payload relative to the anti-cancer cytotoxicity of ADCs, thereby enabling enhanced anti-cancer selectivity, is unexpected. In an embodiment, the potency of ADC may increase when used in conjunction with a PBA (such as an antibody), In such a case, not only does the non-target toxicity reduce, but the ADC efficacy is unexpectedly enhanced. In some embodiments, the PBA may not bind to the drug when it is a part of the ADC. Rather, it may bind to only free drug. In this case, it is expected there would be little or no impact on ADC efficacy, but a significant reduction in non-target toxicity.

The present disclosure provides a composition for reducing off-target toxicity comprising means for reducing toxicity of an unconjugated or cleaved payload (e.g., drug) from an ADC while not adversely affecting the efficacy of the ADC in the intended treatment (such as treatment for cancer cells). The means for reducing off-target toxicity includes anti-drug antibodies and fragments and modifications thereof, a peptide, an aptamer, a Spiegelmer, a fibronectin, a DARPin, a cyclodextrin, and/or an affitin.

In a non-limiting embodiment, a binding agent of this disclosure specifically binds to MMAE and includes a light chain and a heavy chain that comprise the following sequences:

ABC3319 Sequence
Light chain (H55Y Mutation in bold)
(SEQ ID NO: 3)
DIVMTQSPSSLSASVGDRVTITCRASQDVGTAVAWYQQKPGQSPKLLIYWASTRYTG
VPDRFSGSGSGTDFTLTISGLQSEDEADYFCQQYSNYPYTFGGGTKLEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Heavy chain (F27L mutation in bold, albumin
binding sequence underlined)
(SEQ ID NO: 4)
EVQLVESGGGLVKPGGSLKLSCAASGLTFSGYAMSWFRQAPEKRLEWVATISSGGSY
TYYLDSVKGRFTISRDNSKNTLYLQMSSLRSEDTAVYYCLASLITTDYFEYWGQGTLV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSC

In an embodiment, the ABC3319 (or any other binding partner of this disclosure) may be adapted to bind to a substance within blood in an individual, such as albumin or a blood cell surface protein, to increase its half-life. A non-limiting example of such a binding partner that binds to human serum albumin, which is an example of a substance within an individual, (and also specifically binds to MMAE) is referred to herein as ABC3320, wherein the light and heavy chains comprise the following sequences:

Light chain (H55Y Mutation in bold)
(SEQ ID NO: 3)
DIVMTQSPSSLSASVGDRVTITCRASQDVGTAVAWYQQKPGQSPK
LLIYWASTRYTGVPDRFSGSGSGTDFTLTISGLQSEDEADYFCQQ
YSNYPYTFGGGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

### Heavy Chain (F27L Mutation in Bold)

EVQLVESGGGLVKPGGSLKLSCAASGLTFSGYAMSWFRQAPEKRLEWVATISSGGSY TYYLDSVKGRFTISRDNSKNTLYLQMSSLRSEDTAVYYCLASLITTDYFEYWGQGTLV TVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKKVEPKSCGGGSRLIED ICLPRWGCLWEDD (SEQ ID NO:5). In this example, the human serum albumin binding domain is shown in bold and italicized font. An optional linker sequence, which may be substituted with any other suitable linker sequence, is shown in italics. In embodiments, a suitable linker may be 3-20 amino acids and may comprise G, S, or a combination thereof.

In embodiments any binding partner described herein, including but not necessarily limited to the binding partners referred to herein as ABC3319, ABC3320, and ABC3315, that contain amino acid residues at the unchanged positions shown in Table 1 (i.e., in L_H55Y the H is an unchanged position and Y is it's changed position in the light chain) may comprise any one or combination of amino acid substitutions (i.e., mutations) shown in Table 1. As noted above, the location of the point mutants shown in Table 1 is applicable to the sequence of any binding partner light and heavy chain having the stated amino acid at the same position in a binding partner as ABC3315. In Table 1 the prefix “L_” means light chain and the prefix “H_” means heavy chain. The same applies Mutatis mutandis to all other amino acid changes described herein.

TABLE 1
Kinexa Binding Analysis of ABC3315 Point Mutants

| ABC3315 Point | kon | koff | Dissociation | KD |
|---|---|---|---|---|
| Mutants | (10⁹M⁻¹h⁻¹) | (h⁻¹) | T_{1/2} (h) | (pM) |
| L_H55Y | 0.83 | 0.085 | 8.13 | 102.4 |
| L_R24H | 1.01 | 0.14 | 5 | 140 |
| H_F27L | 1.58 | 0.094 | 7.37 | 59 |
| H_L45P | 1.3 | 0.71 | 0.97 | 548 |
| H_E42D | 1.37 | 0.091 | 7.6 | 66 |
| F27L-H55Y | 1.51 | 0.024 | 27.34 | 15.9 |
| F27L-R24H | 1.22 | 0.146 | 4.75 | 119 |
| L45P-R24H | 1.87 | 0.26 | 2.7 | 139 |

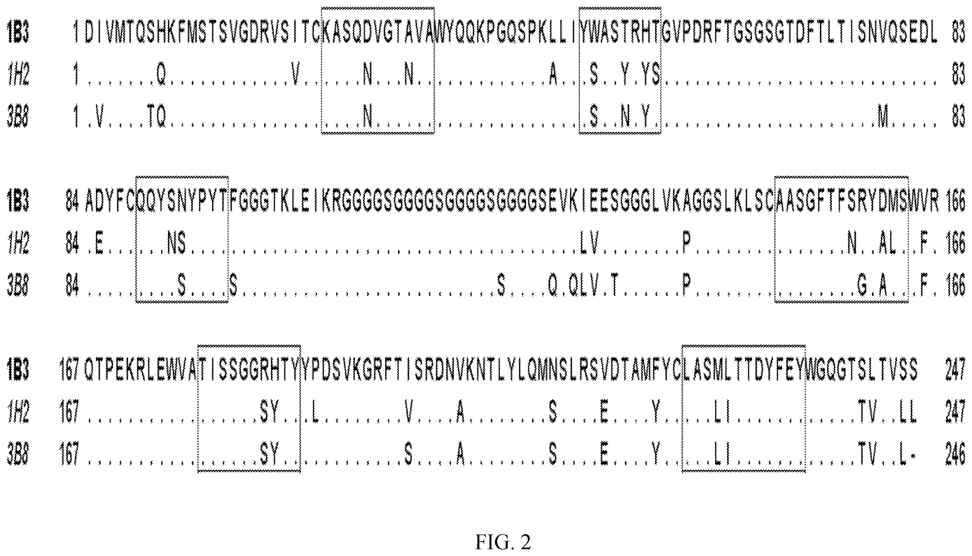

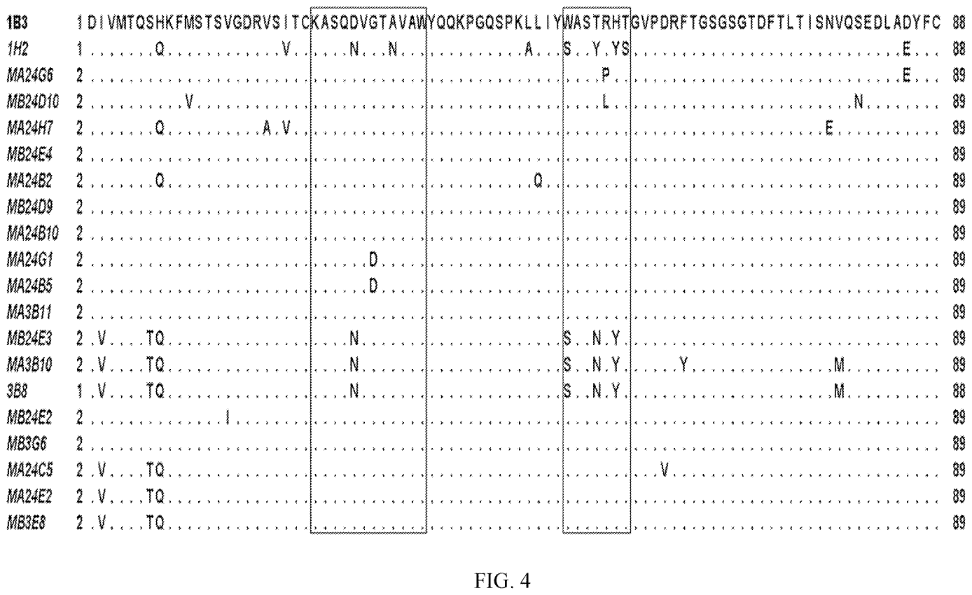

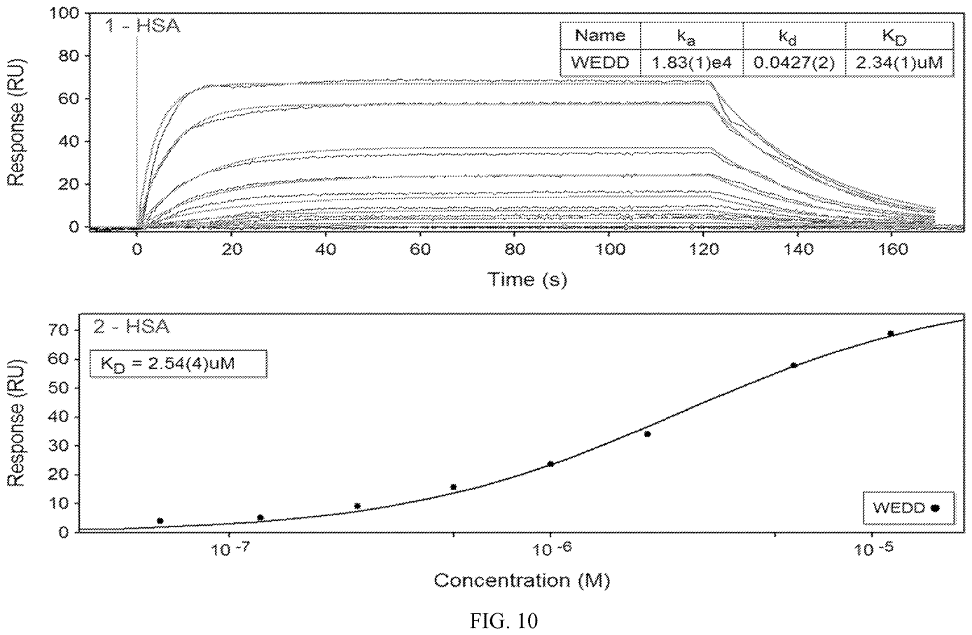

Another amino acid change that can be used alone or in combination with those described in Table 1 includes a I101F change in the heavy chain. As such, the disclosure expressly includes all binding partners that include one or more of those amino acid substitution, and all sequences that comprise or consist of any one or any combination of the described substitutions. In non-limiting embodiments, a binding partner of this disclosure includes only one or only a combination of amino acid substitutions shown in FIGS. 2, 4 and 12. As such, the disclosure expressly includes all binding partners that include one or more of those amino acid substitution, and all sequences that comprise or consist of any one or any combination of the described substitutions. Further, the amino acid sequences of each of the described binding partners that comprise any one or any combination of amino acid change shown in FIG. 2, FIG. 4, FIG. 12, and Table 1, are each expressly included within this disclosure. However, as discussed herein, a VH_F37V clone showed a markedly decreased binding signal relative to ABC3315. Thus, in embodiments, any binding partner described herein may exclude a mutation of F37 in its heavy chain.

As noted above, in embodiments, the PBA may be engineered for binding to formed elements within blood (e.g., albumin, red blood cell membrane proteins, etc.). PBA that have been engineered for binding to formed elements in blood may be expected to demonstrate reduced clearance (i.e., enabling greater convenience in dosing) and increased blood:tumor exposure, enabling greater pharmacokinetic selectivity (i.e., enabling greater inhibition of released payload molecules in blood relative to inhibition of released payload molecules within tumors). In embodiments, a component of a described binding agent comprises an amino acid sequence that binds to albumin, further representative and non-limiting examples of which include the following sequences: QRLIEDICLPRWGCLWEDDF (SEQ ID NO:6); QRLMEDICLPRWGCLWEDD (SEQ ID NO:7); QRLMEDICLPRWGCLWE (SEQ ID NO:8); and DICLPRWGCL (SEQ ID NO:9).

In an embodiment, a binding partner described herein is adapted to bind to a red blood cell. The adaptation may be in the form of another binding partner that becomes a component of an anti-payload agent. In embodiments, a binding partner of this disclosure thus comprises an amino acid sequence that imparts to the binding partner the ability to bind to a red blood cell. In embodiments, the red blood cell binding component comprises a single domain antibody.

In an aspect, the disclosure provides pharmaceutical compositions comprising or consisting essentially of the ADCs and the anti-payload agents as described herein. The formulations typically contain physiologically acceptable carriers, excipients, or stabilizers and may be in the form of aqueous solutions, lyophilized or other dried or solid formulations. Examples of suitable pharmaceutical preparation components can be found in Remington: The Science and Practice of Pharmacy 22th edition (2012). Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, polyethylene glycol (PEG) and the like. In an embodiment, the pharmaceutical composition may comprise buffer components and stabilizers, including, but not limited to, sucrose, polysorbate 20, NaCl, KCl, sodium acetate, sodium phosphate, arginine, lysine, trehalose, glycerol, and maltose. In embodiments, the ADCs and the anti-payload antibodies or fragments or modifications thereof are the only protein molecules present in the compositions. In embodiments, the ADCs and the anti-payload antibodies or fragments or modifications thereof, are the only antibodies present in the composition.

Compositions comprising the ADC and the payload binding agent may be administered together, or separately and independently using any suitable route including parenteral, subcutaneous, intraperitoneal, intrapulmonary, and intranasal. Parenteral infusions include intramuscular, intravenous, intraarterial, intraperitoneal, or subcutaneous administration. The administration(s) may be carried out in a continuous manner or may be intermittent. Appropriate dosage will depend upon the particular tumor being treated, the specifics and condition of the individual patient, the mode of administration etc. Determination of appropriate dosage is within the purview of one skilled in the art, such as a treating physician. In an embodiment, the ADC may be delivered locally, while the PBA is delivered so as to be available systemically. For example, and ADC may be delivered at or near the site of a tumor or intraperitoneally, while the PBA may be delivered i.v. In an embodiment, the ADC may be delivered systemically, and the anti-payload antibody is delivered so as to be available systemically. For example, an ADC may be delivered i.v., while the anti-payload antibody may be delivered subcutaneously.

The ADC and the PBA may be administered as a single composition or may be administered as separate compositions. When administered as separate compositions, they may be administered sequentially or concurrently. The two compositions may be administered at the same or different times, by the same or different routes, for same or different lengths of time, on the same or different regimens.

In an embodiment, the amount of the ADC and the PBA separately or together are in amounts sufficient enough to reduce the non-target toxicity of the ADC by at least 5% relative to that expected with the ADC alone at the same concentration as used in the combination. In embodiments, the reduction in toxicity may be 10%, 20%, 30%, 40%, 50% or more. Reduction in non-target toxicity can be evaluated by methods known in the art. For example, reduction could be classified as a reduction in the percent of patients at a specific dose that experience a grade 3 or higher adverse reaction with and without the PBA (Clin. Invest. (2013) 3(12), 1157-1165). Additionally, within an individual patient, reduction in non-target toxicity could be classified as a reduction in the severity of an adverse reaction (e.g., neutropenia) with, versus without the PBA.

The ADC and the PBA can be administered to an individual in need of treatment at dose(es) that is/are effective to treat a solid tumor. In general, suitable dosages of the PBA and the ADC can range from about 0.1 mg/kg to 100 mg/kg, including all 0.1 mg/kg values and ranges therebetween. Examples of dosages include 0.1, 0.5, 1, 5, 10, 15, 20, 25, 30, 35, 40, 45, 50, 55, 60, 65, 70, 75, 80, 85, 90, 95 and 100 mg/kg. A variety of dosage regimens are contemplated including dosage regimens in which the ADC and the PBA may be administered repeatedly, e.g., on a daily, weekly or monthly schedule, over a short period or an extended period of time, e.g., months to years (e.g., maintenance therapy). The range for administration of the PBA may be from 0.01-100 mg/kg, including all 0.01 mg/kg values and ranges therebetween. A suitable ratio of the ADC to the PBA may be determined by one skilled in the art. For example, a molar ratio can be from 1:1 to 1:100 ADC:PBA.

The amount of ADC and the anti-payload antibody can be administered to an individual in need of treatment at a dose that is effective to treat a solid tumor. In general, suitable dosages of the antibodies or fragments thereof—for both the ADC and the anti-payload antibody—can range from about 0.1 mg/kg to 100 mg/kg, including all 0.1 mg/kg values and ranges therebetween. Examples of dosages include 0.1, 0.5, 1, 5, 10, 15, 20, 25, 30, 35, 40, 45, 50, 55, 60, 65, 70, 75, 80, 85, 90, 95 and 100 mg/kg. A variety of dosage regimens are contemplated including dosage regimens in which the ADC and the anti-payload antibody may be administered repeatedly, e.g., on a daily, weekly or monthly schedule, over a short period or an extended period of time, e.g., months to years (e.g., maintenance therapy). The range for administration of the anti-payload antibody may be from 0.01-100 mg/kg, including all 0.01 mg/kg values and ranges therebetween. A suitable ratio of the ADC to the anti-drug antibody may be determined by one skilled in the art. In an embodiment, the molar ratio can be from 1:1 to 1:100 ADC:anti-Drug antibody.

As discussed above, in embodiments, half-life extension strategies can be used to increase plasma half-life of the anti-payload antibodies including fusion of moieties to the sdAb or Fab that bind blood components such as albumin, red blood cells (e.g., Band 3 of RBCs) or endogenous IgG as well as PASylation and PEGylation. Thus, in embodiments, the anti-payload antibody may be fused to moieties that bind to albumin, red blood cells or endogenous IgG, or fragments thereof, or may be PASylated and/or PEGylated. As an example, a bispecific antibody may have an arm that binds to the payload and another arm that binds to albumin or red blood cells (e.g., Band 3 of RBCs). In an embodiment, the antibodies or fragments do not contain a polyhistidine tag. It is therefore considered that sdAb-sdAb fusion proteins that combine anti-payload binding activity and anti-albumin or anti-red cell binding activity may exhibit desirable pharmacokinetic attributes (restricted distribution within tissues and tumors relative to distribution within blood, elimination via renal filtration due to low molecular weight, long half-life) and can provide optimal enhancement of ADC therapeutic selectivity (increasing the ratio of efficacy to off-site toxicity). It is also considered that sdAb-peptide or Fab-peptide fusion proteins that combine anti-payload binding affinity (via the sdAb or Fab) and albumin binding affinity (via the peptide) may exhibit desirable pharmacokinetic attributes (restricted distribution within tissues and tumors relative to distribution within blood, elimination via renal filtration due to low molecular weight, long half-life) and can provide optimal enhancement of ADC therapeutic selectivity (increasing the ratio of efficacy to off-site toxicity).

Representative examples of single domain antibodies that have been demonstrated to bind to red blood cells via Band 3 are as follows:

>RMC1
(SEQ ID NO: 10)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYKYRT
YDYWGQGTQVTVSS
>RA8
(SEQ ID NO: 11)
QVQLVQSGGGLVQAGGSLRLSCAASERTFSTYAMGWFRQIPGKERLFVAAVNWN
GKTIRYADSVKGRFTISRDNAKNTIALQMNSLKPEDTAVYYCALRSTPMYFTNL
ASQESYNYWGPGTQVTVSS
>RB12
(SEQ ID NO: 12)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYEYRT
YDYWGQGTQVTVSS
>RD1
(SEQ ID NO: 13)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYEYRT
YDYWGQGTQVTVSS
>RD11
(SEQ ID NO: 14)
EVQLVESGGGLVQPGGSLRLSCAASGRIFSISNMGWYRQAPGKQRELVATITSGGS
TNYGDSVKGRFTISMANAKNAVYLQMNSLKPEDTAVYYCNAGISRRTGTYSGGRYS
DYWAQGTQVTVSS
>RE8
(SEQ ID NO: 15)
QVQLVQSGGGLVQPGGSLRLSCAASGRVSEINTMGWYRQAPGKQRELVALITSTS
STMYSDSVKGRFTVSRDVAKNMVYLQMNSLKPEDTAVYYCNARQTKWYAAGYEY
RTYDYWGQGTQVTVSS
>RG10
(SEQ ID NO: 16)
QVQLQESGGGLVQAGGSLRLSCAASERTFSTYAMGWFRQTPGKERLFVAAVNWN
GKTIRYADSVKGRFTISRDNAKNTMSLQMNSLKPEDTAVYYCALRSTPMYFTNLASQ
ESYNYWGPGTQVTVSS
>RH5
(SEQ ID NO: 17)
QVQLVQSGGGLVQPGGSLRLSCAASGRIFSISNMGWYRQAPGKQRELVATITSGG
STNYGDSVKGRFTISMVNAKNAVYLQMNSLKPEDTAVYYCNAGISRRTGTYSGGRYS
DYWGQGTQVTVSS
>RMA1
(SEQ ID NO: 18)
QVQLVQSGGGLVQAGGSLRLSCAASGSSSSIIAMGWYRQAPGKQRELVATISNGD
TTNYTDSVKGRFTISRDKAKNTVYLQMASLEPEDTAVYYCNARQTKWYAAGYVYR
TYDYWGQGTQVTVSS
>RMD1
(SEQ ID NO: 19)
QVQLVQSGGGLVQPGGSLRLGCAASGRVSEINTMGWYRQAPGKQRGLVALITST
SSTMYSDSVKGRFTVSRDVAKNMVYLQMNSLKPEDTAVYYCNARLTRWYAAGYKY
RTYDYWGQGTQVTVSS
>RMF1
(SEQ ID NO: 20)
QVQLVQSGGGLVQAGGSLRLSCAASGSSSSIIAMGWYRQAPGKQRELVATISNGD
TTNYIDSVNGRFTISRDNAKNTVYLQMTSLEPEDTAVYYCHARQTKWYAAGYKYRT
YDYWGQGTQVTVSS
>RMG1
(SEQ ID NO: 21)
QVQLVQSGGGLVQPGGSLRLSCAASGRVSEINTMGWYRLAPGKQRELVALITSTS
STMYSDSVKGRFTVSRDVAKNMVYLQMNSLKPEDTAVYYCNARQTKWYAAGYKY
RTYDYWGQGTQVTVSS
>RMC2
(SEQ ID NO: 22)
QVQLVQSGGGLVQPGGSLRLSCAASGRVSEINTMGWYRQAPGKQRELVALITSTS
STMYSDSVKGRFTVSRDVAKNMVYLQMNSLKPEDTAVYYCNARQTIWYAAGYKYR
TYDYWGLGTQVTVSS
>RME2
(SEQ ID NO: 23)
QVQLVQSGGGLVQSGGSLRLSCAASGSSSSIIAMGWYRQAPGKQRELVATISNGD
TTNYIDSVKGRFTISRDNAKNTVYLQMASLEPEDTAVYYCNARQTKWYAAGYEYRT
YDYWGQGTQVTVSS
>RMF2
(SEQ ID NO: 24)
QVQLVQSGGGLVQAGGSLRLSCAASGSSSSIIAMGWYRQAPGKQRELVATISNND
TTNYIDSVKGRFTISRDNAKNTVYLQMASLEPEDTAVYYCNARQTKWYAAGYKYRT
YDYWGQGTQVTVSS
>RMG2
(SEQ ID NO: 25)
QVQLVQSGGGQVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYKYRT
YDYWGQGTQVTVSS
>RMH2
(SEQ ID NO: 26)
QVQLVQSGGGLAQAGGSLRLSCAASGSSSSIIAMGWYRQAPGKQRELVATISNGD
TTNYIDSVKGRFTISRDNAKNTVYLQMASLEPEDTAVYYCNARQTKWYAAGYKYRT
YDYWGQGTQVTVSS
>RMB3
(SEQ ID NO: 27)
QVQLVQSGGGLVQPGGSLRLSCAASGRVSEINTMGWFRQAPGKQRELVALITSTS
STMYSDSVKGRFTVSRDVAKNMVYLQMNSLKPEDTAVYYCNARQTKWYAAGYKY
RTYDYWGQGTQVTVSS
>RMD3
(SEQ ID NO: 28)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTRWYAAGYKYRT
YDYWGQGTQVTVSS
>RME3
(SEQ ID NO: 29)
QVQLVQSGGGLVQPGGSLRLSCAASGRVSEINTVGWYRQAPGKQRELVALFTSTS
STMYSDSVKGRFTVSRDVAKNMVYLQMNSLRPEDTAVYYCNARQTKWYAAGYKY
RTYDYWGQGTQVTVSS
>RMG3
(SEQ ID NO: 30)
QVQLVQSGGGLVQAGGSLRLSCAASGSSSSIIAMGWYRQAPGKQRELVATISNGD
TTNYIDSVKGRFTISRDNAKNTVYLRMASLEPEDTAVYYCNARQTKWYAAGYKYRT
YDYWGQGTQVTVSS

The present composition/compositions may be administered alone or in combination with other types of treatments (e.g., surgical resection, radiation therapy, chemotherapy, hormonal therapy, immunotherapy, or other anti-tumor agents).

The present compositions may be used for any type of cancer, including carcinoma, lymphoma, sarcoma, melanoma, and leukemia. Non-limiting examples include squamous cell cancer, small-cell lung cancer, non-small cell lung cancer, adenocarcinoma of the lung, squamous carcinoma of the lung, cancer of the peritoneum, myeloma (including multiple myeloma), hepatocellular cancer, gastrointestinal cancer, pancreatic cancer, glioblastoma/glioma (e.g., anaplastic astrocytoma, glioblastoma multiforme, anaplastic oligodendroglioma, anaplastic oligodendroastrocytoma), cervical cancer, ovarian cancer, liver cancer, bladder cancer, hepatoma, breast cancer, brain cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

In an aspect, this disclosure provides a method for generating and identifying anti-payload antibodies that are suitable to be used in combination with an ADC therapy to reduce non-target cytotoxicity. The method comprises generating or obtaining a library of antibodies, identifying specific antibodies based upon positive binding to the payload molecule by ELISA and/or by surface plasmon resonance techniques, identifying antibodies that have the desired affinity (such as K_{D} equal to or less than 50 nM, determining binding kinetic parameters, and determining in vivo efficacy. Any auristatin described herein may be a monomethyl auristatin.

In an embodiment, the present disclosure provides compositions and methods for treatment of cancer using ADC, where the drug portion of the ADC is an auristatin E (MMAE) or auristatin E derivative, analog, or metabolite, or auristatin F, or an auristatin F derivative, analog, or metabolite, or a related auristatin derivative. The method comprises administering to an individual in need of treatment an ADC, where the drug portion is an aforementioned auristatin, and a PBA directed to a described auristatin portion of the ADC. The ADC and the PBA may be administered in the same composition or in separate compositions as described elsewhere in this disclosure. An example of an ADC where the drug portion is auristatin E is brentuximab vedotin. Another example of an ADC where the drug portion is auristatin E is polatuzumab vedotin. When the drug portion of an ADC is auristatin E, an example of a PBA that can be used is IgM D9, sdAb MA3, MB2, and MC7. A method is also provided for the treatment of cancer (such as a solid tumor) comprising administering to an individual in need of treatment an ADC, wherein the drug is auristatin E and an antibody which may be a IgM D9, sdAb MA3, MB2, and MC7.

When the drug portion of an ADC is an auristatin examples of a PBA that can be used are described herein, and in the figures. In embodiments, the PBA is any one or a combination of ABC3320, ABC3319, ABC3317, ABC3315, 1B3, 1H2 3B8, 2E8, or 2C2.

In a non-limiting embodiments, the present disclosure is used with an ADC that is Polatuzumab Vedotin (PV).

Some non-limiting examples of PBA sequences provided by the present disclosure are as follows.

Sequences of clones identified from immunized
library screening
1B3
(SEQ ID NO: 31)
DIVMTQSHKFMSTSVGDRVSITCKASQDVGTAVAWYQQKPGQSPKLLI
YWASTRHTGVPDRFTGSGSGTDFTLTISNVQSEDLADYFCQQYSNYPYTFGGGTKLEI
KRGGGGSGGGGSGGGGSGGGGSEVKIEESGGGLVKAGGSLKLSCAASGFTFSRYDM
SWVRQTPEKRLEWVATISSGGRHTYYPDSVKGRFTISRDNVKNTLYLQMNSLRSVDT
AMFYCLASMLTTDYFEYWGQGTSLTVSS
1H2
(SEQ ID NO: 32)
DIVMTQSQKFMSTSVGDRVSVTCKASQNVGTNVAWYQQKPGQSPKA
LIYSASYRYSGVPDRFTGSGSGTDFTLTISNVQSEDLAEYFCQQYNSYPYTFGGGTKL
EIKRGGGGSGGGGSGGGGSGGGGSEVKLVESGGGLVKPGGSLKLSCAASGFTFNRYA
LSWFRQTPEKRLEWVATISSGGSYTYYLDSVKGRFTVSRDNAKNTLYLQMSSLRSED
TAMYYCLASLITTDYFEYWGQGTTVTVLL
3B8
(SEQ ID NO: 33)
DVVMTQTQKFMSTSVGDRVSITCKASQNVGTAVAWYQQKPGQSPKLL
IYSASNRYTGVPDRFTGSGSGTDFTLTISNMQSEDLADYFCQQYSSYPYTSGGGTKLE
IKRGGGGSGGGGSGGGGSSGGGSQVQLVETGGGLVKPGGSLKLSCAASGFTFSGYA
MSWFRQTPEKRLEWVATISSGGSYTYYPDSVKGRFTSSRDNAKNTLYLQMSSLRSED
TAMYYCLASLITTDYFEYWGQGTTVTVL
Sequences from mutagenesis library m = mutagenesis, A/B = plate,
3/24 = dissociation time, Letter# = Well (refer to Examples)
MA24G1
(SEQ ID NO: 34)
MDIVMTQSHKFMSTSVGDRVSITCKASQDVDTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSEVKLVESGGGLVKPGGSLKLSCAASGFTFNRYA
LSWFRQTPEKRLEWVATISSGGSYTYYLDSVKGRFTVSRDNAKNTLYLQMSSLRSED
TAMYYCLASLITTDYFEYWGQGTTVTVLL
MA24E2
(SEQ ID NO: 35)
MDVVMTQTQKFMSTSVGDRVSITCKASQDVGTAVAWYQQKPGQSPK
LLIYWASTRHTGVPDRFTGSGSGTDFTLTISNVQSEDLADYFCQQYSNYPYTFGGGTK
LEIKRCGGGSGGGGSGGGGSGGGGSQVQLVETGGGLVKPGGSLKLSCAASGFTFSGY
AMSWFRQTPEKRLEWVATISSGGSYTYYLDSVKGRFTVSRDNAKNTLYLQMSSLRSE
DTAMYYCLASLITTDYFEYWGQGTTVTVLL
MA24C5
(SEQ ID NO: 36)
MDVVMTQTQKFMSTSVGDRVSITCKASQDVGTAVAWYQQKPGQSPK
LLIYWASTRHTGVPVRFTGSGSGTDFTLTISNVQSEDLADYFCQQYSNYPYTFGGGTK
LEIKRGGGGSGGGGSGGGGSGGGGSQVQLVETGGGLVKPGGSLKLSCAASSSTFSGY
AMSWFRQTPEKRLEWVATISSGGSYTYYLDSVKGRFTVSRDNAKNTLYLQMSSLRSE
DTAMYYCLASLITTDYFEYWGQGTTVTVLL
MA24B5
(SEQ ID NO: 37)
MDIVMTQSHKFMSTSVGDRVSITCKASQDVDTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSEVKLVESGGGLVKPGGSLKLSCAASGFTFNRYA
LSWFRQTPEKRLEWVATISSGGSYTYYLDSVKGRFTVSRDNAKNTLYLQMSSLRSED
TAMYYCLASLITTDYFEYWGQGTTVTVLL
MA24H7
(SEQ ID NO: 38)
MDIVMTQSQKFMSTSVGDRASVTCKASQDVGTAVAWYQQKPGQSPK
LLIYWASTRHTGVPDRFTGSGSGTDFTLTISEVQSEDLADYFCQQYSNYPYTFGGGTK
LEIKRGGGGSGGGGSGGGGSGGGGSEVKLVESGGGLVKPGGSLKLSCAASGFTFNRY
ALSWFRQTPEKRLEWVATISSGGSYTYYLDSVKGRFTVSRDNAKNTLYLQMSSLRSE
DTAMYYCLASLITTDYFEYWGQGTTVTVLL
MA24G6
(SEQ ID NO: 39)
MDIVMTQSHKFMSTSVGDRVSITCKASQDVGTAVAWYQQKPGQSPKL
LIYWASTPHTGVPDRFTGSGSGTDFTLTISNVQSEDLAEYFCQQYNSYPYTFGGGTKL
EIKRGGGGSGGGGSGGGDSGGGGSEVKLVESGGGLVIPGGSLKLSCAASGFTFNRYA
LSWFRQTPEKRLEWVATISSGGSYTYYLDSVKGRFTVSRDIAKNTLYLQMSSLRSEDT
AMYYCLASLFTTDYFEYWGQGTTVTVPL
MA24B10
(SEQ ID NO: 40)
MDIVMTQSHKFMSTSVGDRVSITCKASQDVGTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSEVKLVESGGGLVKPGGSLKLSCAARGFTFNRY
ALSWFRQTPEKRLEWVATISSGGSYTYYLDSVKGRFTVSRDNAKNTLYLQMSSLRSE
DTAMYYCLASLITTDYFEYWGQGTTVTVLL
MA24B2
(SEQ ID NO: 41)
MDIVMTQSQKFMSTSVGDRVSITCKASQDVGTAVAWYQQKPGQSPKL
QIYWASTRHTGVPDRFTGSGSGTDFTLTISNVQSEDLADYFCQQYSNYPYTFGGGTK
LEIKRGGGGSGGGGSGGGGSGGGGSEVKLVESGGGLVKPGGSLKLSCAASGFTFNRY
ALSWFRQTPENRLEWVATISSGGSYTYYLDSVKGRFTVSRDNAKNTLYLQMSSLRSE
DTAMYYCLASLITTDYFEYWGQGTTVTVLL
MB24E2
(SEQ ID NO: 42)
MDIVMTQSHKFMSTSIGDRVSITCKASQDVGTAVAWYQQKPGQSPKLL
IYWASTRHTGVPDRFTGSGSGTDFTLTISNVQSEDLADYFCQQYSNYPYTFGGGTKLE
IKRGGGGSGGGGSGGGGSGGGGSQVQLVETGGGLVKPGGSLKLSCAASGFTFSGYA
MSWFRQTPEKRLEWVATISSGGSYTYYLDSVKGRFTVSRDNAKNTLYLQMSSLRSED
TAMYYCLASLITTDYFEYWGQGTTVTVLL
MB24D9
(SEQ ID NO: 43)
MDIVMTQSHKFMSTSVGDRVSITCKASQDVGTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSEVKLVESGGGLVKPGGSLKLSCAASGFTLNRYA
LSWFRQTPEKRLEWVATISSGGSYTYYLDSVKGRFTVSRDNAKNTLYLQMSSLRNED
TAMYYCLASLITTDYFEYWGQGTTVTVLL
MB24E4
(SEQ ID NO: 44)
MDIVMTQSHKFMSTSVGDRVSITCKASQDVGTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSEVKIEESGGGLVKAGGSLKLSCAASGFTFNRYA
LSWFRQTPEKRLEWVATISSGGSYTYYLDSVKGRFTVSRDNAKNTLYLQMSSLRSED
TAMYYCLASLITTDYFEYWGQGTTVTVLL
MB24E3
(SEQ ID NO: 45)
MDVVMTQTQKFMSTSVGDRVSITCKASQNVGTAVAWYQQKPGQSPK
LLIYSASNRYTGVPDRFTGSGSGTDFTLTISNVQSEDLADYFCQQYSNYPYTFGGGTK
LEIKRGGGGSGGGGSGGGGSGGGGSEVKLVESGGGLVKPGGSLKLSCAASGFTFNRY
ALSWFRQTPENRLEWVATISSGGSYTYYPDSVKGRFTSSRDNAKNTLYLQMSSLRSE
DTAMYYCLASLITTDYFEYWGQGTTVTVLL
MB24D10
(SEQ ID NO: 46)
MDIVMTQSHKFVSTSVGDRVSITCKASQDVGTAVAWYQQKPGQSPKL
LIYWASTLHTGVPDRFTGSGSGTDFTLTISNVQNEDLADYFCQQYSNYPYTFGGGTK
LEIKRGGGGSGGGGSGGGGSGGGGSEVKLVESGGGLVKPGGSLKLSCAASGFTFNRY
ALSWFRQTPEKRLEWVATISSGGSYTYYLDSVKGRFTVSRDNAKNTLYLQMSSLRSE
DTAMYYCLASLITTDYSEYWGQGTTVTVLP
MA3B10
(SEQ ID NO: 47)
MDVVMTQTQKFMSTSVGDRVSITCKASQNVGTAVAWYQQKPGQSPK
LLIYSASNRYTGVPDRYTGSGSGTDFTLTISNMQSEDLADYFCQQYSNYPYTFGGGTK
LEIKRGGGGSGGGGSGGGGSGGGGSEVKLVESGGGLVKPGGSLKLSCAASGFTFNRY
ALSWFRQTPEKRLEWVATISSGGSYTFYLDSVKGRFTVSRDNAKNTLYLQMSSLRSE
DTAMYYCLASMLTTDYFEYWGQGTTVTVLL
MA3B11
(SEQ ID NO: 48)
MDIVMTQSHKFMSTSVGDRVSITCKASQDVGTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSEVKLVESGGGLVKPGGSLKLSCAASGFTFNRYA
LSWFRQTPEKRLEWVATISSGGSYTYYLDSVKGRFTVSRDNAKNTLYLQMSSLRSED
TAMYYCLASLITTDYFEYWGQGTTVTVLL
MB3G6
(SEQ ID NO: 49)
MDIVMTQSHKFMSTSVGDRVSITCKASQDVGTAVAWYQQKPGQSPKL
LIYWASTRHTGVPDRFTGSGSGTDFTLTISNVQSEDLADYFCQQYSNYPYTFGGGTKL
EIKRGGGGSGGGGSGGGGSGGGGSQVQLVETGGGLVKPGGSLKLSCAASGFTFSGY
AMSWFRQTPEKRLEWVATISSGGSYSYYLDSVKGRFTVSRDNAKNTLYLQMSSLRSE
DTAMYYCLASLITTDYFEYWGQGTTVTVLL
MB3E8
(SEQ ID NO: 50)
MDVVMTQTQKFMSTSVGDRVSITCKASQDVGTAVAWYQQKPGQSPK
LLIYWASTRHTGVPDRFTGSGSGTDFTLTISNVQSEDLADYFCQQYSNYPYTFGGGTK
LEIKRGGGGSGGGGSGGGGSGGGGSQVQLVETGGGLVKPGGSLKLSCAASGFTFSG
YAMSWFRQTPEKRLEWVATISSGGSYTYYLDSVKGRFTVSRDNAKNTLYLQMSSLRS
EDTAKYYCLASLFTTDYFEYWGQGTTVTVLL
ABC3315 Humanized Fab Sequence (predicted CDR
sequences shown in bold)
Light Chain
(SEQ ID NO: 1)
DIVMTQSPSSLSASVGDRVTITCRASQDVGTAVAWYQQKPGQSPKLLI
YWASTRHTGVPDRFSGSGSGTDFTLTISGLQSEDEADYFCQQYSNYPYTFGGGTKLE
IKRTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Heavy Chain
(SEQ ID NO: 2)
EVQLVESGGGLVKPGGSLKLSCAASGFTFSGYAMSWFRQAPEKRLEW
VATISSGGSYTYYLDSVKGRFTISRDNSKNTLYLQMSSLRSEDTAVYYCLASLITTDY
FEYWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEPK
SC

The disclosure includes antibodies and antigen binding fragments thereof with all amino acid substitutions individually and in all combinations as shown in FIG. 4, including but not necessarily limited to amino acid substitutions designated within the boxes.

In an aspect, this disclosure provides kits comprising an ADC and components for reducing non-target toxicity of the ADC. The kits may comprise in the same or different composition, i) ADC, and ii) an anti-payload antibody, wherein the anti-payload antibody is specific for the drug portion of the ADC. The ADC and the anti-payload antibody may be provided in a powdered, lyophilized form, along with reconstitution media, where the antibody and the ADC can be reconstituted prior to use. The kit may also optionally comprise instructions for administration of the compositions comprising the ADC, and the composition comprising the anti-payload antibody, which may be administered via different routes.

Some non-limiting examples are provided in the following paragraphs.
Example 1. A method for reducing non-target toxicity of an antibody-drug conjugate (ADC) comprising administering to an individual in need of treatment the ADC and a payload binding agent (PBA) which is directed to the drug portion of the ADC. In various examples, the drug portion is a cleaved payload from an ADC.
Example 1a. The method of Example 1, wherein the PBA is an antibody of a fragment or modification thereof, which is directed to the drug portion of the ADC (anti-drug antibody).
Example 1b. The method of Example 1, wherein PBA is a peptide.
Example 2. The method of Example 1, wherein the ADC and the PBA are administered in the same composition.
Example 3. The method of Example 1, wherein the ADC and the PBA are administered in different compositions.
Example 4. The method of Example 3, wherein the ADC and the PBA are administered via different routes.
Example 4a. The method of Example 4, wherein the ADC is administered by intraperitoneal route and the PBA is administered intravenous route.
Example 5. The method of Example 1, wherein the drug is auristatin E or auristatin F.
Example 6. The method of Example 1, wherein the K_{D} of the PBA is less than or equal to 50 nM.
Example 6a. The method of Example 1, wherein the K_{D} of the anti-drug antibody is less than or equal to 50 nM.
Example 7. The method of Example 6 or 6a, wherein the K_{D} of the PBA (Example 6) or the anti-drug antibody (Example 6a) is less than 1 nM.
Example 8. The method of Example 7, wherein the K_{D} of the PBA or the anti-drug antibody is from 1 pM to 100 pM.
Example 9. A composition comprising an ADC and a PBA, which is directed to the drug portion of the ADC.
Example 9a. The composition of Example 9, wherein the PBA is an antibody
of a fragment or modification thereof, which is directed to the drug portion of the ADC (anti-drug antibody).
Example 10. A kit comprising: i) a composition comprising an ADC; ii) a composition comprising an anti-drug antibody which is specific for the drug in the ADC; iii) optionally, instructions for use including instructions for administration of i) and ii).

The invention is further demonstrated by way of the figures and data presented herein, whether provided in the detailed description or the figures.

### Example 1

This Example provides a description of compositions and methods used in the present disclosure, and particularly for identification of mouse antibodies against MMAE.

### Methods

### Immunization

Monomethyl auristatin F (MMAF) was conjugated to keyhole limpet hemocyanin (KLH) or BSA via an EDC linker. Briefly, 0.5 mg MMAF was dissolved in MES buffer (0.1 M MES, pH 4.7)/30% DMF and mixed with 2 mg of KLH or BSA in MES buffer, then 0.5 mg of EDC dissolved in water was added. The solution was incubated at room temperature overnight. Approximately 50 g of KLH-MMAF immunogen emulsified in Freund's incomplete adjuvant was used for single animal immunization. Female Balb/c mice were subcutaneously injected with 200 L of emulsion and given a booster every three weeks. Anti-MMAF-BSA plasma titers were evaluated with ELISA using an anti-mouse Fc secondary antibody conjugated to alkaline phosphatase.

### RNA Isolation

Spleens were surgically removed from immunized mice and teased apart with forceps and a needle. The released splenocytes were collected in a 15-ml conical tube. The lymphocyte samples were lyzed and homogenized in 5 ml of TRIzol reagent, then incubated for 5 minutes before adding 1 mL of chloroform. The mixture was vortexed for 30 seconds, incubated for 3 minutes, and centrifuged for 15 minutes at 12,000×g 4° C., after which the mixture was separated into 3 layers: a lower red phenol-chloroform, interphase, and a colorless upper aqueous phase. The aqueous phase containing RNA was transferred into a new tube, 2.5 ml of isopropanol was added, and incubated for 10 minutes. The sample was centrifuged for 10 minutes at 12,000×g 4° C., and total RNA precipitate formed a white gel-like pellet. The supernatant was discarded, and the pellet was resuspended in 5 ml of 75% ethanol. The sample was vortexed briefly, then centrifuged for 5 minutes at 10,000×g 4° C. The supernatant was discarded, and the pellet was air-dried for 15 minutes. The pellet was resuspended in 200 μL of RNAse-free water and incubated for 10 minutes at 60° C. to ensure complete solubilization of total RNA. The RNA sample was stored at −80° C. for subsequent procedures.

cDNA Synthesis

The following reagents were mixed, heated at 65° C. for 5 minutes, and then incubated on ice for at least 1 minute: 1 μl of 50 M Oligo d(T)20 primer, 1 μl of 10 mM dNTP mix (10 mM each), 2.5 μl of template RNA, and 9.5 μl of nuclease-free water. The following reagents were added to the RNA-primer mixture: 4 μl of 5×SSIV Buffer, 1 μl of 100 mM DTT, 1 μl of RNaseOUT™ Recombinant RNase Inhibitor, and 1 μl of SuperScript® IV Reverse Transcriptase (200 U/L). The combined reaction mixture was incubated at 50-55° C. for 15 minutes and then inactivated at 80° C. for 10 minutes.

DNA Amplification and scFv

Mouse variable heavy chain (VH) and light chain (VL) were amplified via polymerase-chain reaction (PCR) with the following reagents: 25 μl of OneTaqGC 2× master mix, 2 μl of cDNA, 0.5 μl of forward primers, 0.5 μl of reverse primers and 22 μl nuclease-free water. PCR products were obtained following 30-cycles of denaturation at 94° C. for 30 seconds, annealing at 55° C. for 30 seconds, and extension at 68° C. for 45 seconds with a final 5-minute extension. The amplified VH and VL products (~400 bp) were size selected on a 1% agarose gel and purified, and mouse scFvs were assembled with added SfiI cloning sites via overlap-extension PCR with the following reagents: 25 μl of OneTaqGC 2× master mix, 10 ng of purified VH and VL each, 1 μl of forward and reverse primers each, 5 μl of GC Enhancer, and water q.s 50 μl. PCR was performed with the following conditions: 5 cycles of denaturation at 95° C. for 1 minute, annealing at 63° C. for 1 minute, and extension at 72° C. for 1 minute; followed by 5 cycles of denaturation at 95° C. for 1 minute, annealing at 56° C. for 30 seconds, and extension at 72° C. for 1 minute; following with 25 cycles of denaturation at 95° C. for 1 minute, combined annealing and extension at 72° C. for 90 seconds with a final 5-minute extension. The amplified scFv products (~850 bp) were size-selected on a 1% agarose gel and purified.

### Immunized Phage-Displayed Libraries Construction

For test library construction, amplified PCR product (1 μg/replicate) and pADL-10b phagemid vector (2 μg/replicate) were digested in triplicate with SfiI restriction enzyme according to the manufacturer's recommendation. Reactions were incubated at 50° C. for 16 h. Cut products were recovered through DNA gel electrophoresis using a 1% agarose gel. A test library was first developed by ligating digested DNA into the phagemid vector. Ligated vector was purified with an E.Z.N.A cycle pure kit. Electrocompetent TG-1 cells were transformed by electroporation with 2 μL of DNA according to the following conditions: 1.0 mm cuvette, 10 pF, 600 Ohms, 1800 Volts, time constants 3.5 to 4.5 msec. Immediately after pulsating cells, 975 μL of Recovery Medium was added to the cells. The cell suspension was then transferred into a 3 mL tube and incubated at 250 rpm for 1 h at 37° C. The cell suspension was then serially diluted to 1:1000 on LB agar plates containing 100 g/ml ampicillin and 2% (wt/v) glucose and grown overnight at 37° C. Electroporation efficiency was determined by counting the colonies and multiplying by the corresponding dilution factor. Approximately 10-20 clones were isolated, re-suspended in water, and incubated at 95° C. for 5 minutes, followed by centrifugation at 4° C. for 5 min. DNA within the supernatant was amplified as described previously using 5′ and 3′ sequencing primers. PCR products were purified and sequenced to assess the functionality and size of the library.

For full library construction, enough DNA was ligated into pADL-10b to yield a library of at least 10⁷ individual transformants. Several separate electroporations were carried out using 3 μL of ligated DNA and 25 μL of TG1 cells. All electroporations were combined into a 50 mL conical tube. Transformed bacteria were serially diluted to 1:10,000 on LB agar plates containing 100 g/ml ampicillin and 2% (wt/v) glucose and grown overnight at 37° C. Library size was evaluated by colony counting. Fragmentation PCR and sequencing were conducted as described above. The remaining bacteria suspension was plated onto four 245 mm square LB agar dishes containing 100 g/ml ampicillin and 2% (wt/v) glucose and grown overnight at 37° C. This process was repeated until the library size was ≥10⁷. The library was recovered by first scraping bacteria from the 245 mm dishes in 8 mL of LB medium and combined into a 50 ml conical. The collected cell suspension was mixed with sterile glycerol (20%, v/v). Cell library aliquots were stored at −80° C.

A thawed library aliquot was used to inoculate 60 mL of 2×YT medium supplemented with 100 g/ml ampicillin and 2% (wt/v) glucose in a baffled 250 mL Erlenmeyer. The inoculated medium was grown at 37° C. until OD₆₀₀≈0.5. To rescue phage, 10 mL of broth was transferred to a 50 mL conical where 1 μL of CM13 helper phage was added and incubated for 1 h at 37° C. at 250 rpm. Infected cells were then isolated by centrifugation at 2,800×g for 10 minutes at room temperature. Cells were re-suspended in 50 ml of 2×YT supplemented with 100 μg/mL ampicillin and 50 μg/mL kanamycin into a baffled 250-ml Erlenmeyer flask incubated overnight at 30° C. and 200 rpm. Bacterial culture was transferred to two 50-mi conical tubes and centrifuged for 15 minutes at 3,200×g and 4° C. to pellet cells. Supernatants were isolated into new 50 mL conicals, where 6 mL of 20% (wt/v) PEG6000/2.5 M NaCl solution was added, mixed by inversion, and chilled on ice for 30 minutes. Phage particles were pelleted by centrifugation for 10 minutes at 3,200×g and 4° C. Precipitated phage particles were re-suspended in 1 mL of ice-cold PBS, and transferred to a microcentrifuge tube. Phage particle suspension was centrifuged for 90 seconds at 16,000×g and 4° C. to pellet any residual bacteria. Phage particles from the supernatant were re-precipitated by adding 250 μl of ice-cold 20% (wt/v) PEG6000/2.5 M NaCl solution, mixed by inversion, and incubated on ice for 10 minutes. Phage particles were pelleted by centrifugation at 16,000×g at 4° C. for 20 minutes and re-suspended in 0.5 mL of ice-cold PBS. The remaining bacterial debris was removed by centrifugation for 90 seconds at 16,000×g and 4° C. Phage concentration was determined before panning via a titration method. Briefly, phage was serially diluted by factors of 10 in PBS. To a single well on a low-binding 96-well round-bottom culture plate, 10 μL of each dilution to a well with 90 μL of TG1 cells (pre-grown suspension). Plates were incubated for 15 min at 37° C. to infect TG1 cells. Approximately 5 μL of the infected TG1 cells from each dilution was transferred to solid selective medium (LB+100 μg/mL ampicillin+2% glucose) and grown overnight at 37° C. Phage concentration is determined by counting colonies on the highest diluted plate that showed growth.

Biopanning and Screening Mouse scFvs

MMAF was conjugated to biotin-PEG2-amine and used for bio-panning and screening of anti-MMAE scFvs. 0.5 mg MMAF was dissolved in MES buffer (0.1 M MES, pH 4.7)/30% DMF and mixed with 100 μg of biotin-PEG2-amine in MES buffer, then 1.5 mg of EDC dissolved in water was added. The reaction was incubated overnight at room temperature and stored at 4° C. Streptavidin magnetic beads were washed three times with PBST (PBS+0.05% Tween 20) and blocked with MPBS (PBS+5% non-fat dry milk) for 2 h. After the blocking step, the streptavidin beads were incubated with 1 mL of 1 μM biotin-MMAF for 15 minutes, followed by three washes with PBST. For the first panning input, the stock phage was diluted to 10¹² c.f.u. (colony forming unit)/mL in blocking buffer (2% milk PBS), and 1 ml of the diluted phage was added to the MMAF-biotin coated streptavidin beads and incubated for 2 hours. For the subsequent panning, the stock phage was diluted 1:1 in blocking buffer. After the incubation, beads were washed with PBST 5 times, 10 times, 15 times, and 15 times for the 1st, 2nd, 3rd, and 4th round of panning. Bound phages were then eluted by incubation with free MMAE at the concentrations of 1 μM, 100 nM, 10 nM, and 1 nM in PBS over 1 hour for the 1st, 2nd, 3rd, and 4th round of panning. Output phages were titrated and re-infected with TG-1 cells for phage production of the next round of panning.

Following the fourth round of panning phage-infected TG1 cells were grown overnight, serially diluted in 2×YT media, spread over individual culture plates containing selective medium (LB agar+100 μg/mL ampicillin+2% wt/v glucose), and incubated overnight at 37° C. A master plate was generated by inoculating a single colony into wells of a 96-well round-bottom culture plate filled with 100 μL of 2×TY supplemented with 100 μg/mL ampicillin, 2% (wt/vol) glucose and 15% (vol/vol) glycerol, and grown overnight at 37° C., 300 rpm. These wells were then used to inoculate wells of 96 deep-well plates containing 1 mL of 2×TY medium (100 μg/mL ampicillin per well). Plates were incubated for 4 h at 37° C. and 300 rpm until OD₆₀₀≈0.5, then 1 μL of stock helper phage were added to each well and incubated for another 1 hour. Kanamycin was added to a final concentration of 50 μg/mL, and the plates were incubated overnight at 30° C. 250 rpm.

Nunc Maxisorp 96-well ELISA plates were coated with 4 g/ml NeutrAvidin overnight at 4° C. Plates were washed five times with PBST, blocked with MPBS for 2 hours at room temperature and 100 μL of 1 M Biotin-MMAF was added for 30 minutes. Plates were then washed five times with PBST and incubated with 4-fold diluted phage supernatant for 2 hours, with or without pre-incubation of free MMAE at concentrations of 10 nM and 100 nM, and with 100 nM of MMAE-ADC. Plates were then washed five times with PBST, and bound scFv-displaying phages were detected using an anti-M13 phage HRP-conjugated antibody (Antibody Design Labs, San Diego, CA) diluted 1:1000 in MPBST for 1-hour. Following five washes with PBST, 100 L of 1-Step Turbo TMB-ELISA solution was added to each well and incubated for 15 minutes. The reaction was quenched by adding 100 μL of Stop solution to each well, and absorbance was measured at 450 nm. DNA isolated from positive clones were sent to the Roswell Park sequencing core facility (Buffalo, NY) for sequencing.

### Mutagenesis Library

Genes for scFv clones 3B8, 1H2 and 1B3 were codon optimized for E. coli and synthesized by GeneArt. The heavy and light chains for each clone were amplified by PCR and subsequently mutated using a PCR based random mutagenesis method. The purified mutagenesis product was combined, and the heavy and light chains ligated using an overlap extension PCR method. The full library was constructed following the methods used to construct the immunized phage library with the phagemid vector pComb3XSS used in place of the pADL-10b phagemid vector.

### Mutagenesis Library Panning and Screening

Phage was panned against MMAF-peg11-biotin-streptavidin coated beads for three rounds, with increasing stringency. Following the third round of panning, phage displaying scFvs were dissociated with 1 μM of MMAE for 3 hours, supernatant removed, and remaining phage eluted with 1 μM MMAE for 24 hours. Two 96-deep well plates were inoculated with single colonies from the 24-hour elution and phage expressed in deep well plates following the protocol provided above. The following day, the phage containing supernatant was diluted 1:10 in 2% milk-PBS in individual wells of a 96-well plate with or without 1 nM free MMAE for 1-hour. Subsequently, the phage containing solution was transferred to the wells of a Nunc Maxisorb plate containing immobilized MMAF-peg11-biotin-neutravidin and incubated for 2-hours at room temperature on a shaking incubator. Plates were washed and bound phage detected. Colonies with a signal knockdown >70% were sent for DNA sequencing.

### Humanization

The amino acid sequence for the variable heavy and variable light chain for clone MA24E2 were input into Abysis. Murine framework residues that occur with low frequency in human antibodies were conservatively mutated to an amino acid with high frequency. The murine and humanized sequences were input into A bodybuilder to create a structural model prediction for the human and murine sequences. Predicted structures were overlayed in ChimeraX to ensure the human sequence was predicted to have a similar structure as the murine sequence. The humanized sequence was expressed as a fab fragment (ABC3315) in ExpiCHO-S cells and purified using CaptureSelect™ CH1-XL affinity resin following manufacturer recommendations.

### Competitive ELISA

Anti-MMAE fab was diluted to 1 nM and incubated with a range of concentrations of MMAE, MMAF and trastuzumab-vc-MMAE. Solutions were added in triplicate to individual wells of an ELISA plate with MMAF-peg11-biotin-streptavidin immobilized. The plate was incubated for two hours at room temperature on a shaking platform set at 300 rpm. Wells were washed 4× with PBST and 250 μL of a 1:1,000 dilution of an anti-Human AP secondary added to each well and incubated for 1.5 hours. Wells were washed 2× with PBST and 2× with distilled water. 250 μL of 4 mg/ml PnPP in diethanolamine was added to each well and the change in absorbance overtime at 405 nm was assessed for 10 minutes. The change in absorbance overtime for the MMAE/MMAF and trastuzumab-vc-MMAE wells were normalized to wells treated with anti-MMAE fab alone to determine the bound fraction of anti-MMAE fab.

### Surface Plasmon Resonance

A SR7500DC surface plasmon resonance was utilized for binding assessments of anti-MMAE fab. MMAF-peg11-biotin was flowed over the left channel of a neutravidin immobilized SPR chip. Subsequently unbound sites on both channels were blocked by injection of free biotin. ABC3315 fab was serially injected for 3 minutes at concentrations of 1.23, 3.70, 11.11, 33.33 and 100 nM with a 3-hour dissociation step after the final 100 nM injection. To evaluate ABC3315 binding to free MMAE and PV ABC3315 was conjugated to nhs-peg12-biotin and injected over the left channel of a streptavidin SPR chip. Subsequently, unbound sites on both channels were blocked by injection of free biotin. MMAE was serially injected for 3 minutes at concentrations of 0.37, 1.11, 3.33, 10, and 30 nM with a 3-hour dissociation step after the final 30 nM injection. A second kinetic titration was completed with PV injected at conjugated MMAE concentrations of 1.23, 3.70, 11.11, 33.33 and 100 nM with a 3-hour dissociation step after the final 100 nM injection. Observed sensorgrams were fit using the kinetic titration module of ClampXP to obtain the association rate constant, dissociation rate constant and equilibrium dissociation rate constant.

### Cell-Viability Assays

Ramos cells at a density of 50,000 cells/mL were split into individual wells of a 96-well flat-bottom culture plate. Media containing dilutions of MMAE or PV with or without 500 nM anti-MMAE fab was added to individual wells and the cells incubated for 4-days at 37° C. in a humified incubator with 5% CO2. On the fourth day 25 μL of 4 mg/ml MTT was added to individual wells and the plates incubated for 2-hours. Subsequently, MTT was solubilized overnight following the addition of 100-μL of 10% SDS, 0.1 M HCL. The absorbance of individual wells was read at a wavelength of 550 nm and 690 nm. A similar protocol was followed to evaluate the impact of anti-MMAE fab on the efficacy of trastuzumab-vc-MMAE and MMAE on SKBR3 cells. SKBR3 were trypsinised and diluted to a concentration of 40,000 cells/mL. 100 μL of the cell suspension was added to individual wells of a 96-well u-bottom culture plate and cells allowed to attach overnight. The following day the culture media was aspirated and replaced with fresh media containing MMAE or trastuzumab-vc-MMAE with and without 500 nM anti-MMAE fab. Cells were incubated for 6-days with fresh media and drug dilutions added on days 3 and 5. Cell-viability was assessed using the same MTT protocol described for the RAMOS cells. Cell viability was determined as the difference in absorbance at 550 nm and 690 nm for the treated wells divided by the difference for untreated wells. The observed cell-viability for each group was fit to a four-parameter inhibitor vs response equation in GraphPad Prism 7.

### Xenograft Efficacy Studies

Male and female Nu/J mice (The Jackson Laboratory) were injected above the right hind leg with 5×10⁶ Ramos cells in 100 μL of DPBS. Xenograft growth was monitored using digital vernier calipers and tumor volume calculated as W²×L/2 where L is the longest tumor diameter and W is the tumor diameter perpendicular to L. At a tumor volume of 200-300 mm³ (average ~250 mm³) mice were randomized in groups of 8 to receive (i) PBS+PBS control, (ii) 1 mg/kg PV+PBS, (iii) 1 mg/kg+12×ABC3315, (iv) 3 mg/kg PV+PBS (v) 3 mg/kg+12×ABC3315. PV was administered by injection into the retroorbital venous sinus. ABC3315 was administered by intraperitoneal injection and was divided into 5 injections, with 30% of the dose administered immediately after the PV injection, 25% administered 8-hours after PV, and 15% administered at 24-hours, 32-hours, and 48 hours. Tumor volumes and body weight were monitored daily, and mice were sacrificed at a tumor volume of 2000 mm³. Kaplan-Meier survival curves were generated in GraphPad Prism 7 and compared using the log-rank test at a significance level of p≤0.05.

### Toxicity Studies

The toxicity evaluation of PV following a 120 mg/kg dose with and without co-administration of 3×ABC3315 was contracted to Champions Oncology (Rockville, MD). Swiss-webster mice (n=5/group) were injected with PV by tail-vein injection. ABC3315 was administered via intraperitoneal injection following the same dosing protocol outlined in the xenograft studies methods. Control mice received an equivalent volume of PBS at each time. Mouse body weight was measured daily for fourteen days after the PV injection. Mice that lost greater than 10% of their body weight were provided with diet gel ad libitum.

### Example 2

This Example provides a description of results obtained using the materials and methods of Example 1.

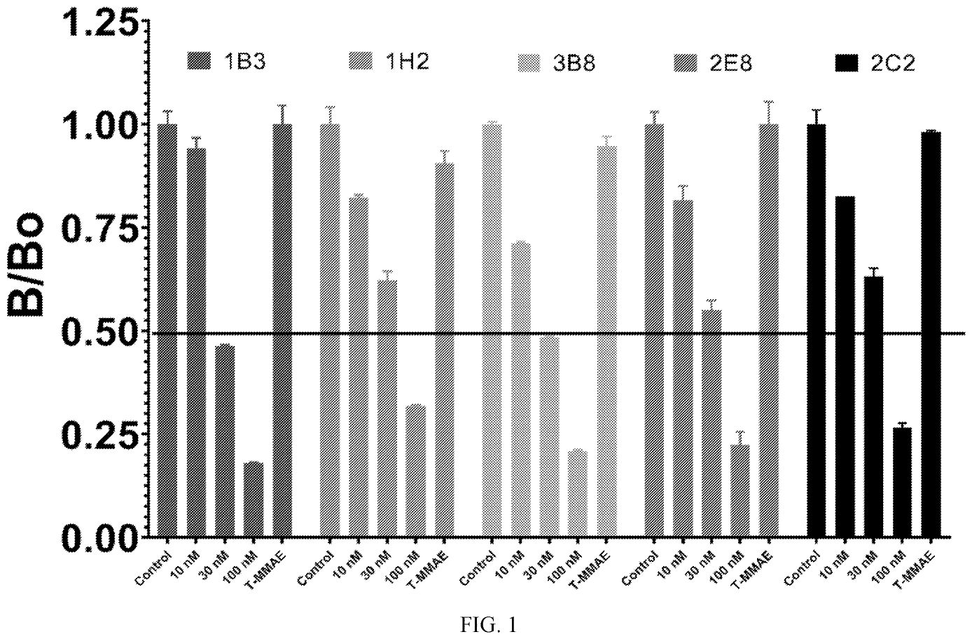

A scFv phage library was developed from spleen cells obtained from immunized mice, and the library was screened to identify scFv with selective binding for free MMAE (relative to vc-MMAE conjugates). Shown in FIG. 1 are the top 5 hits that demonstrated binding to free MMAE with negligible binding to trastuzumab-vc-MMAE (T-vc-MMAE). Bars represent the fraction of phage displaying scFv that is bound to MMAF-peg11-biotin-streptavidin with MMAE or T-vc-MMAE spiked in relative to a control well. Amino acid sequences for the clones are provided in FIG. 2.

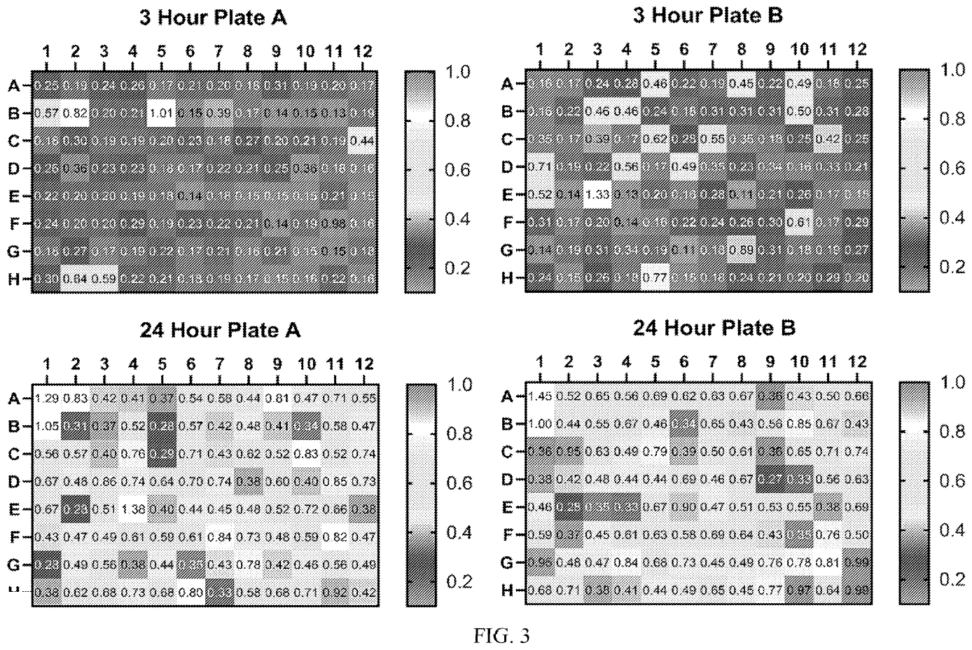

A random mutagenesis phage library was developed from clones 1H2, 1B3 and 3B8 and was subsequently panned and screened to identify clones with increased MMAE affinity. FIG. 3 shows the fractional binding signal that was observed for individual clones from 4 96-well plates with and without incubation with 10 nM or 1 nM free MMAE. The clones were sequenced, and amino acid sequences aligned (FIG. 4).

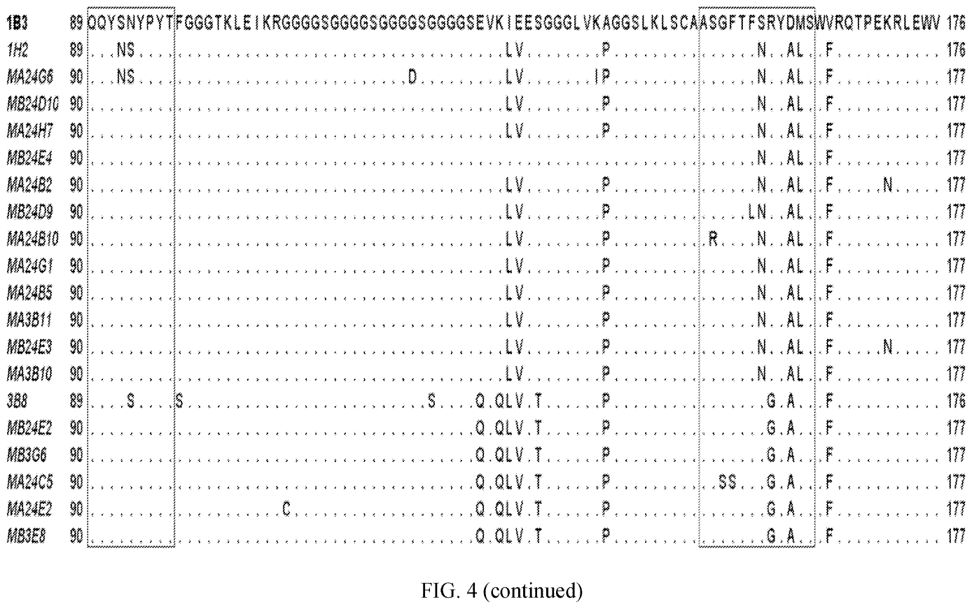

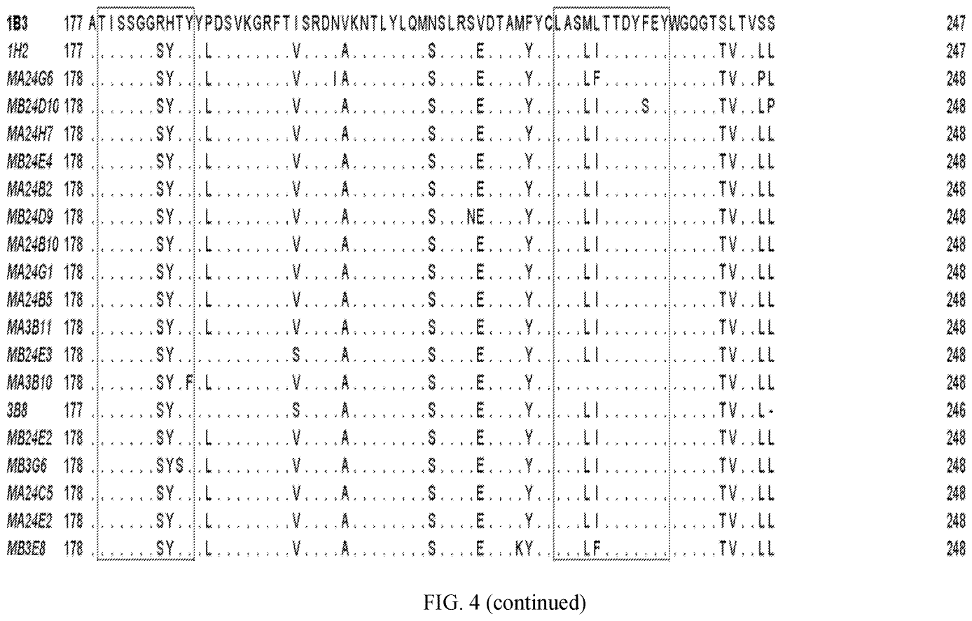

A humanized anti-MMAE fab (ABC3315) was generated by a resurfacing protocol, where the z-score of the variable domains increased following humanization from −0.624 to 0.872 for the heavy chain and from 0.120 to 0.989 for the light chain. ABC3315 was characterized using a competitive ELISA with increasing concentrations of free MMAE/MMAF and T-vc-MMAE (FIG. 5). ABC3315 binding was decreased by the addition of free MMAE and MMAF with an IC50 of ~1 nM, whereas no knockdown in binding signal was observed with the addition of T-vc-MMAE. For high affinity interactions, IC50 values observed in competitive ELISA experiments are often greater than the equilibrium dissociation constant. To better characterize the binding affinity of ABC3315, a series of kinetic titration surface plasmon resonance (SPR) analysis was performed. Rate constants were estimated for ABC3315 binding to MMAF-peg11-biotin bound to a neutravidin SPR chip. The observed sensorgram is provided in the left panel of FIG. 6. The estimated equilibrium dissociation constant for ABC3315 binding to MMAF-peg11-biotin-neutravidin was 44.2 μM. A follow up SPR experiment was completed with biotinylated ABC3315 immobilized on a streptavidin chip. Shown in the middle panel of FIG. 6 is the observed sensorgram for free MMAE injected over immobilized ABC3315. The fit equilibrium dissociation constant for free MMAE binding to ABC3315 is 7.7 μM. The higher affinity for free MMAE is the result of a faster association rate constant for ABC3315 to free MMAE (2.67×10⁵ M⁻¹sec⁻¹) in comparison to immobilized MMAF-peg11-biotin (4.11×10⁴ M⁻¹sec⁻¹). Shown in the right panel of FIG. 6 is the observed sensorgram following a kinetic titration of polatuzumab vedotin (PV) over immobilized ABC3315. Consistent with ABC3315 selectively binding free MMAE no binding signal is observed, with minimal change in the response units during the binding analysis (±2 μRU).

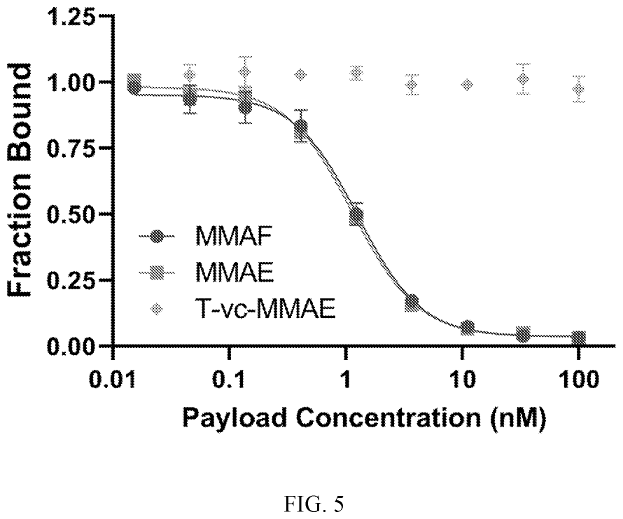

A Burkitt's lymphoma cell-line (Ramos) was incubated with free MMAE or PV with and without 500 nM ABC3315. The cell survival fraction was determined following a 4-day incubation and was calculated as the quotient of the MTT signal for treated wells divided by control wells. The addition of ABC3315 increased the observed IC50 of free MMAE in RAMOS cells from 0.12 nM to 95.96 nM. The IC50 for cells treated with PV was 0.12 nM and was 0.13 nM for PV+ABC3315. Comparable results were obtained in the HER2+ SKBR3 cell-line following treatment with free MMAE or trastuzumab-vc-MMAE. The IC50 of free MMAE was increased from 0.09 nM to 46.24 nM with ABC3315, and the IC50 of T-vc-MMAE was 0.04 nM and 0.03 nM with and without co-incubation with ABC3315. ABC3315 increased the ratio of ADC potency/payload potency by 738-fold for PV in Ramos cells and by 385-fold for T-vc-MMAE in SKBR3 cells. The observed cell-viability curves for free MMAE, PV and T-vc-MMAE are provided in FIG. 7.

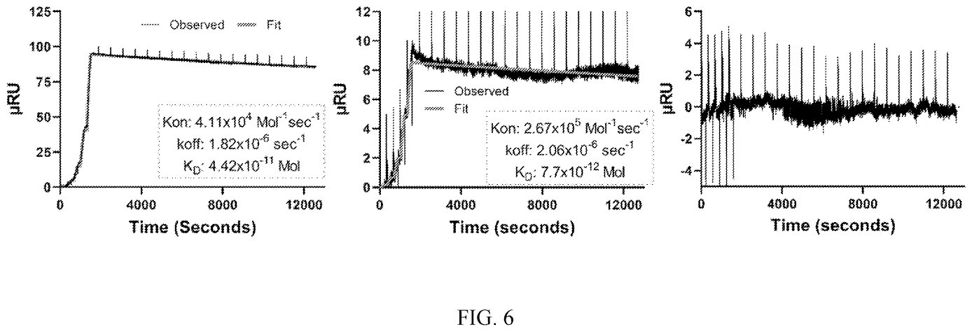

To evaluate the impact of ABC3315 on PV efficacy in vivo, NU/J mice bearing RAMOS xenografts were injected with a single dose of PV alone or with co-administration of ABC3315 at a 12-fold molar ratio (relative to conjugated MMAE). Tumor growth curves for each group are shown in FIG. 8A, and survival curves for each group are provided in FIG. 8B. Using the Log-Rank test, co-treatment of ABC3315 did not significantly alter survival for mice treated with PV at a dose of 1 mg/kg (p=0.075) or at 3 mg/kg (p=0.89).

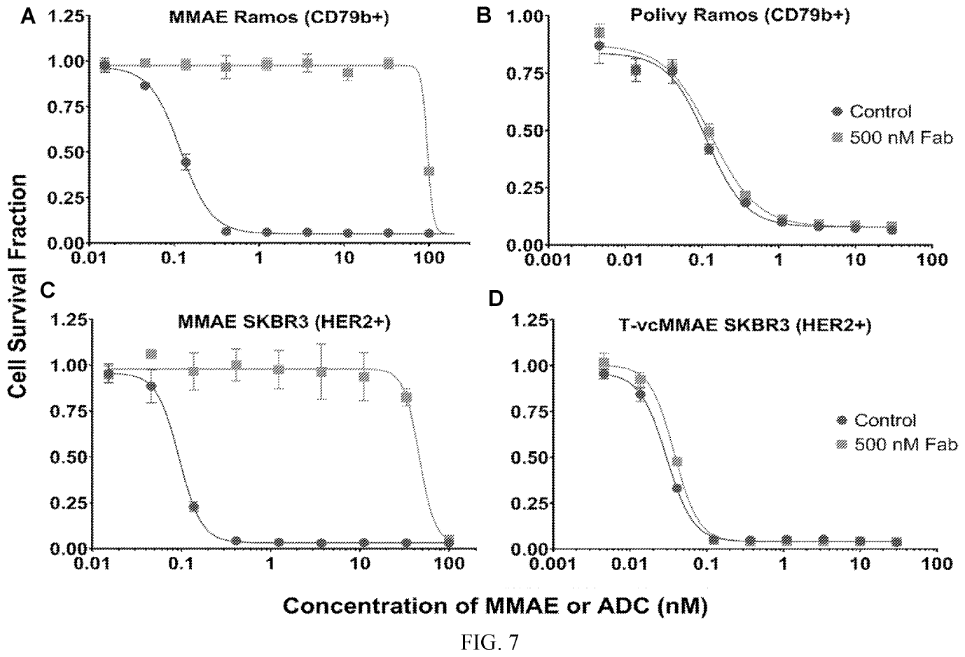

To evaluate the effect of ABC3315 on PV toxicity, Swiss-Webster mice were intravenously injected with 120 mg/kg PV with a PBS vehicle or with co-dosing of ABC3315 at a 3-fold molar ratio relative to conjugated MMAE. Mouse body weight was measured daily for 14-days after administration. The observed time-course of mean body weights for each group is provided in FIG. 9A and the nadir body weight for each group is provided in FIG. 9B. Administration of ABC3315 with 120 mg/kg PV decreased the nadir mouse body weight loss from 11.9% with a standard deviation of ±7.0% (for mice treated with the ADC with co-administration of PBS) to 4.1±2.1% (for mice treated with the ADC with co-administration of ABC3315, p=0.045).

### Example 3

### Methods

### Albumin Binding Anti-MMAE Fab

Genetic sequences for albumin binding peptides previously reported (abandoned Genentech patent application: US20050287153A1) were added to the C-terminus of the heavy chain of ABC3315. Variants were expressed in ExpiCho-S cells following manufacturer recommendations. Human serum albumin binding affinity was assessed using a surface plasmon resonance (SPR) method. For the SPR assessment human seruming (HSA) was immobilized onto a carboxymethyl dextran chip. ABC3315-WEDD was injected over the HSA chip at a concentration range from 61.2 nM to 11.4 μM for 2 minutes with a 1-minute dissociation. ABC3315-WE was injected over the HSA chip at concentrations ranging from 637 nM to 30 μM with a 2-minute infusion and a 1-minute dissociation. The observed sensorgrams were fit to obtain the association rate constant, dissociation rate constant and equilibrium dissociation rate constant for HSA binding.

### Albumin Binding Variant Sequences

Albumin binding peptides underlined. The light chain sequence of ABC3315 is unmodified for the albumin binding variants.

ABC3315-WEDD
(SEQ ID NO: 51)
EVQLVESGGGLVKPGGSLKLSCAASGFTFSGYAMSWFRQAPEKRLEW
VATISSGGSYTYYLDSVKGRFTISRDNSKNTLYLQMSSLRSEDTAVYYCLASLITTDYF
EYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CGGGSDICLPRWGCLWEDD
ABC3315-WE
(SEQ ID NO: 52)
EVQLVESGGGLVKPGGSLKLSCAASGFTFSGYAMSWFRQAPEKRLEWVA
TISSGGSYTYYLDSVKGRFTISRDNSKNTLYLQMSSLRSEDTAVYYCLASLITTDYF
EYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPK
SCGGGSDICLPRWGCLWE

### ABC3315 Mutants

Variants of ABC3315 with improved affinity or humanness have been generated. To identify ABC3315 mutants with increased affinity relative to ABC3315 a mutant scFv phage library was generated based on an ABC3315 scFv sequence using error prone PCR. Phage displaying scFv mutants were bound to MMAF-peg11-biotin-streptavidin magnetic beads for one hour and subsequently scFv displaying phage was dissociated from the beads for 24 hr, 72 hr and 120 hr for the 1^{st}, 2^{nd}, and 3^{rd} round of panning, with 1 μM of free ABC3315 included in the dissociation buffer to prevent scFv phage from rebinding during the dissociation. Following each round of dissociation, remaining scFv displaying phage was eluted using 10 mg/mL trypsin for 20 minutes. Eluted phage was amplified following each round and amplified phage was used as the input titer for the following round of panning. Following the third round of panning a small amount of the phage eluate was titered and the remaining phage amplified. From the titered phage, 2-96 well master plates were generated, and phage screened using a dissociation ELISA. Briefly, 10 μg/ml streptavidin was immobilized onto wells of a Nunc® Immobilizer™ Amino (Thermo Scientific) plate overnight. The following day, unreacted sites were blocked with 10 mM ethanolamine in pH 9.5 sodium bicarbonate buffer, subsequently, MMAF-peg11-biotin was bound to immobilized streptavidin. Amplified phage from individual wells of the 2-master plates were diluted 4× into PBS and added in duplicate to the plates and incubated for one hour on a shaking incubator. Plates were washed 3× and either PBS or PBS with 1 μM MMAF added to the wells. Plates were incubated for 24-hours, washed 3× and an anti-CM13 phage secondary added to the wells. Bound phage with 1 μM MMAF was normalized to the bound phage for each clone with PBS only treatment. Wells that were observed to have minimal change with MMAF incubation were predicted to have slow dissociation rates and were sequenced. From the phage that was amplified following the third round of panning, phagemid DNA was isolated, digested using NdeI and XhoI restriction enzymes, scFv DNA isolated using agarose gel electrophoresis, scFv DNA ligated into pet22b and transformed into SHuffle E. coli cells. The following day, individual transformed cells were inoculated into the inner wells of an ELISA plate and grown overnight. The following day 10 μL of media from each well was transferred to a 96-well deep well plate and placed in a shaker incubator for 4-hours at 30° C. 1 mM IPTG was added to each well and cells incubated overnight in the shaker incubator at 16-C. The following day cells were lysed and diluted in PBS containing HRP labeled ABC3315. The solution was transferred to MMAF-peg11-biotin neutravidin coated plates that were pre-blocked with 2% milk. The plates were incubated for 2-hours, washed and bound ABC3315-HRP evaluated following the addition of Turbo TMB substrate. Clones that decreased ABC3315-HRP signal were sequenced to identify scFv clones that efficiently competed with ABC3315 for substrate binding. To generate mutants with increased humanness, murine residues from the variable heavy chain of ABC3315 that were retained during the humanization process—due to their location in positions that may impact MMAE binding—were mutated using a Q5® site directed mutagenesis kit (New England BioLabs) to amino acid residues that commonly appear in human sequences. Mutants were expressed in ExpiCho-S cells following manufacturer recommendations. Mutants were compared to ABC3315 for binding activity using an indirect ELISA method with MMAF-peg11-biotin-neutravidin as the capture substrate and anti-human Fab alkaline phosphatase as the detection antibody.

### Results

### Human Serum Albumin Binding

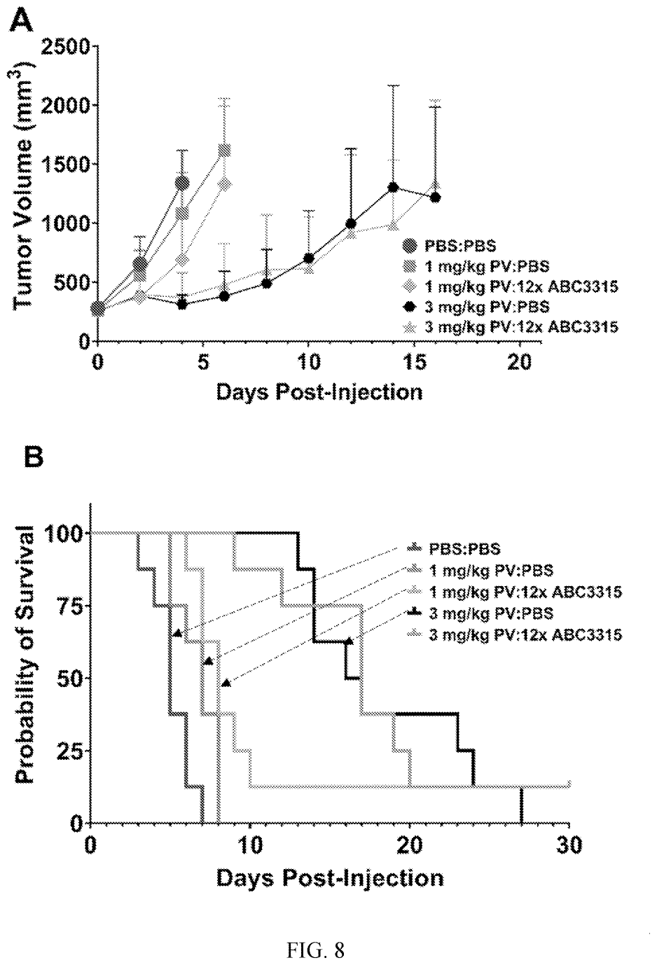

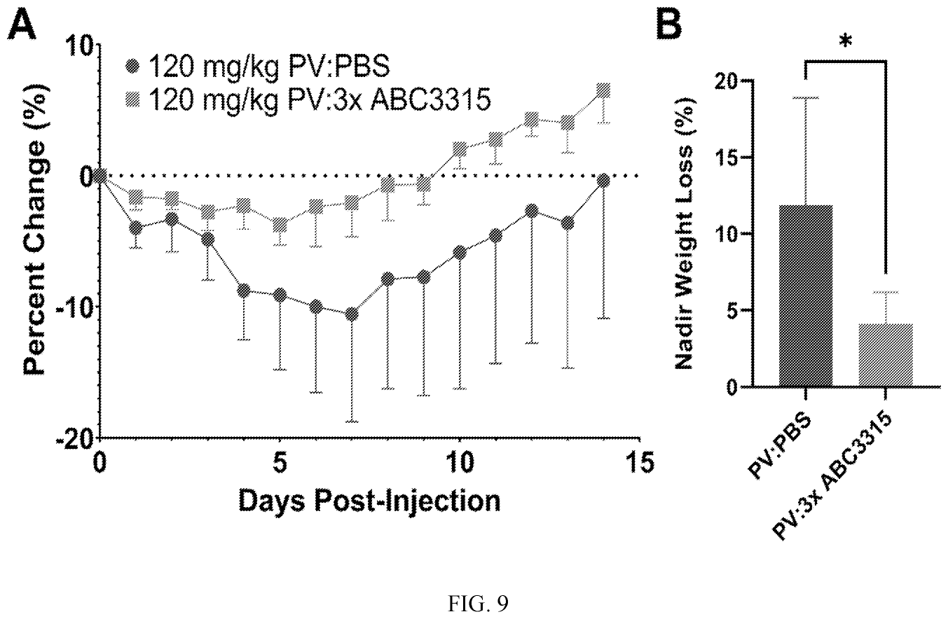

Both ABC3315-WEDD and ABC3315-WE were observed to bind immobilized human serum albumin. The observed sensorgrams for ABC3315-WEDD and ABC3315-WE are provided in FIG. 10 and FIG. 11, respectively. The best fit association rate constant, dissociation rate constant and equilibrium dissociation rate constant are provided within the insets of each figure. The estimated equilibrium dissociation rate constant for ABC3315-WEDD to human serum albumin is 2.34 μM. The estimated equilibrium dissociation rate constant for ABC3315-WE to human serum albumin is 10.50 μM. As shown in the bottom panel of both FIG. 10 and FIG. 11, similar values for the equilibrium dissociation rate constant were obtained using the observed maximum signal at each concentration.

### ABC3315 Mutants

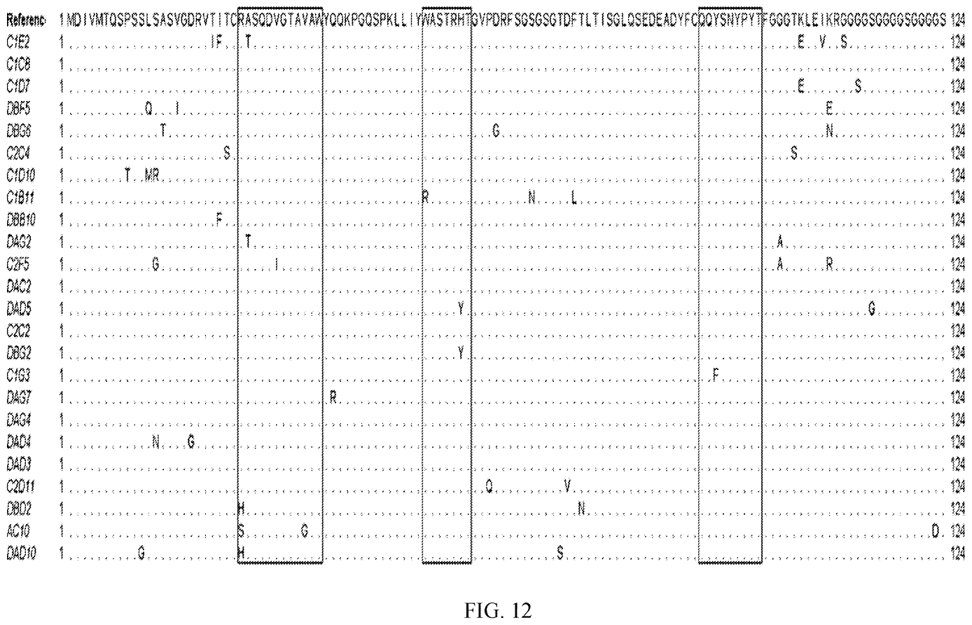

Unique ABC3315 mutants were identified following panning and screening of the mutant scFv phage display library (as shown in FIG. 12). Several clones were identified with CDR mutations that are of interest in developing an ABC3315 derivative with increased MMAE binding affinity. Four clones were identified with a phenylalanine (F) to leucine (L) mutation at position 27 of the heavy chain, therefore, this substitution was chosen for further characterization. The mutant ABC3315 Fabs with increased humanness (VH_E42G, VH_R44G, VH_L61A, VH_F37V) or potential increased MMAE affinity (VH_F27L) were compared using an indirect ELISA method. Most of the ABC3315 mutants showed a similar binding signal to immobilized MMAF-peg11-biotin as ABC3315, indicating retained binding function following mutagenesis. The VH_F37V clone showed a greatly decreased binding signal relative to ABC3315 demonstrating that the phenylalanine residue at position 37 of the heavy chain is necessary for high affinity binding. ABC3315 and VH_F27L were compared in a competitive SKBR3 cell cytotoxicity assay as described, however, the concentration of Fab was kept in a 5-fold excess relative to MMAE in-place of a constant 500 nM concentration of Fab for all MMAE dilutions. SKBR3 cells that were treated with MMAE and VH_F27L were observed to have a greater cell survival fraction relative to cells treated with MMAE and ABC3315 (FIG. 14). The observed results are consistent with VH-F27L having higher affinity for free MMAE than ABC3315.

### Mutant Heavy Chain Sequences

VH_E42G (Humanness)
(SEQ ID NO: 53)
EVQLVESGGGLVKPGGSLKLSCAASGFTFSGYAMSWFRQAPGKRLEWVATISSGGSY
TYYLDSVKGRFTISRDNSKNTLYLQMSSLRSEDTAVYYCLASLITTDYFEYWGQGTLV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSC
VH_R44G (Humanness)
(SEQ ID NO: 54)
EVQLVESGGGLVKPGGSLKLSCAASGFTFSGYAMSWFRQAPEKGLEW
VATISSGGSYTYYLDSVKGRFTISRDNSKNTLYLQMSSLRSEDTAVYYCLASLITTDYF
EYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
C
VH_L61A (Humanness)
(SEQ ID NO: 55)
EVQLVESGGGLVKPGGSLKLSCAASGFTFSGYAMSWFRQAPEKRLEWVATISSGGSY
TYYADSVKGRFTISRDNSKNTLYLQMSSLRSEDTAVYYCLASLITTDYFEYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSC
VH_F37V (Humanness)
(SEQ ID NO: 56)
EVQLVESGGGLVKPGGSLKLSCAASGFTFSGYAMSWVRQAPEKRLEW
VATISSGGSYTYYLDSVKGRFTISRDNSKNTLYLQMSSLRSEDTAVYYCLASLITTDYF
EYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
C
VH_F27L (Affinity)
(SEQ ID NO: 4)
EVQLVESGGGLVKPGGSLKLSCAASGLTFSGYAMSWFRQAPEKRLEW
VATISSGGSYTYYLDSVKGRFTISRDNSKNTLYLQMSSLRSEDTAVYYCLASLITTDYF
EYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
C
Mutant scFv sequences
>C1B11
(SEQ ID NO: 57)
MDIVMTQSPSSLSASVGDRVTITCRASQDVGTAVAWYQQKPGQSPKLL
IYRASTRHTGVPDRFSGNGSGTDLTLTISGLQSEDEADYFCQQYSNYPYTFGGGTKLE
IKRGGGGSGGGGSGGGGSGGGGSEVQLVESGGGLVKPGGSLKLSCAASGFSFSGYA
MSWFRQAPDKRLEWVATISSGGSYTYYLDSVKGRFTISRDNSKNTLYLQMSSLRSED
TAVYYCLASLITTDYFEYWGQGTLVTVSS
>C1C8
(SEQ ID NO: 58)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRPEWVATISSGGSYTYYLDSVKGRFTISRDNNKNTLFLQMSSLRSED
TAVYYCLASMITTDYFEYWGQGTLVTVSS
>C1D7
(SEQ ID NO: 59)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTELEI
KRGGSGSGGGGSGGGGSGGGGSEVQLVESGGGLVKPGGSLKLSCAASGFTFSGYAM
SWFRQAPEKRLEWVATISSGGSYTYYLDSVKGRFTISRDNSKNTLYLQMSSLRSEDTA
VYYCLASLVTTDYFEYWGQGTLVTVSS
>C1D10
(SEQ ID NO: 60)
MDIVMTQSTSSMRASVGDRVTITCRASQDVGTAVAWYQQKPGQSPKL
LIYWASTRHTGVPDRFSGSGSGTDFTLTISGLQSEDEADYFCQQYSNYPYTFGGGTKL
EIKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>C1E2
(SEQ ID NO: 61)
MDIVMTQSPSSLSASVGDRVIFTCRTSQDVGTAVAWYQQKPGQSPKLLI
YWASTRHTGVPDRFSGSGSGTDFTLTISGLQSEDEADYFCQQYSNYPYTFGGGTELE
VKRSGGGSGGGGSGGGGSGGGGSEVQLVESGGGLVKPGGSLKLSCAASGFTFSGYA
MSWFRQAPEKRLEWVATISSSGSYTYYLDSVKGRFTISRDNSKNTLYLQMSSLRSEDT
AVYYCLASLMTTDYFEYWGQGTLVTVSS
>C1G3
(SEQ ID NO: 62)
MDIVMTQSPSSLSASVGDRVTITCRASQDVGTAVAWYQQKPGQSPKLL
IYWASTRHTGVPDRFSGSGSGTDFTLTISGLQSEDEADYFCQQFSNYPYTFGGGTKLEI
KRGGGGSGGGGSGGGGSGGGGSEVQLVESGGGLVKPGGSLKLSCAASGLTFSGYAM
SWFRQAPEKRLEWVATISSGGSYTYYLDSVKGRFTISRDNSKKTLYLQMSSLRSEDTA
VYYCLASLITTDYFEYWGQGTLVTVSS
>C2C2
(SEQ ID NO: 63)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSGVQLVESGGGLVKPGGSLKLSCAASGFTISGYA
MSWFRQTPEKRLEWVATISSGGSYTYYLDSVKGRFTTSRDNSINTLYLQMSSLRSEDT
AVYYCLASLFTTDYFEYWGQGTLVTVNS
>C2C4
(SEQ ID NO: 64)
MDIVMTQSPSSLSASVGDRVTISCRASQDVGTAVAWYQQKPGQSPKLLIYWASTRHT
GVPDRFSGSGSGTDFTLTISGLQSEDEADYFCQQYSNYPYTFGGGSKLEIKRGGGGSG
GGGSGGGGSGGGGSEVQLVESGGGLVKPGGSLKLSCAASGFTFSGYAMSWFRQTPE
KRLEWVATISSGGSYTYYLDSVKGRFTISRDNSKNTLYLQMSSLRSEDTAVYYCLASL
ITTDYFEYWGQGTLVTVSS
>C2D11
(SEQ ID NO: 65)
MDIVMTQSPSSLSASVGDRVTITCRASQDVGTAVAWYQQKPGQSPKLL
IYWASTRHTGVQDRFSGSGSGTVFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPGKRLEWVATISSGGSYTYYLDSVKGRFTISRDNSKNTLYLQMSSLRSED
TAVYYCLASLITTDYFEYWGQGTLVTVSS
>C2F5
(SEQ ID NO: 66)
MDIVMTQSPSSLGASVGDRVTITCRASQDIGTAVAWYQQKPGQSPKLLI
YWASTRHTGVPDRFSGSGSGTDFTLTISGLQSEDEADYFCQQYSNYPYTFGAGTKLEI
RRGGGGSGGGGSGGGGSGGGGSEVQLDESGGGLVKPGGSLKLSCAASGFTFSGYAM
SWFRQAPDKRLEWVATISSGGSYTYYLDSVKGRFTISRDNSKNTLYLQMSSLRSEDTA
VYYCLASLITTDYFEYWGQGTLVTVSS
>DBG6
(SEQ ID NO: 67)
MDIVMTQSPSSLSTSVGDRVTITCRASQDVGTAVAWYQQKPGQSPKLLI
YWASTRHTGVPGRFSGSGSGTDFTLTISGLQSEDEADYFCQQYSNYPYTFGGGTKLEI
NRGGGGSGGGGSGGGGSGGGGSEVQLVESGGGLVKPGGSLKLSCAASGFTFSGYAM
SWFRQAPEKRLEWVATISSGGSYTYYLDSVKGRFTVSRDNSKNTLYLQMSSLRSEDT
AVYYCLASLITTDYFEYWGQGTLVTVSS
>DAD3
(SEQ ID NO: 68)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVERGGSLKLSCAASGFTFSGYA
MSWFRQAPEKRLEWVATISSGGSYTYYLDSVKGRFTISRDNSKNTLYLQMSSLRSED
TAVYYCLASLLTTDYFEYWGQGTLVTVSS
>DAD4
(SEQ ID NO: 69)
MDIVMTQSPSSLNASVGGRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGLTFSGYA
MSWFRQAPEKRLEWVGTISSGGSYTYYLDSVKGRFTISRDNSKNTLYLQMSSLRSED
TAVYYCLASLITTDYFEYWGQGTLVTVSS
>DAG4
(SEQ ID NO: 70)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMSSLRSED
TAVYYCLASLITTDYLEYWGQGTLVTVSS
>DAC2
(SEQ ID NO: 71)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGSYTHYLDSVKGRFTISRDNSKNTLYLQMSSLRSED
TAVYYCLASLITTDYFEYWGQGTLVTVSS
>DBF5
(SEQ ID NO: 72)
MDIVMTQSPSSQSASIGDRVTITCRASQDVGTAVAWYQQKPGQSPKLLI
YWASTRHTGVPDRFSGSGSGTDFTLTISGLQSEDEADYFCQQYSNYPYTFGGGTKLEI
ERGGGGSGGGGSGGGGSGGGGSEVQLVESGGGLVKPGGSLKLSCAASGFTFSGYAM
SWFRQAPEKRLEWVATISSGGSYTYYLDSVKGRFTISRDNSKNTLYLQTSSLRSEDTA
VYYCLASLITTDYFEYWGQGTLVTVSS
>DAD10
(SEQ ID NO: 73)
MDIVMTQSPSGLSASVGDRVTITCHASQDVGTAVAWYQQKPGQSPKLL
IYWASTRHTGVPDRFSGSGSGSDFTLTISGLQSEDEADYFCQQYSNYPYTFGGGTKLE
IKRGGGGSGGGGSGGGGSGGGGSEVQLFESGGGLVKPGGSLKLSCAASGFTFSGYA
MSWFRQAPEKRPEWVATISSGGSYTYYLDSVKGRFTISRDNSKNTLYLQMSSLRSED
TAVYYCLASLITTDYFEYWGQGTLVTVSS
>AC10
(SEQ ID NO: 74)
MDIVMTQSPSSLSASVGDRVTITCSASQDVGTAGAWYQQKPGQSPKLL
IYWASTRHTGVPDRFSGSGSGTDFTLTISGLQSEDEADYFCQQYSNYPYTFGGGTKLE
IKRGGGGSGGGGSGGGDSGSGGSEVQLVESGGGLVKRGGSLKLSCAASGFTLSGYA
MSWFRQAPGKRPEWVATISSGGSYTYYLDSVKGRFTISRDNSKNTLYLQMSSLRSED
TAVYYCLASLITTDYFEYWGQGTLVTVSS
>DBB10
(SEQ ID NO: 75)
MDIVMTQSPSSLSASVGDRVTFTCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DBD2
(SEQ ID NO: 76)
MDIVMTQSPSSLSASVGDRVTITCHASQDVGTAVAWYQQKPGQSPKLL
IYWASTRHTGVPDRFSGSGSGTDFNLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPDKRLEWVATISSGGGYTYYLDSVKGRFTISRDNSKNTLYLQMSSLRSED
TAVYYCLASLITTDYFEYWGQGALVTVSS
>DBG2
(SEQ ID NO: 77)
MDIVMTQSPSSLSASVGDRVTITCRASQDVGTAVAWYQQKPGQSPKLL
IYWASTRYTGVPDRFSGSGSGTDFTLTISGLQSEDEADYFCQQYSNYPYTFGGGTKLE
IKRGGGGSGGGGSGGGGSGGGGSKVQLVESGGGLVKRGGSLKLSCAASGFTFSGYA
MSWFRQAPEKRLEWVATISSGGSYTYYLDSVKGRFTISRDNSKNTLYLQLSSLRSEDT
AVYYCLASLITTDYFEYWGQGTLVTVSS
>DAD5
(SEQ ID NO: 78)
MDIVMTQSPSSLSASVGDRVTITCRASQDVGTAVAWYQQKPGQSPKLL
IYWASTRYTGVPDRFSGSGSGTDFTLTISGLQSEDEADYFCQQYSNYPYTFGGGTKLE
IKRGGGGGGGGGSGGGGSGGGGSEVQLVESGGGLVKPGGSLKLSCAASGLTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DAG2
(SEQ ID NO: 79)
MDIVMTQSPSSLSASVGDRVTITCRTSQDVGTAVAWYQQKPGQSPKLLI
YWASTRHTGVPDRFSGSGSGTDFTLTISGLQSEDEADYFCQQYSNYPYTFGAGTKLEI
KRGGGGSGGGGSGGGGSGGGGSEVQLVESGGGLVKPGGSLKLSCAASGLTFSGYAM
SWFRQAPEKRLEWVATISSGGSYTYYLDSVKGRFTISRDNSKNTLYLQMSSLRSEDTA
VYYCLASLITTDYFEYWGQGTLVTVSS
>DAG7
(SEQ ID NO: 80)
MDIVMTQSPSSLSASVGDRVTITCRASQDVGTAVAWYROKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKGGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS

### Example 4

This Example includes sequences of additional described antibodies, and characterization of antibody referred to herein as ABC3315.

>DAB6
(SEQ ID NO: 81)
MDIVMTQSPSSLSASVGDRVTIACRASQDVGTAVAWYQQKPGQSPKLL
IYWASTRHTGVPDRFSGSGSGTDFTLTISGLQSEDEADYFCQQYSNYPYTFGGGTKLE
VKRGGGGSGGGGSGSGGSGGGGSEVQLVESGGGLVKPGGSLKLSCAASGSTFSGYA
MSWFRQAPDKRLEWVATISSGGSYTYYLDSVKGRYTISRDNSKNTLYLQMSSLRSED
TAVYYCLASRITTDYFEYWGQGTLVTVSS
>DAC3
(SEQ ID NO: 82)
MDIVMTQSPSSLSASVGDRVTITCRASQDVGAAVAWYKKKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DAC4
(SEQ ID NO: 83)
MDIVMTQSPSSLSASVGDRVTITCHASQDVGTAVAWYQQKPGQSPKLL
IYWASTRHTGVPDRFSGSGSGTDFTLTISGLQSEDEADYSCQQYSNYPYTFGGGTKLE
IKRGGGGSGGGGSGGGGSGGGGSEVQLVESGGGRVKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DAC9
(SEQ ID NO: 84)
MDIVMTQSPSSLSASVGDRVTITCHASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPDKRLEWVATISSGGSYTYYLDSVKGRFTISRDNSKNTLYLQMSSLRSED
TAVYYCLASLITTDYFEYWGQGTLVTVSS
>DAD9
(SEQ ID NO: 85)
MDIVMTQSPSSLSASVGDRVTITCRASQDVGTAVAWYQQKPGQSPKLL
IYWASTRHTGVPGRFSGSGSGTDFTLTIRGLQSEDEADYFCQQYSNYPYTFGGGTKLD
IKRGGGGSGGGGSGGGGSGGGGSEVQLVESGGGLVKPGGSLKLSCAASGFTFSGYA
MSWFRQAPDKRLEWVATISSGGSYTYYLDSVKGRFTISRDNSKNTLYLQMSSLRSED
TAVYYCLASLFTTDYFEYWGQGTLVTVSS
>DAE2
(SEQ ID NO: 86)
MDIVMTQSPSSLSASVGDRVTITCRASQDVGTAVAWYQQKPGQSPKLL
IHWASTRHTGVPDRFSGSGSGTDFTLTISGLQSEDEADYFCQQYSNYPYTFGGGTKM
EIKRGGGGSGGGGSGGGGSGGGGSEVQLVESGGGLVKPGGSLKLSCAASGFTFSGYA
MSWFRQAPGKRLEWVATISSGGSYTYYLDSVKGRFTISRDNSKNTLYLQMSSLRSED
TAVYYCLASLITTDYFEYWGQGTLVTVSS
>DAG5
(SEQ ID NO: 87)
MDIVMTQSPSSLSASVGGRVTITCRASQDVGTAVAWYQQKPGQSPKLL
IYWASTRYTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGLTFSGYA
MSWFRQAPEKRLEWVATISSGGSYTYYLDSVKGRLTISRDNSKNTLYLQMSSLRSED
TAVYYCLASLITTDYFEYWGQGTLVTVSS
>DAG10
(SEQ ID NO: 88)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DBC8
(SEQ ID NO: 89)
MDIVMTQSPSSLSASVGDRVTITCRASQDVGTAVAWYQQKPGQSPKLL
IYWASTRHTGVPDRFSGSGSGADFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMNSLRSED
TAVYYCLASLITTDYFEYWGQGTLVTVSS
>DBD5
(SEQ ID NO: 90)
MDIVMTQSPGSLSASVGDRVTITCRASQDVGTAVAWYQQKPGQSPKLL
IYWASTRHTGVPDRLSGSGSGTDFTLTISGLQSEDEADYFCQQYSNYPYTFGGGTKLE
IKRGGGGSGGGGSGGGGSGGGGSEVQLVESGGGLVKPGGSLKLSCAASGSTFSGYA
MSWFRQAPEKRPEWVATISSGGSYTYYLDSVKGRFTISRDNSKNTLYLQMSSLRSED
TAVYYCLASLITTDYFEYWGQGTLVTVSS
>DBD9
(SEQ ID NO: 91)
MDIVMTQSPSSLRASVGGRVTITCRASQDVGTAVAWYQQKPGQSPKLL
IHWASTRHTGVPDRFSGSGSGTDFTLTISGLQSEDEADYFCQQYSNYPYTFGGSTKLEI
KRGGGGSGGGGSGGGGSGGGGSEVQLVESGGGLVKPGGSLKLSCAASGFTFSGYAM
SWFRQAPEKRLEWVATISSGGSYTYYLDSVKGRFTISRDNSINTLYLQMSSLRSEDTA
VYYCLASQFTTDYFEYWGQGNLVTVSS
>DBD11
(SEQ ID NO: 92)
MDNVMTQSPSSLSASVGDRVTITCHASQDVGTAVAWYQQKPGRSPKL
LIYWASTRHTGVPDRFSGSGSGTDFTLTISGLQSVDGADYFCQQYSNYPYTFGGGTKL
EIKRGGGGSGGGGSGGGGSGGGGSEVQLVESGGGLVKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DBG3
(SEQ ID NO: 93)
MDIVMTQSPSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
INRGGGGSGGGGSGGGGSGGGGSEVQLVESGGGLVKPGGSLKLSCAASGFTLSGYA
MSWFRQAREKRLEWVATISSGGSYTYYLDSVKGRFTISRDNSKNTLYLQMSSLRSED
TAVYYCLASLITTDYFEYWGQGTPVTVSS
>DBG4
(SEQ ID NO: 94)
MDIVMTQSPSSLSASVGDRVTITCRASQDVGTAVAWYQQKPGQSPKLL
TYWASTRHTGVPDRFSGSGSGTDFTLTISGLQSEDEADYSCQQYSNYPYTFGGGTKL
EIKRGGGGSGGGGSDGGGTGGGGSEVQLVESGGGLIKPGGSLKLSCAASGFTFSGYA
MSWFRQAPEKRLEWVATISSGGSYTYYLDSVKGRFTISRDNSKNTLYLQMSSLRSED
TAVYYCLASLITTDYFEYWGQGTLVTVSS
>DBG8
(SEQ ID NO: 95)
MDIVMTQSLSSLSASVGDRVTITCRASQDVGTAVAWYQQKPGQSPKLL
IYWASTRHTGVPDRFSGSGSGTDFTLTISGLQSEDEADYFCQQYSNYPYTFGGGTKLE
IKRGGGGSGGGGSGGGGSGGGGSEVQLVESGGGLVKPGGSLKLSCAASGFTFSGYA
MSWFRQAPEKRLEWVATISSGGNYTYYLDSVKGRFTISRDNSKNILYLQMSSLRSEDT
AVYYCLASLITTDYFEYWGQGTLVTVSS
>DBG11
(SEQ ID NO: 96)
MDIVMTQSPSSLSASVGDRVTITCRASQDVGTAVAWYQQNPGQSPKLL
IYWASTRHTGVPDRFSGSGSGTDFTLTISSLQSEDEADYFCQQYSNYPYTFGGGTKLEI
KRGGGGSGGGGSGGGGSGGGGSEVQLVESGGGLVKPGGSLKLSCAASGFTFSGYTM
SWFRQAPEKRLEWVATISSGGSYTYYLDSVKGRFTISRDNSKKTLYLQMSSLRSEDTA
VYYCLASLITTDYFEYWGQGTLVTVSS
ABC3317 Sequence
Light chain
(SEQ ID NO: 1)
DIVMTQSPSSLSASVGDRVTITCRASQDVGTAVAWYQQKPGQSPKLLIY
WASTRHTGVPDRFSGSGSGTDFTLTISGLQSEDEADYFCQQYSNYPYTFGGGTKLEIK
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Heavy chain (albumin binding sequence in italics)
(SEQ ID NO: 98)
EVQLVESGGGLVKPGGSLKLSCAASGFTFSGYAMSWFRQAPEKRLEW
VATISSGGSYTYYLDSVKGRFTISRDNSKNTLYLQMSSLRSEDTAVYYCLASLITTDYF
EYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CGGGSRLIEDICLPRWGCLWEDD
ABC3320 Sequence
Light chain (H55Y Mutation in bold)
(SEQ ID NO: 3)
DIVMTQSPSSLSASVGDRVTITCRASQDVGTAVAWYQQKPGQSPKLLIYWASTRYTG
VPDRFSGSGSGTDFTLTISGLQSEDEADYFCQQYSNYPYTFGGGTKLEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Heavy chain (F27L mutation in bold, albumin
binding sequence in italics)
(SEQ ID NO: 5)
EVQLVESGGGLVKPGGSLKLSCAASGLTFSGYAMSWFRQAPEKRLEWVATISSGGSY
TYYLDSVKGRFTISRDNSKNTLYLQMSSLRSEDTAVYYCLASLITTDYFEYWGQGTLV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCGGGSRLIEDI
CLPRWGCLWEDD

Newly identified sequences from Naïve Human Fab Library

Human Fab (VL and VH listed)

>hFM1
(SEQ ID NO: 177)
QAVLTQPSSVSGAPGQRVTISCTGSSSNIGAYYVHWYQQVPGTAPRLLI
FDNDNRPSGVPDRFSASKSGTSASLAIIGLQAEDEAEYYCQSVDYSLGDGVVFGGGT
KLTVL
(SEQ ID NO: 100)
QVQLVESGPGLVKPSETLSLTCAVSGGSISSNNWWSWVRQTPGKGLEW
IGEIYHSGSTNYNPSLKSRVTISVDKSKNQFSLKLSSVTAADTAVYYCANLRGQWGQG
TLVTVSS
>hFM2
(SEQ ID NO: 101)
QSVVTQPPSVSGAPGQRVTISCTGSSSNIGAGYDVHWYQQLPGTAPRL
LISGNTNRPSGVPDRFSGSKSGTSASLAITGLQAEDEADYYCQSYDSSLSGHVVFGGG
TKLTVL
(SEQ ID NO: 102)
QVQLQQWGAGLLKPSETLSLTCAVYGGSFSGYYWSWIRQPPGKGLEW
IGEINHSGSTNYNPSLKSRVTISVDTSKNQFSLKLSSVTAADTAVYYCARGKRGNFDY
WGQGTLVTVSS
>hFM3
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM4
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM5
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM6
(SEQ ID NO: 129)
QAVLTQPSSVSGAPGQRVTISCTGSSSNIGAGYDVHWYQQLPGTAPKLLIYSNTNRPS
GVPDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEW
IGEISHSGIPNYNPSLASRVTISIDRSQNQFSLKLTSVTAADTAVYYCARGGGRFDFWG
QGTLVTVSS
>hFM7
(SEQ ID NO: 137)
QAVLTQPSSVSGAPGQRVTISCTGTNSNIGAGYDVHWYQQFPGAAPKV
LIYGNTNRPSGVPGRFSASKSGTSASLAITGLQAEDEADYYCQTYDSSLSGTWVFGG
GTKLTVL
(SEQ ID NO: 138)
QVQLQQWGPGLVKPSGTLSLTCAVSGVSISSSNWWSWVRQTPGKGLE
WIGEISHSGSPNYNPSLKSRVTISVDTSKNQFSLKLTSVTAADSAVYYCARGGGPIPHI
WGQGTTVTVSS
>hFM8
(SEQ ID NO: 139)
QAVLTQPSSVSGAPGQRVTISCTGSSTNIGADFDVHWYQQLPGTAPKLL
IHGDNNRPSGVPDRFSGSKSGTSAYLAISGLQAEDEADYYCQTYDSRSSGSRVFGGGT
KVTVL
(SEQ ID NO: 107)
QLQLQESGPGLVKPSGTLSLTCAVSGVSISSRNWWSWVRQTPGKGLE
WIGEISHSGSTNYNPSLKSRVTISVDTSKNQFSLKLSSVTAADTAVYYCARNAGDIWG
QGTMVTVSS
>hFM9
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM11
(SEQ ID NO: 131)
QSVVTQPPSVSGAPGQRVTISCTGSSSNIGANYDVHWYQQLPGTAPKL
LIYGNSNRPSGVPDRFSGSKSGTSASLAITGLQAEDEADYYCQSYDSSLSGSSVFGGG
TKLTVL
(SEQ ID NO: 132)
QVQLQQSGPGLVKPSGTLSLTCDVSGGSISSRNWWTWVRQPPGKGLE
WIGEIYHSGSTNYNPSLESRVAMSVDKSRYQFSLRLSSVTAADTAVYYCARRRDGYF
DYWGQGTLVTVSS
>hFM12
(SEQ ID NO: 156)
QAVLTQPSSVSGAPGQRVAISCTGSSSNIAAGYDVQWYQQLPGTAPKLLIYGNSNRPS
GVPDRFSGSKSGTSASLAITGLQAEDEADYYCQSYDSSLSVLYVFGTGTKVTVL
(SEQ ID NO: 157)
QLQLQESGPGLVKPSGTLSLTCAVSGGSISSSNWWSWVRQPPGKGLEW
IGEIYHSGSTNYNPSLKSRVTISVDKSKNQFSLKLSSVTAADTAVYYCAKRPRNSGYL
GAFDIWGQGTMVTVSS
>hFM13
(SEQ ID NO: 158)
DVVMTQSPLSLAVTLGQPASISCRSSQSLLHSSGYKFLNWYLQKPGQSPQLLIYLGSN
RASGVPDRFSGSGSGTDFTLKISRVEAEDVGVYYCMQALQSPTFGGGTKVEIK
(SEQ ID NO: 159)
EVQLVESGGGLVQPGGSLRLSCTASGFTFSSFSMNWVRQAPGKGPEW
VSAISGSGGGTYYADSVKGRFTISRDNAKNSLYLQMSSLRAEDTAVYYCARVRVPQA
FDIWGQGTMVTVSS
>hFM14
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM15
(SEQ ID NO: 139)
QAVLTQPSSVSGAPGQRVTISCTGSSTNIGADFDVHWYQQLPGTAPKLL
IHGDNNRPSGVPDRFSGSKSGTSAYLAISGLQAEDEADYYCQTYDSRSSGSRVFGGGT
KVTVL
(SEQ ID NO: 107)
QLQLQESGPGLVKPSGTLSLTCAVSGVSISSRNWWSWVRQTPGKGLE
WIGEISHSGSTNYNPSLKSRVTISVDTSKNQFSLKLSSVTAADTAVYYCARNAGDIWG
QGTMVTVSS
>hFM16
(SEQ ID NO: 177)
QSVVTQPPSVSGAPGQRVTISCTGSSSNIGAGYDVHWYQQLPGTAPKL
LIYGDTNRPSGVPDRFSGSKSGTSASLAITGLQAEDEADYYCQSYDSSLSGSVVFGGG
TKLTVL
(SEQ ID NO: 178)
QVQLQESGPGLVKPSGTLSLTCAVSGVSISTTNWWSWVRQPPGKGLE
WIGEISHSGITNYNPSLQSRVTISVDTSKNQFSLKLNSVTAADTAVYYCARVGEGAFK
DLGQGTLVTVSS
>hFM18
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNFD
YWGQGTLVTVSS
>hFM19
(SEQ ID NO: 117)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKLTVL
(SEQ ID NO: 118)
QVQLVESGPGLVKPSGTLSLTCAVSGGSISSSDWWSWVRQPPGKGLEW
IGEMRLSGTTNYNPSLKSRVAISMDKSKNHFSLNLSSVTAADTAVYYCANADYYTHH
YWGQGTLVTVSS
>hFM20
(SEQ ID NO: 119)
QAVLTQPSSVSGAPGQRVTISCTGSSSNIGAGYDVHWYQQLPGRAPKL
LIYGNNQRPSGVPDRFSGSTSGTSASLAITGPQAEDEADYYCQSYDSSLNGIWVFGGG
TKLTVL
(SEQ ID NO: 120)
QVQLQESGPGLVKPSGTLSLTCAVSGVSISTTNWWSWVRQPPGKGLE
WIGEINPSGSTNYNPSLKSRVTMSLDTSKNQFSLKLRSVTAADTALYYCATRDYWGQ
GTLVTVSS
>hFM21
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNFD
YWGQGTLVTVSS
>hFM23
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNED
YWGQGTLVTVSS
>hFM24
(SEQ ID NO: 131)
QSVVTQPPSVSGAPGQRVTISCTGSSSNIGANYDVHWYQQLPGTAPKL
LIYGNSNRPSGVPDRFSGSKSGTSASLAITGLQAEDEADYYCQSYDSSLSGSSVFGGG
TKLTVL
(SEQ ID NO: 132)
QVQLQQSGPGLVKPSGTLSLTCDVSGGSISSRNWWTWVRQPPGKGLE
WIGEIYHSGSTNYNPSLESRVAMSVDKSRYQFSLRLSSVTAADTAVYYCARRRDGYF
DYWGQGTLVTVSS
>hFM28
(SEQ ID NO: 108)
QSVLTQPPSVSAAPGQKVTISCSGSSSDIGNNFVSWYQQLPGTAPKRLI
YDNSKRPSGIPERFSGSKSGTSATLGITGLQTGDEADYYCGAWDTSLSAYVFGTGTKV
TVL
QVQLQQWGPGLVKPSETLSLTCAVYGGSFSGYYWSWIRQPPGKGLEW
IGEINHSGSTNYNPSLKSRVTISVDTSKNQFSLKLSSVTAADTAVYYCARYSNYRHYY
YGMDVWGQGTLVTVSS
>hFM29
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM30
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNED
YWGQGTLVTVSS
>hFM31
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM32
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHNWWSWVRQTPGKGLE
WIGEISHSGITNYNPSLKSRVTISVDTSKNQLSLKLSSVTAADTAVYYCARGSTGAFDV
WGQGTMVTVSS
>hFM33
(SEQ ID NO: 169)
QSVLTQPPSVSAAPGQKVTISCSGSSSNIGNYHVSWYQQLPGTAPKLLI
YDNNKRPSGIPDRFSGSKSGTSATLDITGLQTGDEADYYCGTWDSSLSAVLFGRGTKL
TVL
(SEQ ID NO: 170)
QLQLQESGPGLVKPSGTLSLTCAVSGGSISSGYYWSWIRQPPGKGLEWI
GEINHSGSTNYNPSLKSRVTISVDTSKNQFSLKLSSVTAADTAVYYCARLRGSGRWGQ
GTLVTVSS
>hFM34
(SEQ ID NO: 169)
QSVLTQPPSVSAAPGQKVTISCSGSSSNIGNYHVSWYQQLPGTAPKLLI
YDNNKRPSGIPDRFSGSKSGTSATLDITGLQTGDEADYYCGTWDSSLSAVLFGRGTKL
TVL
(SEQ ID NO: 170)
QLQLQESGPGLVKPSGTLSLTCAVSGGSISSGYYWSWIRQPPGKGLEWI
GEINHSGSTNYNPSLKSRVTISVDTSKNQFSLKLSSVTAADTAVYYCARLRGSGRWGQ
GTLVTVSS
>hFM36
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNFD
YWGQGTLVTVSS
>hFM39
(SEQ ID NO: 145)
QAVLTQPSSVSGAPGQRVTISCTGSSSNIGAGYDVHWYQQLPGTAPKL
LIYGNSNRPSGVPDRFSGSKSGTSASLAITGLQAEDEADYYCQSYDSSLSGLYVFGTG
TKVTVL
(SEQ ID NO: 146)
QVQLQESGPGLVKPSETLSLTCAVSGVSISTTNWWSWVRQPPGKGLEW
IGEISHSGITNYNPSLKSRVTISVDKAKNQFSLMLNSVTAADTAVYYCARGYGMDVW
GQGTMVTVSS
>hFM40
(SEQ ID NO: 117)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKLTVL
(SEQ ID NO: 118)
QVQLVESGPGLVKPSGTLSLTCAVSGGSISSSDWWSWVRQPPGKGLEW
IGEMRLSGTTNYNPSLKSRVAISMDKSKNHFSLNLSSVTAADTAVYYCANADYYTHH
YWGQGTLVTVSS
>hFM41
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHINWWSWVRQTPGKGLE
WIGEISHSGITNYNPSLKSRVTISVDTSKNQLSLKLSSVTAADTAVYYCARGSTGAFDV
WGQGTMVTVSS
>hFM42
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHNWWSWVRQTPGKGLE
WIGEISHSGITNYNPSLKSRVTISVDTSKNQLSLKLSSVTAADTAVYYCARGSTGAFDV
WGQGTMVTVSS
>hFM43
(SEQ ID NO: 117)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKLTVL
(SEQ ID NO: 118)
QVQLVESGPGLVKPSGTLSLTCAVSGGSISSSDWWSWVRQPPGKGLEW
IGEMRLSGTTNYNPSLKSRVAISMDKSKNHFSLNLSSVTAADTAVYYCANADYYTHH
YWGQGTLVTVSS
>hFM44
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNED
YWGQGTLVTVSS
>hFM45
(SEQ ID NO: 99)
QAVLTQPSSVSGAPGQRVTISCTGSSSNIGAYYVHWYQQVPGTAPRLLI
FDNDNRPSGVPDRFSASKSGTSASLAIIGLQAEDEAEYYCQSVDYSLGDGVVFGGGT
KLTVL
(SEQ ID NO: 100)
QVQLVESGPGLVKPSETLSLTCAVSGGSISSNNWWSWVRQTPGKGLEW
IGEIYHSGSTNYNPSLKSRVTISVDKSKNQFSLKLSSVTAADTAVYYCANLRGQWGQG
TLVTVSS
>hFM46
(SEQ ID NO: 148)
QSVLTQPPSVSGAPGQRVTISCTGSSSNIGAGYDVHWYQQLPGTAPKV
LIYGNSNRPSGVPDRFSGSKSGTSASLAITGLQAEDEADYYCQSYDSSLSGSVVFGGG
TKLTVL
(SEQ ID NO: 147)
QVQLQQWGPGLVKPSGTLSLTCAVSGGSISSSNWWSWVRQFPGKGLE
WIGEMSHTGSTNYNPSFKSRVTISVDKSKNQFSLKLSPVTAADTAVYYCARGSTGAF
DVWGQGTMVTVSS
>hFM47
(SEQ ID NO: 134)
QAVLTQPSSVSGAPGQRVTISCTGSSSNIGAGYDVHWYQQLPGTAPKL
LIYGNRNRPSGVPDRFSGSKSGTSASLAITGLQAEDEADYYCQSYDSSLSGSKVFGTG
TKVTVL
(SEQ ID NO: 105)
QLQLQESGPGLVKPSETLSLTCTVSGGSISSGNYWSWVRQSPEKGLEWI
GEISHSGITNYNPSLQSRVTIALDKSKNHFSLNVNSVTAADTAVYYCVGTLRTWFDY
WGQGTLVTVSS
>hFM48
QSVLTQPPSVSAAPGQKVTISCSGSSSNIGNYYVSWYQQLPGTAPKLLI
YDNNKRPSGIPDRFSGSKSGTSATLGVTGLQTGDEADYYCGTWDSSLSAYVFGTGTK
VTVQ
(SEQ ID NO: 142)
QVQLQESGPGLVKSSETLSLICAVSGGSISSNNWWSWVRQPPGKGLEW
IGEIHHSGTTINYNPSLKSRVTISVDKSKNQFSLQLNSVTPEDTAVYFCARSASGAFDI
WGQGTMVTVSS
>hFM49
(SEQ ID NO: 143)
QAVLTQPSSVSGAPGQRVTISCTGSSSNIGAGYDVQWYQQLPGTAPKL
LIYADNNRPSGVPDRFSGSKSGTSASLAITGLQAEDEADYYCQSYDSSLSGSKVFGTG
TKVTVP
(SEQ ID NO: 144)
QVQLQESGPGLVELSGTLSLTCAVSGVSISSRNWWSWVRQTPGKGLE
WIGEISHSGIPNYNPSFVSRVTISIDRSQNQFSLKLTSMTAADTAVYYCARLYSGYGHG
MDVWGQGTTVTVSS
>hFM51
(SEQ ID NO: 145)
QAVLTQPSSVSGAPGQRVTISCTGSSSNIGAGYDVHWYQQLPGTAPKL
LIYGNSNRPSGVPDRFSGSKSGTSASLAITGLQAEDEADYYCQSYDSSLSGLYVFGTG
TKVTVL
(SEQ ID NO: 179)
QLQLQESGPGLVKPSGTLSLNCAVSGVSISSTNWWSWVRQFPGKGLE
WIGEINHSGTTNYNPSLKSRVTISVDTSKNQFSLQLNSVTPEDTAVYFCAQHLTVWGQ
GTLVTVSS
>hFM52
(SEQ ID NO: 164)
QAVLTQPSSLSGAPGQRVTISCTGSSSNIGAGYDVHWYQQLPGTAPKLL
ISGNRNRPSGVPDRFSGSKSGTSASLAITGLQAEDEADYYCQSYDSSLSGSFVFGTGT
KVTVL
(SEQ ID NO: 165)
QLQLQESGPGLVEPSGTLSLTCAVSGVSISTRNWWSWVRQTPGKGLEW
IGEISHSGIPNYNPSFVSRVTISVDASKNQLSLKLTSVTVADTAVYYCSRKGVDAFDIW
GQGTMVTVSS
>hFM53
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNED
YWGQGTLVTVSS
>hFM57
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM58
(SEQ ID NO: 143)
QAVLTQPSSVSGAPGQRVTISCTGSSSNIGAGYDVQWYQQLPGTAPKL
LIYADNNRPSGVPDRFSGSKSGTSASLAITGLQAEDEADYYCQSYDSSLSGSKVFGTG
TKVTVP
QVQLQESGPGLVEPSGTLSLTCAVSGVSISSRNWWSWVRQTPGKGLE
WIGEISHSGIPNYNPSFVSRVTISIDRSQNQFSLKLTSMTAADTAVYYCARLYSGYGHG
MDVWGQGTTVTVSS
>hFM59
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNFD
YWGQGTLVTVSS
>hFM60
QSVVTQPPSVSGAPGQRVTISCTGSSSNIGARYDVHWYQQLPGGAPKL
LIHSNSNRPSGVPDRFSGSKSGTSATLDITGLQTGDEADYYCGTWDSSLNSYVFGTGT
KVTVL
QVQLQQWGAGLLKPSETLSLTCAVYGGSFSGYYWSWIRQPPGKGLEW
IGEINHSGSTNYNPSLKSRVTISVDTSKNQFSLKLSSVTAADTAVYYCASLRRGYWGQ
GTLVTVSS
>hFM62
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNED
YWGQGTLVTVSS
>hFM64
(SEQ ID NO: 167)
QSVVTQPPSVSAAPGQKVTISCSGSSSNIGNYYVAWYQQVPGAAPKLLIYDTNKRPSG
IPDRFSGSKSGTSATLDITGLRTGDEADYYCGTWDSSLDTDVVFGGGTKLTVL
(SEQ ID NO: 168)
QVQLQESGPGLVKPSETLSLTCAVSGGSISSGSWWSWVRQAPGKGLE
WIGEISHSGTTTYNPSLKSRVTISLDKSTSHLSLSLKSVTAADTAVYYCARELGGGAYD
IWGQGTIVTVSS
>hFM65
(SEQ ID NO: 131)
QSVVTQPPSVSGAPGQRVTISCTGSSSNIGANYDVHWYQQLPGTAPKL
LIYGNSNRPSGVPDRFSGSKSGTSASLAITGLQAEDEADYYCQSYDSSLSGSSVFGGG
TKLTVL
(SEQ ID NO: 132)
QVQLQQSGPGLVKPSGTLSLTCDVSGGSISSRNWWTWVRQPPGKGLE
WIGEIYHSGSTNYNPSLESRVAMSVDKSRYQFSLRLSSVTAADTAVYYCARRRDGYF
DYWGQGTLVTVSS
>hFM66
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM68
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHNWWSWVRQTPGKGLE
WIGEISHSGITNYNPSLKSRVTISVDTSKNQLSLKLSSVTAADTAVYYCARGSTGAFDV
WGQGTMVTVSS
>hFM69
(SEQ ID NO: 137)
QAVLTQPSSVSGAPGQRVTISCTGINSNIGAGYDVHWYQQFPGAAPKV
LIYGNTNRPSGVPGRFSASKSGTSASLAITGLQAEDEADYYCQTYDSSLSGTWVFGG
GTKLTVL
(SEQ ID NO: 140)
QVQLQQWGPGLVKPSGTLSLTCAVSGVSISSSNWWSWVRQTPGKGLE
WIGEISHSGSPNYNPSLKSRVTISVDTSKNQFSLKLTSVTAADSAVYYCARGGGPIPHI
WGQGTTVTVSS
>hFM70
(SEQ ID NO: 137)
QAVLTQPSSVSGAPGQRVTISCTGTNSNIGAGYDVHWYQQFPGAAPKV
LIYGNTNRPSGVPGRFSASKSGTSASLAITGLQAEDEADYYCQTYDSSLSGTWVFGG
GTKLTVL
(SEQ ID NO: 140)
QVQLQQWGPGLVKPSGTLSLTCAVSGVSISSSNWWSWVRQTPGKGLE
WIGEISHSGSPNYNPSLKSRVTISVDTSKNQFSLKLTSVTAADSAVYYCARGGGPIPHI
WGQGTTVTVSS
>hFM71
(SEQ ID NO: 141)
QSVLTQPPSVSAAPGQKVTISCSGSSSNIGNYYVSWYQQLPGTAPKLLI
YDNNKRPSGIPDRFSGSKSGTSATLGVTGLQTGDEADYYCGTWDSSLSAYVFGTGTK
VTVQ
(SEQ ID NO: 142)
QVQLQESGPGLVKSSETLSLICAVSGGSISSNNWWSWVRQPPGKGLEW
IGEIHHSGTTINYNPSLKSRVTISVDKSKNQFSLQLNSVTPEDTAVYFCARSASGAFDI
WGQGTMVTVSS
>hFM72
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM73
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHNWWSWVRQTPGKGLE
WIGEISHSGITNYNPSLKSRVTISVDTSKNQLSLKLSSVTAADTAVYYCARGSTGAFDV
WGQGTMVTVSS
>hFM74
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM75
(SEQ ID NO: 149)
QAVLTQPSSVSGAPGQRVTISCTGSSSNIGAGYDVNWHQQFPGTAPKV
LIFGDNARPSGVPDRFYASKSGTSASLTIIGVQSDDEADYYCSTWDDSLNAVVFGGGT
TLTVL
QLQESGPGLVKPSGTLSLTCAVSGGSISSGNWWSWVRQPPGRGLEWIG
EISHSGTINYNPSLKSRVTISVDTSKNQFSLKLSSVTAADTAVYYCARRADGYFQPWG
QGTLVTVSS
>hFM76
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM77
(SEQ ID NO: 129)
QAVLTQPSSVSGAPGQRVTISCTGSSSNIGAGYDVHWYQQLPGTAPKLLIYSNTNRPS
GVPDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEW
IGEISHSGIPNYNPSLASRVTISIDRSQNQFSLKLTSVTAADTAVYYCARGGGRFDFWG
QGTLVTVSS
>hFM78
(SEQ ID NO: 162)
QAVLTQPSSVSGAPGQRVAISCTGSSSNIGAGYDVQWYQQLPGTAPKLLIYGNSNRPS
GVPDRFSGSKSGTSASLAITGLQAEDEADYYCQSYDSSLSGLRVFGGGTKLTVL
(SEQ ID NO: 163)
QLQLQESGPGLVKPSQTLSLTCTVSGASISSSNWWSWVRQTPGKGLEW
IGEISHSGSPNYNPSLKSRVTISVDTSKNQFSLKVSSVTAADTAVYYCAREPRYWGQG
TLVTVSS
>hFM80
(SEQ ID NO: 164)
QAVLTQPSSLSGAPGQRVTISCTGSSSNIGAGYDVHWYQQLPGTAPKLL
ISGNRNRPSGVPDRFSGSKSGTSASLAITGLQAEDEADYYCQSYDSSLSGSFVFGTGT
KVTVL
(SEQ ID NO: 165)
QLQLQESGPGLVEPSGTLSLTCAVSGVSISTRNWWSWVRQTPGKGLEW
IGEISHSGIPNYNPSFVSRVTISVDASKNQLSLKLTSVTVADTAVYYCSRKGVDAFDIW
GQGTMVTVSS
>hFM81
(SEQ ID NO: 167)
QSVVTQPPSVSAAPGQKVTISCSGSSSNIGNYYVAWYQQVPGAAPKLLIYDTNKRPSG
IPDRFSGSKSGTSATLDITGLRTGDEADYYCGTWDSSLDTDVVFGGGTKLTVL
(SEQ ID NO: 168)
QVQLQESGPGLVKPSETLSLTCAVSGGSISSGSWWSWVRQAPGKGLE
WIGEISHSGTTTYNPSLKSRVTISLDKSTSHLSLSLKSVTAADTAVYYCARELGGGAYD
IWGQGTIVTVSS
>hFM82
(SEQ ID NO: 169)
QSVLTQPPSVSAAPGQKVTISCSGSSSNIGNYHVSWYQQLPGTAPKLLI
YDNNKRPSGIPDRFSGSKSGTSATLDITGLQTGDEADYYCGTWDSSLSAVLFGRGTKL
TVL
(SEQ ID NO: 170)
QLQLQESGPGLVKPSGTLSLTCAVSGGSISSGYYWSWIRQPPGKGLEWI
GEINHSGSTNYNPSLKSRVTISVDTSKNQFSLKLSSVTAADTAVYYCARLRGSGRWGQ
GTLVTVSS
>hFM84
(SEQ ID NO: 171)
QAVLTQPSSVSGAPGQRLTISCTGSTSNIGAGYDVQWYQKLPGAAPKL
LVYGNTNRPSGVPDRFSGSKSGTSASLAITGLQAEDEGHYYCQSYDSSLSGWVVFGG
GTKLTVL
(SEQ ID NO: 172)
QLVQSGPGLVKPSGILSLTCAVSGGSITSSNWWSWVRQPPGKGLEWIGE
ISHSGSTNYNPSLKSRVTMSVDKSKNQLSLKLSSLTAADTAVYYCARGGSSLPIWGQG
TTVTVSS
>hFM85
(SEQ ID NO: 173)
QAVLTQPSSVSGAPGQTVTISCTGSSSNIGADYDVHWYQQLPGTAPKLLIYGNNNRPS
GVPDRFSGSKSGASASLAITGLQADDEADYYCHSYDSTRSGLYIFGTGTRVIV
(SEQ ID NO: 174)
QLQLQESGPGLVKPSGTLSLTCAVSGVSISTTNWWSWVRQPPGKGLEW
IGEISHSGITNYNPSLQSRVTISVDKSKNQFSLKLSSVTAEDTAVYYCTTGSSGYWGQG
TLVTVSS
>hFM87
(SEQ ID NO: 175)
QSVLTQPPSVSGAPGQRVTISCTGSSSNIGADYDVQWYQQLPGTAPKLL
IYANNNRPSGVPDRFSGSKSGTSASLAITGLQAEDEADYYCQSYDSSLSGLRVFGGGT
KLTVL
(SEQ ID NO: 176)
QVQLQQWGAGLLKPSETLSLTCAVYGGSFSGYYWSWIRQPPGKGLEW
IGEINHSGSTNYNPSLKSRVTISVDTSKNQFSLKLSSVTAADTAVYYCARSGRRSSWID
YWGQGTLVTVSS
>hFM88
(SEQ ID NO: 125)
QAVLTQPSSVSAAPGQKVTISCSGSDSNIGNYYVWWYQQLPGAAPKLLIYDNHRRPS
GVPDRFSGSKSGTSATLDITGLQTGDEADYYCGTWDGSLTGYVFGPGTKVTVL
(SEQ ID NO: 126)
QVQLQQWGAGLLKPSETLSLTCAVYGGSFSGYYWSWIRQPPGKGLEW
IGEINHSGSTNYNPSLKSRATISVDTSKNQFSLKLSSVTAADTAVYYCARGRVRGRWL
PYYWGQGTLVTVSS
>hFM90
(SEQ ID NO: 137)
QAVLTQPSSVSGAPGQRVTISCTGTNSNIGAGYDVHWYQQFPGAAPKV
LIYGNTNRPSGVPGRFSASKSGTSASLAITGLQAEDEADYYCQTYDSSLSGTWVFGG
GTKLTVL
(SEQ ID NO: 140)
QVQLQQWGPGLVKPSGTLSLTCAVSGVSISSSNWWSWVRQTPGKGLE
WIGEISHSGSPNYNPSLKSRVTISVDTSKNQFSLKLTSVTAADSAVYYCARGGGPIPHI
WGQGTTVTVSS
>hFM91
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM92
(SEQ ID NO: 160)
DIQLTQSPSSLSASVGDRVTITCRASQNINNFLNWYQQKPGNVPKLLIY
AASSLQSGVPSRFSGSGSGTDFTLTISSLQPEDFATYYCQQSYSPLSTFGQGTKVEIK
(SEQ ID NO: 161)
QVQLVQSGAEVKKPGSSVKVSCKASGYTFTGYYMHWVRQAPGQGLE
WMGRINPNSGGTNYAQKFQGRVTMTRDTSISTAYMELSRLRSDDTAVYYCARSVSV
VTDAFDIWGQGTTVTVSS
>hFM93
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM94
(SEQ ID NO: 153)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKVTVL
(SEQ ID NO: 154)
QVQLQESGPGLVKPSGTLSLTCDVSGGSISSNNWWSWVRQSPGKGLE
WIGEIIHTGRTNYNPSLTSRVTILIDKSKNQFSLKLTSVTPEDTALYYCARLRGPFDIWG
QGTMVTVSS
>hFM96
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHNWWSWVRQTPGKGLE
WIGEISHSGITNYNPSLKSRVTISVDTSKNQLSLKLSSVTAADTAVYYCARGSTGAFDV
WGQGTMVTVSS
>hFM97
(SEQ ID NO: 139)
QAVLTQPSSVSGAPGQRVTISCTGSSTNIGADFDVHWYQQLPGTAPKLL
IHGDNNRPSGVPDRFSGSKSGTSAYLAISGLQAEDEADYYCQTYDSRSSGSRVFGGGT
KVTVL
(SEQ ID NO: 107)
QLQLQESGPGLVKPSGTLSLTCAVSGVSISSRNWWSWVRQTPGKGLE
WIGEISHSGSTNYNPSLKSRVTISVDTSKNQFSLKLSSVTAADTAVYYCARNAGDIWG
QGTMVTVSS
>hFM98
(SEQ ID NO: 158)
DVVMTQSPLSLAVTLGQPASISCRSSQSLLHSSGYKFLNWYLQKPGQSPQLLIYLGSN
RASGVPDRFSGSGSGTDFTLKISRVEAEDVGVYYCMQALQSPTFGGGTKVEIK
(SEQ ID NO: 159)
EVQLVESGGGLVQPGGSLRLSCTASGFTFSSFSMNWVRQAPGKGPEW
VSAISGSGGGTYYADSVKGRFTISRDNAKNSLYLQMSSLRAEDTAVYYCARVRVPQA
FDIWGQGTMVTVSS
>hFM99
(SEQ ID NO: 121)
QAVLTQPSSVSGAPGQRVTISCTGSRSNIGANYDVHWYQQLPGTAPKL
LIFNDNIRPSGVPDRFSGSKSGTSASLAITGLQAEDEADYYCQSYDSSLSGSVVFGGG
TKLTVL
(SEQ ID NO: 122)
QVQLQESGPGLVKPSGTLSLTCAVSGVSISTTNWWSWVRQPPGKGLE
WIGEISHSGITNYNPSLQSRVTIALDKSKNHFSLNVNSVTAADTAVYYCASYWLGNED
YWGQGTLVTVSS
>hFM100
(SEQ ID NO: 123)
QSVLTQPPSVSGAPGQRVTISCTGSSSNIGAGYEVHWYQQLPGRAPRLL
IFDNNNRPSGVPDRFSASKSGTSASLAITGLRAEDEGDYYCQSYDSKRTPPYVFGTGT
RVTVL
(SEQ ID NO: 124)
QVQLQQSGAEVKKPGASVKVSCKASGYSFSKYGMSWVRQAPGQGLE
WMGWINAGNGDTKYSQKFQGRVTITRDTSASTAYMELSSLKYEDTAIYYCARRLSY
YGMDVWGQGTTVTVSS
>hFM101
(SEQ ID NO: 125)
QAVLTQPSSVSAAPGQKVTISCSGSDSNIGNYYVWWYQQLPGAAPKLLIYDNHRRPS
GVPDRFSGSKSGTSATLDITGLQTGDEADYYCGTWDGSLTGYVFGPGTKVTVL
(SEQ ID NO: 126)
QVQLQQWGAGLLKPSETLSLTCAVYGGSFSGYYWSWIRQPPGKGLEW
IGEINHSGSTNYNPSLKSRATISVDTSKNQFSLKLSSVTAADTAVYYCARGRVRGRWL
PYYWGQGTLVTVSS
>hFM102
(SEQ ID NO: 155)
QAVLTQPSSVSGAPGQRVIISCTGSSSNIGAGYDVHWYQQLPGTAPKLL
IYGNTNRPSGVPDRFSGSKSGTSASLAITGLQAEDEADYYCQSYDSSLSGNYVFGPGT
KVTVL
(SEQ ID NO: 127)
QVQLQESGPGLVKPSETLSLTCAVSGVSISSHNWWSWVRQTPGKGLE
WIGEISHSGITNYNPSLKSRVTISVDTSKNQLSLKLSSVTAADTAVYYCARGSTGAFDV
WGQGTMVTVSS
>hFM103
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM104
(SEQ ID NO: 112)
QSVVTQPPSVSGAPGQRVTISCTGSSSNIGAGYDVHWYQQLPGTAPKL
LIYGNSNRPSGVPDRFSGSKSGTSASLAITGLQAEDEADYYCQSYDSSLSGFWVFGG
GTKLTVL
(SEQ ID NO: 113)
QVQLQESGPGLVKPSGTLSLTCAVSGGSISSSNWWSWVRQPPGKGLEW
IGEISHSGSTNYNPSLKSRVTISVDNSKNQFSLQLSSVTAADTAVYYCVSNASGRRGFA
WGQGTLVTVSS
>hFM105
(SEQ ID NO: 149)
QAVLTQPSSVSGAPGQRVTISCTGSSSNIGAGYDVNWHQQFPGTAPKVLIFGDNARPS
GVPDRFYASKSGTSASLTIIGVQSDDEADYYCSTWDDSLNAVVFGGGTTLTVL
(SEQ ID NO: 150)
VQLQESGPGLVKPSGTLSLTCAVSGGSISSGNWWSWVRQPPGRGLEWI
GEISHSGTINYNPSLKSRVTISVDTSKNQFSLKLSSVTAADTAVYYCARRADGYFQPW
GQGTLVTVSS
>hFM106
(SEQ ID NO: 148)
QSVLTQPPSVSGAPGQRVTISCTGSSSNIGAGYDVHWYQQLPGTAPKV
LIYGNSNRPSGVPDRFSGSKSGTSASLAITGLQAEDEADYYCQSYDSSLSGSVVFGGG
TKLTVL
(SEQ ID NO: 147)
QVQLQQWGPGLVKPSGTLSLTCAVSGGSISSSNWWSWVRQFPGKGLE
WIGEMSHTGSTNYNPSFKSRVTISVDKSKNQFSLKLSPVTAADTAVYYCARGSTGAF
DVWGQGTMVTVSS
>hFM107
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM108
(SEQ ID NO: 151)
VLTQPPSVSGAPGQRVTISCTGSDSNIGAGYDVHWYQQYPGIAPKLLIY
AHHKRPSGVPDRFSGSTSGTSASLAITGLQAEDEADYYCQSYDSSLSGHYVFGTGTQ
VSVL
(SEQ ID NO: 152)
QVQLQESGPGLVKPSETLSLTCAVSGGSISSNNWWSWVRQTPGKGLE
WIGEIYHSGNTNYNPSLKSRVTISVDKSKNQFSLKLNSVTAADTAVYYCARGRQGAF
DPWGQGTLVTVSS
>hFM109
(SEQ ID NO: 129)
QAVLTQPSSVSGAPGQRVTISCTGSSSNIGAGYDVHWYQQLPGTAPKLLIYSNTNRPS
GVPDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEW
IGEISHSGIPNYNPSLASRVTISIDRSQNQFSLKLTSVTAADTAVYYCARGGGRFDFWG
QGTLVTVSS
>hFM110
(SEQ ID NO: 112)
QSVVTQPPSVSGAPGQRVTISCTGSSSNIGAGYDVHWYQQLPGTAPKL
LIYGNSNRPSGVPDRFSGSKSGTSASLAITGLQAEDEADYYCQSYDSSLSGFWVFGG
GTKLTVL
(SEQ ID NO: 113)
QVQLQESGPGLVKPSGTLSLTCAVSGGSISSSNWWSWVRQPPGKGLEW
IGEISHSGSTNYNPSLKSRVTISVDNSKNQFSLQLSSVTAADTAVYYCVSNASGRRGFA
WGQGTLVTVSS
>hFM112
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 114)
QVQLQESGPGLVELSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM113
(SEQ ID NO: 115)
QSVLTQPPSVSGAPGQRITISCTGSSSNIGAGYDVQWYQQVPGKAPKH
LIYGNSNRPSGVPDRFSGSKSGTSASLAITGLQAEDEADYFCQSYDSSLSGYVVFGGG
TKLTVL
(SEQ ID NO: 116)
QVQLQESGPGLVKPSETLSLTCAVSGGSISSSNWWSWVRQPPGKGLEW
IGEISHTGSPNYNPSLASRVTISMDKSKNQFSLNLRSVTAADTSVYYCARYGRGAFDI
WGQGTMVTVSS
>hFM114
(SEQ ID NO: 117)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKLTVL
(SEQ ID NO: 118)
QVQLVESGPGLVKPSGTLSLTCAVSGGSISSSDWWSWVRQPPGKGLEW
IGEMRLSGTTNYNPSLKSRVAISMDKSKNHFSLNLSSVTAADTAVYYCANADYYTHH
YWGQGTLVTVSS
>hFM115
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM116
(SEQ ID NO: 153)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKVTVL
(SEQ ID NO: 154)
QVQLQESGPGLVKPSGTLSLTCDVSGGSISSNNWWSWVRQSPGKGLE
WIGEIIHTGRTNYNPSLTSRVTILIDKSKNQFSLKLTSVTPEDTALYYCARLRGPFDIWG
QGTMVTVSS
>hFM117
(SEQ ID NO: 117)
QAVLTQPSSVSGAPGQRVTISCTGSSSNIGAGYDVQWYQQLPGTAPKL
LIYADSNRPSGVPDRFSGSKSGTSASLAITGLQAEDEADYYCQSYDSSLSGSKVFGTG
TKLTVL
(SEQ ID NO: 118)
QVQLVESGPGLVKPSGTLSLTCAVSGGSISSSDWWSWVRQPPGKGLEW
IGEMRLSGTTNYNPSLKSRVAISMDKSKNHFSLNLSSVTAADTAVYYCANADYYTHH
YWGQGTLVTVSS
>hFM118
(SEQ ID NO: 135)
VLTQPPSVSGAPGQRVTISCTGGSTNIGAGYDVHWYQQLPGTAPKLLIY
GNNNRPSGVPDRFSGSQSGASASLAITGLQADDEADYYCQSYDSRLDGSKVFGTGT
KVTVL
(SEQ ID NO: 136)
QVQLQESGPGLVKPSETLSLTCAVSGVSISSHNWWSWVRQTPGKGLE
WIGEMSHSGIPNYNPSLESRVTISLDKSKNQFSLILRSVTAADTAMYYCVGGSGSYSY
WGQGTLVTVSS
>hFM120
(SEQ ID NO: 112)
QSVVTQPPSVSGAPGQRVTISCTGSSSNIGAGYDVHWYQQLPGTAPKL
LIYGNSNRPSGVPDRFSGSKSGTSASLAITGLQAEDEADYYCQSYDSSLSGFWVFGG
GTKLTVL
(SEQ ID NO: 113)
QVQLQESGPGLVKPSGTLSLTCAVSGGSISSSNWWSWVRQPPGKGLEW
IGEISHSGSTNYNPSLKSRVTISVDNSKNQFSLQLSSVTAADTAVYYCVSNASGRRGFA
WGQGTLVTVSS
>hFM121
(SEQ ID NO: 128)
QSVLTQPPSVSAAPGQKVTISCSGSTSNVGNYYVAWYQKLPGEAPKVL
IHDNNRRPSGIPVRFSGSKSGTSATLGITGLQTGDEAEYYCAVWDSSLNGYVFGGGTK
LTVL
(SEQ ID NO: 111)
QVQLQQWGAGLLKPSETLSLTCAVYGGSFSGYYWSWIRQPPGKGLEW
IGEINHSGSTNYNPSLKSRVTISVDTSKNQFSLKLSSLTAADTAVYYCARVNGGESDY
WGQGTLVTVSS
>hFM122
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM123
(SEQ ID NO: 129)
QAVLTQPSSVSGAPGQRVTISCTGSSSNIGAGYDVHWYQQLPGTAPKLLIYSNTNRPS
GVPDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEW
IGEISHSGIPNYNPSLASRVTISIDRSQNQFSLKLTSVTAADTAVYYCARGGGRFDFWG
QGTLVTVSS
>hFM124
(SEQ ID NO: 131)
QSVVTQPPSVSGAPGQRVTISCTGSSSNIGANYDVHWYQQLPGTAPKL
LIYGNSNRPSGVPDRFSGSKSGTSASLAITGLQAEDEADYYCQSYDSSLSGSSVFGGG
TKLTVL
(SEQ ID NO: 133)
QVQLQQSGPGLVKPFGRPCPLTCDVSGGSISSRNWWTWVRQPPGKGL
EWIGEIYHSGSTNYNPSLESRVAMSVDKSRYQFSLRLSSVTAADTAVYYCARRRDGYF
DYWGQGTLVTVSS
>hFM125
(SEQ ID NO: 131)
QSVVTQPPSVSGAPGQRVTISCTGSSSNIGANYDVHWYQQLPGTAPKL
LIYGNSNRPSGVPDRFSGSKSGTSASLAITGLQAEDEADYYCQSYDSSLSGSSVFGGG
TKLTVL
(SEQ ID NO: 132)
QVQLQQSGPGLVKPSGTLSLTCDVSGGSISSRNWWTWVRQPPGKGLE
WIGEIYHSGSTNYNPSLESRVAMSVDKSRYQFSLRLSSVTAADTAVYYCARRRDGYF
DYWGQGTLVTVSS
>hFM126
(SEQ ID NO: 103)
QSVLTQPPSVSGAPGQRVTISCTGSSSNIGAGYDVHWYQQLPGTAPKLL
IYDNINRPSGVPDRFSGSKSGASASLAITGLQAEDEADYYCQSYDSSLSGLRVFGTGT
KVTVL
(SEQ ID NO: 104)
QVQLQESGPGLVEPSGTLSLTCAVSGVSISSRNWWSWVRQTPGKGLE
WIGEINHSGSTNYNPSLKSRVTISVDTSKNQFSLKLSSVTAADTAVYYCARGGSDMDV
WGKGTMVTVSS
>hFM127
(SEQ ID NO: 134)
QAVLTQPSSVSGAPGQRVTISCTGSSSNIGAGYDVHWYQQLPGTAPKL
LIYGNRNRPSGVPDRFSGSKSGTSASLAITGLQAEDEADYYCQSYDSSLSGSKVFGTG
TKVTVL
(SEQ ID NO: 105)
QLQLQESGPGLVKPSETLSLTCTVSGGSISSGNYWSWVRQSPEKGLEWI
GEISHSGITNYNPSLQSRVTIALDKSKNHFSLNVNSVTAADTAVYYCVGTLRTWFDY
WGQGTLVTVSS
>hFM128
(SEQ ID NO: 106)
QAVLTQPSSVSGAPGQRVTISCTGSSTNIGAGFDVHWYQQLPGTAPKLL
IYGDKNRPSGVPDRFSGSKSGTSAYLAITGLQAEDEADYYCQTYDSRLSGSKVFGGG
TKVTVL
(SEQ ID NO: 107)
QLQLQESGPGLVKPSGTLSLTCAVSGVSISSRNWWSWVRQTPGKGLE
WIGEISHSGSTNYNPSLKSRVTISVDTSKNQFSLKLSSVTAADTAVYYCARNAGDIWG
QGTMVTVSS
>hFM130
(SEQ ID NO: 108)
QSVLTQPPSVSAAPGQKVTISCSGSSSDIGNNFVSWYQQLPGTAPKRLI
YDNSKRPSGIPERFSGSKSGTSATLGITGLQTGDEADYYCGAWDTSLSAYVFGTGTKV
TVL
QVQLQQWGPGLVKPSETLSLTCAVYGGSFSGYYWSWIRQPPGKGLEW
IGEINHSGSTNYNPSLKSRVTISVDTSKNQFSLKLSSVTAADTAVYYCARYSNYRHYY
YGMDVWGQGTLVTVSS

While the invention has been described through illustrative examples, routine modifications will be apparent to those skilled in the art, which modifications are intended to be within the scope of the present disclosure.

1. A binding partner that specifically binds to a drug that is used as a drug component of an antibody-drug conjugate (ADC).

2. The binding partner of claim 1, wherein the binding partner preferentially binds to the drug when it is removed from the ADC, relative to binding of the binding partner to the drug when the drug is present in the ADC.

3. The binding partner of claim 2, wherein the binding partner comprises an antibody or antigen binding fragment thereof, and wherein the antigen binding fragment is optionally a Fab, Fab′, or an F(ab′)2.

4. The binding partner of claim 3, wherein the drug is monomethyl auristatin E (MMAE) or monomethyl auristatin F (MMAF).

5. The binding partner of claim 4, wherein the binding partner comprises a light chain comprising the sequence:
(SEQ ID NO: 1)
DIVMTQSPSSLSASVGDRVTITCRASQDVGTAVAWYQQKPGQSPKLLIYW
ASTRHTGVPDRFSGSGSGTDFTLTISGLQSEDEADYFCQQYSNYPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
and a heavy chain comprising the sequence:
(SEQ ID NO: 2)
EVQLVESGGGLVKPGGSLKLSCAASGFTFSGYAMSWFRQAPEKRLEWVAT
ISSGGSYTYYLDSVKGRFTISRDNSKNTLYLQMSSLRSEDTAVYYCLASL
ITTDYFEYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSC
wherein optionally the sequence of the light chain and/or the heavy chain comprise at least one amino acid substitution,
wherein the at least one amino acid substitution in the light chain is selected from the group consisting of H55Y, R24H, and L60A;
and/or wherein the at least one amino acid substitution in the sequence of the heavy chain is selected from the group consisting of F27L, R44G, E42D, E42G, L61A and I101F.

6. The binding partner of claim 5, wherein the light chain comprises the H55Y amino acid substitution or wherein the heavy chain comprises the F27L mutation.

7. The binding partner of claim 6, wherein the light chain comprises the H55Y amino acid substitution and wherein the heavy chain comprises the F27L mutation.

8. The binding partner of claim 7, wherein the light chain comprises the sequence
(SEQ ID NO: 3)
DIVMTQSPSSLSASVGDRVTITCRASQDVGTAVAWYQQKPGQSPKLLIYW
ASTRYTGVPDRFSGSGSGTDFTLTISGLQSEDEADYFCQQYSNYPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
and the heavy chain comprises the sequence:
(SEQ ID NO: 4)
EVQLVESGGGLVKPGGSLKLSCAASGLTFSGYAMSWFRQAPEKRLEWVAT
ISSGGSYTYYLDSVKGRFTISRDNSKNTLYLQMSSLRSEDTAVYYCLASL
ITTDYFEYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSC

9. The binding partner of claim 8, wherein the heavy chain or the light chain, or a combination thereof, is adapted to further bind to a substance in blood in an individual to which the binding partner is administered but is not the drug.

10. The binding partner of claim 9, wherein binding of the binding partner to the substance in blood increases half-life of the binding partner in the circulation of the individual, and wherein optionally the heavy chain is adapted to bind to the substance.

11. A method for reducing non-target toxicity of an antibody-drug conjugate (ADC) comprising administering to an individual who has received or is concurrently receiving the ADC a binding partner of claim 1.

12. The method of claim 11, wherein the binding partner preferentially binds to the drug when it is removed from the ADC, relative to binding of the binding partner to the drug when the drug is present in the ADC.

13. The method of claim 12, wherein the binding partner comprises an antibody or antigen binding fragment thereof, and wherein the antigen binding fragment is optionally a Fab, Fab′, or an F(ab′)2.

14. The method of claim 13, wherein the drug is monomethyl auristatin E (MMAE) or monomethyl auristatin F (MMAF).

15. The method of claim 14, wherein the binding partner comprises a light chain comprising the sequence:
(SEQ ID NO: 97)
DIVMTQSPSSLSASVGDRVTITCRASQDVGTAVAWYQQKPGQSPKLLIYW
ASTRHTGVPDRFSGSGSGTDFTLTISGLQSEDEADYFCQQYSNYPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
and a heavy chain comprising the sequence:
(SEQ ID NO: 2)
EVQLVESGGGLVKPGGSLKLSCAASGFTFSGYAMSWFRQAPEKRLEWVAT
ISSGGSYTYYLDSVKGRFTISRDNSKNTLYLQMSSLRSEDTAVYYCLASL
ITTDYFEYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSC
wherein optionally the sequence of the light chain and/or the heavy chain comprise at least one amino acid substitution,
wherein the at least one amino acid substitution in the light chain is selected from the group consisting of H55Y, R24H, and L60A;
and/or wherein the at least one amino acid substitution in the sequence of the heavy chain is selected from the group consisting of F27L, R44G, E42D, E42G, L61A and I101F.

16. The method of claim 15, wherein the light chain comprises the H55Y amino acid substitution or wherein the heavy chain comprises the F27L mutation.

17. The method of claim 16, wherein the light chain comprises the H55Y amino acid substitution and wherein the heavy chain comprises the F27L mutation.

18. The method of claim 17, wherein the light chain comprises the sequence
(SEQ ID NO: 3)
DIVMTQSPSSLSASVGDRVTITCRASQDVGTAVAWYQQKPGQSPKLLIYW
ASTRYTGVPDRFSGSGSGTDFTLTISGLQSEDEADYFCQQYSNYPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
and the heavy chain comprises the sequence:
(SEQ ID NO: 4)
EVQLVESGGGLVKPGGSLKLSCAASGLTFSGYAMSWFRQAPEKRLEWVAT
ISSGGSYTYYLDSVKGRFTISRDNSKNTLYLQMSSLRSEDTAVYYCLASL
ITTDYFEYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSC.

19. The method of claim 18, wherein the heavy chain or the light chain, or a combination thereof, is adapted to further bind a substance in an individual to which the binding partner is administered but is not the drug.

20. The method of claim 19, wherein binding of the binding partner to the substance increases half-life of the binding partner in the circulation of the individual, and wherein optionally the heavy chain is adapted to bind to the substance.

21. A binding partner comprising or consisting a pair of a light and a heavy chain, wherein the light chain and the and heavy chain sequences are selected from the sequences:
VH_E42G (Humanness)
(SEQ ID NO: 53)
EVQLVESGGGLVKPGGSLKLSCAASGFTFSGYAMSWFRQAPGKRLEWVATISSGGSYTY
YLDSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSC
VH_R44G (Humanness)
(SEQ ID NO: 54)
EVQLVESGGGLVKPGGSLKLSCAASGFTFSGYAMSWFRQAPEKGLEWVATISSGGSYTY
YLDSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSC
>VH_L60A (Humanness)
(SEQ ID NO: 55)
EVQLVESGGGLVKPGGSLKLSCAASGFTFSGYAMSWFRQAPEKRLEWVATISSGGSYTY
YADSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSC
>VH_F37V (Humanness)
(SEQ ID NO: 56)
EVQLVESGGGLVKPGGSLKLSCAASGFTFSGYAMSWVRQAPEKRLEWVATISSGGSYTY
YLDSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSC
VH_F27L (Affinity)
(SEQ ID NO: 4)
EVQLVESGGGLVKPGGSLKLSCAASGLTFSGYAMSWFRQAPEKRLEWVATISSGGSYTY
YLDSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSC
>C1B11
(SEQ ID NO: 57)
MDIVMTQSPSSLSASVGDRVTITCRASQDVGTAVAWYQQKPGQSPKLLIYRASTRHTGVP
DRFSGNGSGTDLTLTISGLQSEDEADYFCQQYSNYPYTFGGGTKLEIKRGGGGSGGGGS
GGGGSGGGGSEVQLVESGGGLVKPGGSLKLSCAASGFSFSGYAMSWFRQAPDKRLEWV
ATISSGGSYTYYLDSVKGRFTISRDNSKNTLYLQMSSLRSEDTAVYYCLASLITTDYFEYW
GQGTLVTVSS
>C1C8
(SEQ ID NO: 58)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRPEWV
ATISSGGSYTYYLDSVKGRFTISRDNNKNTLFLQMSSLRSEDTAVYYCLASMITTDYFEY
WGQGTLVTVSS
>C1D7
(SEQ ID NO: 59)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTELEIKRGGSGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMSSLRSEDTAVYYCLASLVTTDYFEY
WGQGTLVTVSS
>C1D10
(SEQ ID NO: 60)
MDIVMTQSTSSMRASVGDRVTITCRASQDVGTAVAWYQQKPGQSPKLLIYWASTRHTG
VPDRFSGSGSGTDFTLTISGLQSEDEADYFCQQYSNYPYTFGGGTKLEIKRGGGGSGGGG
SGGGGSGGGGSEVQLVESGGGLVKPGGSLKLSCAASGFTFSGYAMSWFRQAPEKRLEW
VATISSGGSYTYYLDSVKGRFTISRDNSKNTLYLQMSSLRSEDTAVYYCLASLITTDYFEY
WGQGTLVTVSS
>C1E2
(SEQ ID NO: 61)
MDIVMTQSPSSLSASVGDRVIFTCRTSQDVGTAVAWYQQKPGQSPKLLIYWASTRHTGVP
DRFSGSGSGTDFTLTISGLQSEDEADYFCQQYSNYPYTFGGGTELEVKRSGGGSGGGGS
GGGGSGGGGSEVQLVESGGGLVKPGGSLKLSCAASGFTFSGYAMSWFRQAPEKRLEWV
ATISSSGSYTYYLDSVKGRFTISRDNSKNTLYLQMSSLRSEDTAVYYCLASLMTTDYFEY
WGQGTLVTVSS
>C1G3
(SEQ ID NO: 62)
MDIVMTQSPSSLSASVGDRVTITCRASQDVGTAVAWYQQKPGQSPKLLIYWASTRHTGV
PDRFSGSGSGTDFTLTISGLQSEDEADYFCQQFSNYPYTFGGGTKLEIKRGGGGSGGGGS
GGGGSGGGGSEVQLVESGGGLVKPGGSLKLSCAASGLTFSGYAMSWFRQAPEKRLEWV
ATISSGGSYTYYLDSVKGRFTISRDNSKKTLYLQMSSLRSEDTAVYYCLASLITTDYFEYW
GQGTLVTVSS
>C2C2
(SEQ ID NO: 63)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSGVQLVESGGGLVKPGGSLKLSCAASGFTISGYAMSWFRQTPEKRLEWV
ATISSGGSYTYYLDSVKGRFTTSRDNSINTLYLQMSSLRSEDTAVYYCLASLFTTDYFEY
WGQGTLVTVNS
>C2C4
(SEQ ID NO: 64)
MDIVMTQSPSSLSASVGDRVTISCRASQDVGTAVAWYQQKPGQSPKLLIYWASTRHTGV
PDRFSGSGSGTDFTLTISGLQSEDEADYFCQQYSNYPYTFGGGSKLEIKRGGGGSGGGGS
GGGGSGGGGSEVQLVESGGGLVKPGGSLKLSCAASGFTFSGYAMSWFRQTPEKRLEWV
ATISSGGSYTYYLDSVKGRFTISRDNSKNTLYLQMSSLRSEDTAVYYCLASLITTDYFEYW
GQGTLVTVSS
>C2D11
(SEQ ID NO: 65)
MDIVMTQSPSSLSASVGDRVTITCRASQDVGTAVAWYQQKPGQSPKLLIYWASTRHTGV
QDRFSGSGSGTVFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPGKRLEWV
ATISSGGSYTYYLDSVKGRFTISRDNSKNTLYLQMSSLRSEDTAVYYCLASLITTDYFEYW
GQGTLVTVSS
>C2F5
(SEQ ID NO: 66)
MDIVMTQSPSSLGASVGDRVTITCRASQDIGTAVAWYQQKPGQSPKLLIYWASTRHTGVP
DRFSGSGSGTDFTLTISGLQSEDEADYFCQQYSNYPYTFGAGTKLEIRRGGGGSGGGGSG
GGGSGGGGSEVQLDESGGGLVKPGGSLKLSCAASGFTFSGYAMSWFRQAPDKRLEWVA
TISSGGSYTYYLDSVKGRFTISRDNSKNTLYLQMSSLRSEDTAVYYCLASLITTDYFEYW
GQGTLVTVSS
>DBG6
(SEQ ID NO: 67)
MDIVMTQSPSSLSTSVGDRVTITCRASQDVGTAVAWYQQKPGQSPKLLIYWASTRHTGV
PGRFSGSGSGTDFTLTISGLQSEDEADYFCQQYSNYPYTFGGGTKLEINRGGGGSGGGGS
GGGGSGGGGSEVQLVESGGGLVKPGGSLKLSCAASGFTFSGYAMSWFRQAPEKRLEWV
ATISSGGSYTYYLDSVKGRFTVSRDNSKNTLYLQMSSLRSEDTAVYYCLASLITTDYFEY
WGQGTLVTVSS
>DAD3
(SEQ ID NO: 68)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVERGGSLKLSCAASGFTFSGYAMSWFRQAPEKRLEWV
ATISSGGSYTYYLDSVKGRFTISRDNSKNTLYLQMSSLRSEDTAVYYCLASLLTTDYFEY
WGQGTLVTVSS
>DAD4
(SEQ ID NO: 69)
MDIVMTQSPSSLNASVGGRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGLTFSGYAMSWFRQAPEKRLEWV
GTISSGGSYTYYLDSVKGRFTISRDNSKNTLYLQMSSLRSEDTAVYYCLASLITTDYFEY
WGQGTLVTVSS
>DAG4
(SEQ ID NO: 70)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMSSLRSEDTAVYYCLASLITTDYLEY
WGQGTLVTVSS
>DAC2
(SEQ ID NO: 71)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTHYLDSVKGRFTISRDNSKNTLYLQMSSLRSEDTAVYYCLASLITTDYFEYW
GQGTLVTVSS
>DBF5
(SEQ ID NO: 72)
MDIVMTQSPSSQSASIGDRVTITCRASQDVGTAVAWYQQKPGQSPKLLIYWASTRHTGVP
DRFSGSGSGTDFTLTISGLQSEDEADYFCQQYSNYPYTFGGGTKLEIERGGGGSGGGGSG
GGGSGGGGSEVQLVESGGGLVKPGGSLKLSCAASGFTFSGYAMSWFRQAPEKRLEWVA
TISSGGSYTYYLDSVKGRFTISRDNSKNTLYLQTSSLRSEDTAVYYCLASLITTDYFEYWG
QGTLVTVSS
>DAD10
(SEQ ID NO: 73)
MDIVMTQSPSGLSASVGDRVTITCHASQDVGTAVAWYQQKPGQSPKLLIYWASTRHTGV
PDRFSGSGSGSDFTLTISGLQSEDEADYFCQQYSNYPYTFGGGTKLEIKRGGGGSGGGGS
GGGGSGGGGSEVQLFESGGGLVKPGGSLKLSCAASGFTFSGYAMSWFRQAPEKRPEWV
ATISSGGSYTYYLDSVKGRFTISRDNSKNTLYLQMSSLRSEDTAVYYCLASLITTDYFEYW
GQGTLVTVSS
>AC10
(SEQ ID NO: 74)
MDIVMTQSPSSLSASVGDRVTITCSASQDVGTAGAWYQQKPGQSPKLLIYWASTRHTGV
PDRFSGSGSGTDFTLTISGLQSEDEADYFCQQYSNYPYTFGGGTKLEIKRGGGGSGGGGS
GGGDSGSGGSEVQLVESGGGLVKRGGSLKLSCAASGFTLSGYAMSWFRQAPGKRPEWV
ATISSGGSYTYYLDSVKGRFTISRDNSKNTLYLQMSSLRSEDTAVYYCLASLITTDYFEYW
GQGTLVTVSS
>DBB10
(SEQ ID NO: 75)
MDIVMTQSPSSLSASVGDRVTFTCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DBD2
(SEQ ID NO: 76)
MDIVMTQSPSSLSASVGDRVTITCHASQDVGTAVAWYQQKPGQSPKLLIYWASTRHTGV
PDRFSGSGSGTDFNLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPDKRLEWV
ATISSGGGYTYYLDSVKGRFTISRDNSKNTLYLQMSSLRSEDTAVYYCLASLITTDYFEY
WGQGALVTVSS
>DBG2
(SEQ ID NO: 77)
MDIVMTQSPSSLSASVGDRVTITCRASQDVGTAVAWYQQKPGQSPKLLIYWASTRYTGV
PDRFSGSGSGTDFTLTISGLQSEDEADYFCQQYSNYPYTFGGGTKLEIKRGGGGSGGGGS
GGGGSGGGGSKVQLVESGGGLVKRGGSLKLSCAASGFTFSGYAMSWFRQAPEKRLEWV
ATISSGGSYTYYLDSVKGRFTISRDNSKNTLYLQLSSLRSEDTAVYYCLASLITTDYFEYW
GQGTLVTVSS
>DAD5
(SEQ ID NO: 78)
MDIVMTQSPSSLSASVGDRVTITCRASQDVGTAVAWYQQKPGQSPKLLIYWASTRYTGV
PDRFSGSGSGTDFTLTISGLQSEDEADYFCQQYSNYPYTFGGGTKLEIKRGGGGGGGGGS
GGGGSGGGGSEVQLVESGGGLVKPGGSLKLSCAASGLTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DAG2
(SEQ ID NO: 79)
MDIVMTQSPSSLSASVGDRVTITCRTSQDVGTAVAWYQQKPGQSPKLLIYWASTRHTGVP
DRFSGSGSGTDFTLTISGLQSEDEADYFCQQYSNYPYTFGAGTKLEIKRGGGGSGGGGSG
GGGSGGGGSEVQLVESGGGLVKPGGSLKLSCAASGLTFSGYAMSWFRQAPEKRLEWVA
TISSGGSYTYYLDSVKGRFTISRDNSKNTLYLQMSSLRSEDTAVYYCLASLITTDYFEYW
GQGTLVTVSS
>DAG7
(SEQ ID NO: 80)
MDIVMTQSPSSLSASVGDRVTITCRASQDVGTAVAWYRQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKGGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DAB6
(SEQ ID NO: 81)
MDIVMTQSPSSLSASVGDRVTIACRASQDVGTAVAWYQQKPGQSPKLLIYWASTRHTGV
PDRFSGSGSGTDFTLTISGLQSEDEADYFCQQYSNYPYTFGGGTKLEVKRGGGGSGGGG
SGSGGSGGGGSEVQLVESGGGLVKPGGSLKLSCAASGSTFSGYAMSWFRQAPDKRLEW
VATISSGGSYTYYLDSVKGRYTISRDNSKNTLYLQMSSLRSEDTAVYYCLASRITTDYFEY
WGQGTLVTVSS
>DAC3
(SEQ ID NO: 82)
MDIVMTQSPSSLSASVGDRVTITCRASQDVGAAVAWYKKKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DAC4
(SEQ ID NO: 83)
MDIVMTQSPSSLSASVGDRVTITCHASQDVGTAVAWYQQKPGQSPKLLIYWASTRHTGV
PDRFSGSGSGTDFTLTISGLQSEDEADYSCQQYSNYPYTFGGGTKLEIKRGGGGSGGGGS
GGGGSGGGGSEVQLVESGGGRVKPGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DAC9
(SEQ ID NO: 84)
MDIVMTQSPSSLSASVGDRVTITCHASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPDKRLEWV
ATISSGGSYTYYLDSVKGRFTISRDNSKNTLYLQMSSLRSEDTAVYYCLASLITTDYFEYW
GQGTLVTVSS
>DAD9
(SEQ ID NO: 85)
MDIVMTQSPSSLSASVGDRVTITCRASQDVGTAVAWYQQKPGQSPKLLIYWASTRHTGV
PGRFSGSGSGTDFTLTIRGLQSEDEADYFCQQYSNYPYTFGGGTKLDIKRGGGGSGGGGS
GGGGSGGGGSEVQLVESGGGLVKPGGSLKLSCAASGFTFSGYAMSWFRQAPDKRLEWV
ATISSGGSYTYYLDSVKGRFTISRDNSKNTLYLQMSSLRSEDTAVYYCLASLFTTDYFEY
WGQGTLVTVSS
>DAE2
(SEQ ID NO: 86)
MDIVMTQSPSSLSASVGDRVTITCRASQDVGTAVAWYQQKPGQSPKLLIHWASTRHTGV
PDRFSGSGSGTDFTLTISGLQSEDEADYFCQQYSNYPYTFGGGTKMEIKRGGGGSGGGG
SGGGGSGGGGSEVQLVESGGGLVKPGGSLKLSCAASGFTFSGYAMSWFRQAPGKRLEW
VATISSGGSYTYYLDSVKGRFTISRDNSKNTLYLQMSSLRSEDTAVYYCLASLITTDYFEY
WGQGTLVTVSS
>DAG5
(SEQ ID NO: 87)
MDIVMTQSPSSLSASVGGRVTITCRASQDVGTAVAWYQQKPGQSPKLLIYWASTRYTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGLTFSGYAMSWFRQAPEKRLEWV
ATISSGGSYTYYLDSVKGRLTISRDNSKNTLYLQMSSLRSEDTAVYYCLASLITTDYFEYW
GQGTLVTVSS
>DAG10
(SEQ ID NO: 88)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DBC8
(SEQ ID NO: 89)
MDIVMTQSPSSLSASVGDRVTITCRASQDVGTAVAWYQQKPGQSPKLLIYWASTRHTGV
PDRFSGSGSGADFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMNSLRSEDTAVYYCLASLITTDYFEY
WGQGTLVTVSS
>DBD5
(SEQ ID NO: 90)
MDIVMTQSPGSLSASVGDRVTITCRASQDVGTAVAWYQQKPGQSPKLLIYWASTRHTGV
PDRLSGSGSGTDFTLTISGLQSEDEADYFCQQYSNYPYTFGGGTKLEIKRGGGGSGGGGS
GGGGSGGGGSEVQLVESGGGLVKPGGSLKLSCAASGSTFSGYAMSWFRQAPEKRPEWV
ATISSGGSYTYYLDSVKGRFTISRDNSKNTLYLQMSSLRSEDTAVYYCLASLITTDYFEYW
GQGTLVTVSS
>DBD9
(SEQ ID NO: 91)
MDIVMTQSPSSLRASVGGRVTITCRASQDVGTAVAWYQQKPGQSPKLLIHWASTRHTGV
PDRFSGSGSGTDFTLTISGLQSEDEADYFCQQYSNYPYTFGGSTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSINTLYLQMSSLRSEDTAVYYCLASQFTTDYFEYW
GQGNLVTVSS
>DBD11
(SEQ ID NO: 92)
MDNVMTQSPSSLSASVGDRVTITCHASQDVGTAVAWYQQKPGRSPKLLIYWASTRHTGV
PDRFSGSGSGTDFTLTISGLQSVDGADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DBG3
(SEQ ID NO: 93)
MDIVMTQSPSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEINRGGGGSGGGGS
GGGGSGGGGSEVQLVESGGGLVKPGGSLKLSCAASGFTLSGYAMSWFRQAREKRLEWV
ATISSGGSYTYYLDSVKGRFTISRDNSKNTLYLQMSSLRSEDTAVYYCLASLITTDYFEYW
GQGTPVTVSS
>DBG4
(SEQ ID NO: 94)
MDIVMTQSPSSLSASVGDRVTITCRASQDVGTAVAWYQQKPGQSPKLLTYWASTRHTGV
PDRFSGSGSGTDFTLTISGLQSEDEADYSCQQYSNYPYTFGGGTKLEIKRGGGGSGGGGS
DGGGTGGGGSEVQLVESGGGLIKPGGSLKLSCAASGFTFSGYAMSWFRQAPEKRLEWV
ATISSGGSYTYYLDSVKGRFTISRDNSKNTLYLQMSSLRSEDTAVYYCLASLITTDYFEYW
GQGTLVTVSS
>DBG8
(SEQ ID NO: 95)
MDIVMTQSLSSLSASVGDRVTITCRASQDVGTAVAWYQQKPGQSPKLLIYWASTRHTGV
PDRFSGSGSGTDFTLTISGLQSEDEADYFCQQYSNYPYTFGGGTKLEIKRGGGGSGGGGS
GGGGSGGGGSEVQLVESGGGLVKPGGSLKLSCAASGFTFSGYAMSWFRQAPEKRLEWV
ATISSGGNYTYYLDSVKGRFTISRDNSKNILYLQMSSLRSEDTAVYYCLASLITTDYFEYW
GQGTLVTVSS
>DBG11
(SEQ ID NO: 96)
MDIVMTQSPSSLSASVGDRVTITCRASQDVGTAVAWYQQNPGQSPKLLIYWASTRHTGV
PDRFSGSGSGTDFTLTISSLQSEDEADYFCQQYSNYPYTFGGGTKLEIKRGGGGSGGGGS
GGGGSGGGGSEVQLVESGGGLVKPGGSLKLSCAASGFTFSGYTMSWFRQAPEKRLEWV
ATISSGGSYTYYLDSVKGRFTISRDNSKKTLYLQMSSLRSEDTAVYYCLASLITTDYFEYW
GQGTLVTVSS
ABC3317 Sequence
Light chain
(SEQ ID NO: 97)
DIVMTQSPSSLSASVGDRVTITCRASQDVGTAVAWYQQKPGQSPKLLIYWASTRHTGVPD
RFSGSGSGTDFTLTISGLQSEDEADYFCQQYSNYPYTFGGGTKLEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Heavy chain
(SEQ ID NO: 98)
EVQLVESGGGLVKPGGSLKLSCAASGFTFSGYAMSWFRQAPEKRLEWVATISSGGSYTY
YLDSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCGGGSRLIEDICLPRWGC
LWEDD
>ABC3320
Light chain
(SEQ ID NO: 3)
DIVMTQSPSSLSASVGDRVTITCRASQDVGTAVAWYQQKPGQSPKLLIYWASTRYTGVPD
RFSGSGSGTDFTLTISGLQSEDEADYFCQQYSNYPYTFGGGTKLEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Heavy chain
(SEQ ID NO: 4)
EVQLVESGGGLVKPGGSLKLSCAASGLTFSGYAMSWFRQAPEKRLEWVATISSGGSYTY
YLDSVKGRFTISRDNSKNTLYLQMSSLRSEDTAVYYCLASLITTDYFEYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSC
>hFM1
(SEQ ID NO: 99)
QAVLTQPSSVSGAPGQRVTISCTGSSSNIGAYYVHWYQQVPGTAPRLLIFDNDNRPSGVP
DRFSASKSGTSASLAIIGLQAEDEAEYYCQSVDYSLGDGVVFGGGTKLTVL
(SEQ ID NO: 100)
QVQLVESGPGLVKPSETLSLTCAVSGGSISSNNWWSWVRQTPGKGLEWIGEIYHSGSTNY
NPSLKSRVTISVDKSKNQFSLKLSSVTAADTAVYYCANLRGQWGQGTLVTVSS
>hFM2
(SEQ ID NO: 101)
QSVVTQPPSVSGAPGQRVTISCTGSSSNIGAGYDVHWYQQLPGTAPRLLISGNTNRPSGV
PDRFSGSKSGTSASLAITGLQAEDEADYYCQSYDSSLSGHVVFGGGTKLTVL
(SEQ ID NO: 102)
QVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARGKRGNFDYWGQGTLVTVSS
>hFM3
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM4
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM5
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM6
(SEQ ID NO: 129)
QAVLTQPSSVSGAPGQRVTISCTGSSSNIGAGYDVHWYQQLPGTAPKLLIYSNTNRPSGV
PDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEWIGEISHSGIPNY
NPSLASRVTISIDRSQNQFSLKLTSVTAADTAVYYCARGGGRFDFWGQGTLVTVSS
>hFM7
(SEQ ID NO: 137)
QAVLTQPSSVSGAPGQRVTISCTGTNSNIGAGYDVHWYQQFPGAAPKVLIYGNTNRPSG
VPGRFSASKSGTSASLAITGLQAEDEADYYCQTYDSSLSGTWVFGGGTKLTVL
(SEQ ID NO: 140)
QVQLQQWGPGLVKPSGTLSLTCAVSGVSISSSNWWSWVRQTPGKGLEWIGEISHSGSPN
YNPSLKSRVTISVDTSKNQFSLKLTSVTAADSAVYYCARGGGPIPHIWGQGTTVTVSS
>hFM8
(SEQ ID NO: 139)
QAVLTQPSSVSGAPGQRVTISCTGSSTNIGADFDVHWYQQLPGTAPKLLIHGDNNRPSGV
PDRFSGSKSGTSAYLAISGLQAEDEADYYCQTYDSRSSGSRVFGGGTKVTVL
(SEQ ID NO: 107)
QLQLQESGPGLVKPSGTLSLTCAVSGVSISSRNWWSWVRQTPGKGLEWIGEISHSGSTNY
NPSLKSRVTISVDTSKNQFSLKLSSVTAADTAVYYCARNAGDIWGQGTMVTVSS
>hFM9
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM11
(SEQ ID NO: 131)
QSVVTQPPSVSGAPGQRVTISCTGSSSNIGANYDVHWYQQLPGTAPKLLIYGNSNRPSGV
PDRFSGSKSGTSASLAITGLQAEDEADYYCQSYDSSLSGSSVFGGGTKLTVL
(SEQ ID NO: 132)
QVQLQQSGPGLVKPSGTLSLTCDVSGGSISSRNWWTWVRQPPGKGLEWIGEIYHSGSTN
YNPSLESRVAMSVDKSRYQFSLRLSSVTAADTAVYYCARRRDGYFDYWGQGTLVTVSS
>hFM12
(SEQ ID NO: 156)
QAVLTQPSSVSGAPGQRVAISCTGSSSNIAAGYDVQWYQQLPGTAPKLLIYGNSNRPSGV
PDRFSGSKSGTSASLAITGLQAEDEADYYCQSYDSSLSVLYVFGTGTKVTVL
(SEQ ID NO: 157)
QLQLQESGPGLVKPSGTLSLTCAVSGGSISSSNWWSWVRQPPGKGLEWIGEIYHSGSTNY
NPSLKSRVTISVDKSKNQFSLKLSSVTAADTAVYYCAKRPRNSGYLGAFDIWGQGTMVT
VSS
>hFM13
(SEQ ID NO: 158)
DVVMTQSPLSLAVTLGQPASISCRSSQSLLHSSGYKFLNWYLQKPGQSPQLLIYLGSNRA
SGVPDRFSGSGSGTDFTLKISRVEAEDVGVYYCMQALQSPTFGGGTKVEIK
(SEQ ID NO: 159)
EVQLVESGGGLVQPGGSLRLSCTASGFTFSSFSMNWVRQAPGKGPEWVSAISGSGGGTY
YADSVKGRFTISRDNAKNSLYLQMSSLRAEDTAVYYCARVRVPQAFDIWGQGTMVTVSS
>hFM14
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM15
(SEQ ID NO: 139)
QAVLTQPSSVSGAPGQRVTISCTGSSTNIGADFDVHWYQQLPGTAPKLLIHGDNNRPSGV
PDRFSGSKSGTSAYLAISGLQAEDEADYYCQTYDSRSSGSRVFGGGTKVTVL
(SEQ ID NO: 107)
QLQLQESGPGLVKPSGTLSLTCAVSGVSISSRNWWSWVRQTPGKGLEWIGEISHSGSTNY
NPSLKSRVTISVDTSKNQFSLKLSSVTAADTAVYYCARNAGDIWGQGTMVTVSS
>hFM16
(SEQ ID NO: 177)
QSVVTQPPSVSGAPGQRVTISCTGSSSNIGAGYDVHWYQQLPGTAPKLLIYGDTNRPSGV
PDRFSGSKSGTSASLAITGLQAEDEADYYCQSYDSSLSGSVVFGGGTKLTVL
(SEQ ID NO: 178)
QVQLQESGPGLVKPSGTLSLTCAVSGVSISTTNWWSWVRQPPGKGLEWIGEISHSGITNY
NPSLQSRVTISVDTSKNQFSLKLNSVTAADTAVYYCARVGEGAFKDLGQGTLVTVSS
>hFM18
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM19
(SEQ ID NO: 117)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKLTVL
(SEQ ID NO: 118)
QVQLVESGPGLVKPSGTLSLTCAVSGGSISSSDWWSWVRQPPGKGLEWIGEMRLSGTTN
YNPSLKSRVAISMDKSKNHFSLNLSSVTAADTAVYYCANADYYTHHYWGQGTLVTVSS
>hFM20
(SEQ ID NO: 119)
QAVLTQPSSVSGAPGQRVTISCTGSSSNIGAGYDVHWYQQLPGRAPKLLIYGNNQRPSGV
PDRFSGSTSGTSASLAITGPQAEDEADYYCQSYDSSLNGIWVFGGGTKLTVL
(SEQ ID NO: 120)
QVQLQESGPGLVKPSGTLSLTCAVSGVSISTTNWWSWVRQPPGKGLEWIGEINPSGSTNY
NPSLKSRVTMSLDTSKNQFSLKLRSVTAADTALYYCATRDYWGQGTLVTVSS
>hFM21
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM23
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM24
(SEQ ID NO: 131)
QSVVTQPPSVSGAPGQRVTISCTGSSSNIGANYDVHWYQQLPGTAPKLLIYGNSNRPSGV
PDRFSGSKSGTSASLAITGLQAEDEADYYCQSYDSSLSGSSVFGGGTKLTVL
(SEQ ID NO: 132)
QVQLQQSGPGLVKPSGTLSLTCDVSGGSISSRNWWTWVRQPPGKGLEWIGEIYHSGSTN
YNPSLESRVAMSVDKSRYQFSLRLSSVTAADTAVYYCARRRDGYFDYWGQGTLVTVSS
>hFM28
(SEQ ID NO: 108)
QSVLTQPPSVSAAPGQKVTISCSGSSSDIGNNFVSWYQQLPGTAPKRLIYDNSKRPSGIPE
RFSGSKSGTSATLGITGLQTGDEADYYCGAWDTSLSAYVFGTGTKVTVL
(SEQ ID NO: 109)
QVQLQQWGPGLVKPSETLSLTCAVYGGSFSGYYWSWIRQPPGKGLEWIGEINHSGSTNY
NPSLKSRVTISVDTSKNQFSLKLSSVTAADTAVYYCARYSNYRHYYYGMDVWGQGTLV
TVSS
>hFM29
(SEQ ID NO: 110)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM30
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM31
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM32
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM33
(SEQ ID NO: 169)
QSVLTQPPSVSAAPGQKVTISCSGSSSNIGNYHVSWYQQLPGTAPKLLIYDNNKRPSGIPD
RFSGSKSGTSATLDITGLQTGDEADYYCGTWDSSLSAVLFGRGTKLTVL
(SEQ ID NO: 170)
QLQLQESGPGLVKPSGTLSLTCAVSGGSISSGYYWSWIRQPPGKGLEWIGEINHSGSTNY
NPSLKSRVTISVDTSKNQFSLKLSSVTAADTAVYYCARLRGSGRWGQGTLVTVSS
>hFM34
(SEQ ID NO: 169)
QSVLTQPPSVSAAPGQKVTISCSGSSSNIGNYHVSWYQQLPGTAPKLLIYDNNKRPSGIPD
RFSGSKSGTSATLDITGLQTGDEADYYCGTWDSSLSAVLFGRGTKLTVL
(SEQ ID NO: 170)
QLQLQESGPGLVKPSGTLSLTCAVSGGSISSGYYWSWIRQPPGKGLEWIGEINHSGSTNY
NPSLKSRVTISVDTSKNQFSLKLSSVTAADTAVYYCARLRGSGRWGQGTLVTVSS
>hFM36
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM39
(SEQ ID NO: 145)
QAVLTQPSSVSGAPGQRVTISCTGSSSNIGAGYDVHWYQQLPGTAPKLLIYGNSNRPSGV
PDRFSGSKSGTSASLAITGLQAEDEADYYCQSYDSSLSGLYVFGTGTKVTVL
(SEQ ID NO: 146)
QVQLQESGPGLVKPSETLSLTCAVSGVSISTTNWWSWVRQPPGKGLEWIGEISHSGITNY
NPSLKSRVTISVDKAKNQFSLMLNSVTAADTAVYYCARGYGMDVWGQGTMVTVSS
>hFM40
(SEQ ID NO: 117)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKLTVL
(SEQ ID NO: 118)
QVQLVESGPGLVKPSGTLSLTCAVSGGSISSSDWWSWVRQPPGKGLEWIGEMRLSGTTN
YNPSLKSRVAISMDKSKNHFSLNLSSVTAADTAVYYCANADYYTHHYWGQGTLVTVSS
>hFM41
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM42
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM43
(SEQ ID NO: 117)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKLTVL
(SEQ ID NO: 118)
QVQLVESGPGLVKPSGTLSLTCAVSGGSISSSDWWSWVRQPPGKGLEWIGEMRLSGTTN
YNPSLKSRVAISMDKSKNHFSLNLSSVTAADTAVYYCANADYYTHHYWGQGTLVTVSS
>hFM44
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM45
(SEQ ID NO: 99)
QAVLTQPSSVSGAPGQRVTISCTGSSSNIGAYYVHWYQQVPGTAPRLLIFDNDNRPSGVP
DRFSASKSGTSASLAIIGLQAEDEAEYYCQSVDYSLGDGVVFGGGTKLTVL
(SEQ ID NO: 100)
QVQLVESGPGLVKPSETLSLTCAVSGGSISSNNWWSWVRQTPGKGLEWIGEIYHSGSTNY
NPSLKSRVTISVDKSKNQFSLKLSSVTAADTAVYYCANLRGQWGQGTLVTVSS
>hFM46
(SEQ ID NO: 148)
QSVLTQPPSVSGAPGQRVTISCTGSSSNIGAGYDVHWYQQLPGTAPKVLIYGNSNRPSGV
PDRFSGSKSGTSASLAITGLQAEDEADYYCQSYDSSLSGSVVFGGGTKLTVL
(SEQ ID NO: 147)
QVQLQQWGPGLVKPSGTLSLTCAVSGGSISSSNWWSWVRQFPGKGLEWIGEMSHTGST
NYNPSFKSRVTISVDKSKNQFSLKLSPVTAADTAVYYCARGSTGAFDVWGQGTMVTVSS
>hFM47
(SEQ ID NO: 134)
QAVLTQPSSVSGAPGQRVTISCTGSSSNIGAGYDVHWYQQLPGTAPKLLIYGNRNRPSGV
PDRFSGSKSGTSASLAITGLQAEDEADYYCQSYDSSLSGSKVFGTGTKVTVL
(SEQ ID NO: 105)
QLQLQESGPGLVKPSETLSLTCTVSGGSISSGNYWSWVRQSPEKGLEWIGEISHSGITNYN
PSLQSRVTIALDKSKNHFSLNVNSVTAADTAVYYCVGTLRTWFDYWGQGTLVTVSS
>hFM48
(SEQ ID NO: 142)
QSVLTQPPSVSAAPGQKVTISCSGSSSNIGNYYVSWYQQLPGTAPKLLIYDNNKRPSGIPD
RFSGSKSGTSATLGVTGLQTGDEADYYCGTWDSSLSAYVFGTGTKVTVQ
QVQLQESGPGLVKSSETLSLICAVSGGSISSNNWWSWVRQPPGKGLEWIGEIHHSGTTIN
YNPSLKSRVTISVDKSKNQFSLQLNSVTPEDTAVYFCARSASGAFDIWGQGTMVTVSS
>hFM49
(SEQ ID NO: 143)
QAVLTQPSSVSGAPGQRVTISCTGSSSNIGAGYDVQWYQQLPGTAPKLLIYADNNRPSGV
PDRFSGSKSGTSASLAITGLQAEDEADYYCQSYDSSLSGSKVFGTGTKVTVP
(SEQ ID NO: 144)
QVQLQESGPGLVELSGTLSLTCAVSGVSISSRNWWSWVRQTPGKGLEWIGEISHSGIPNY
NPSFVSRVTISIDRSQNQFSLKLTSMTAADTAVYYCARLYSGYGHGMDVWGQGTTVTVS
S
>hFM51
(SEQ ID NO: 145)
QAVLTQPSSVSGAPGQRVTISCTGSSSNIGAGYDVHWYQQLPGTAPKLLIYGNSNRPSGV
PDRFSGSKSGTSASLAITGLQAEDEADYYCQSYDSSLSGLYVFGTGTKVTVL
(SEQ ID NO: 179)
QLQLQESGPGLVKPSGTLSLNCAVSGVSISSTNWWSWVRQFPGKGLEWIGEINHSGTTN
YNPSLKSRVTISVDTSKNQFSLQLNSVTPEDTAVYFCAQHLTVWGQGTLVTVSS
>hFM52
(SEQ ID NO: 164)
QAVLTQPSSLSGAPGQRVTISCTGSSSNIGAGYDVHWYQQLPGTAPKLLISGNRNRPSGV
PDRFSGSKSGTSASLAITGLQAEDEADYYCQSYDSSLSGSFVFGTGTKVTVL
(SEQ ID NO: 165)
QLQLQESGPGLVEPSGTLSLTCAVSGVSISTRNWWSWVRQTPGKGLEWIGEISHSGIPNY
NPSFVSRVTISVDASKNQLSLKLTSVTVADTAVYYCSRKGVDAFDIWGQGTMVTVSS
>hFM53
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM57
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM58
(SEQ ID NO: 143)
QAVLTQPSSVSGAPGQRVTISCTGSSSNIGAGYDVQWYQQLPGTAPKLLIYADNNRPSGV
PDRFSGSKSGTSASLAITGLQAEDEADYYCQSYDSSLSGSKVFGTGTKVTVP
QVQLQESGPGLVEPSGTLSLTCAVSGVSISSRNWWSWVRQTPGKGLEWIGEISHSGIPNY
NPSFVSRVTISIDRSQNQFSLKLTSMTAADTAVYYCARLYSGYGHGMDVWGQGTTVTVS
S
>hFM59
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM60
QSVVTQPPSVSGAPGQRVTISCTGSSSNIGARYDVHWYQQLPGGAPKLLIHSNSNRPSGV
PDRFSGSKSGTSATLDITGLQTGDEADYYCGTWDSSLNSYVFGTGTKVTVL
QVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCASLRRGYWGQGTLVTVSS
>hFM62
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM64
(SEQ ID NO: 167)
QSVVTQPPSVSAAPGQKVTISCSGSSSNIGNYYVAWYQQVPGAAPKLLIYDTNKRPSGIP
DRFSGSKSGTSATLDITGLRTGDEADYYCGTWDSSLDTDVVFGGGTKLTVL
(SEQ ID NO: 168)
QVQLQESGPGLVKPSETLSLTCAVSGGSISSGSWWSWVRQAPGKGLEWIGEISHSGTTTY
NPSLKSRVTISLDKSTSHLSLSLKSVTAADTAVYYCARELGGGAYDIWGQGTIVTVSS
>hFM65
(SEQ ID NO: 131)
QSVVTQPPSVSGAPGQRVTISCTGSSSNIGANYDVHWYQQLPGTAPKLLIYGNSNRPSGV
PDRFSGSKSGTSASLAITGLQAEDEADYYCQSYDSSLSGSSVFGGGTKLTVL
(SEQ ID NO: 132)
QVQLQQSGPGLVKPSGTLSLTCDVSGGSISSRNWWTWVRQPPGKGLEWIGEIYHSGSTN
YNPSLESRVAMSVDKSRYQFSLRLSSVTAADTAVYYCARRRDGYFDYWGQGTLVTVSS
>hFM66
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM68
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM69
(SEQ ID NO: 140)
QAVLTQPSSVSGAPGQRVTISCTGTNSNIGAGYDVHWYQQFPGAAPKVLIYGNTNRPSG
VPGRFSASKSGTSASLAITGLQAEDEADYYCQTYDSSLSGTWVFGGGTKLTVL
QVQLQQWGPGLVKPSGTLSLTCAVSGVSISSSNWWSWVRQTPGKGLEWIGEISHSGSPN
YNPSLKSRVTISVDTSKNQFSLKLTSVTAADSAVYYCARGGGPIPHIWGQGTTVTVSS
>hFM70
(SEQ ID NO: 140)
QAVLTQPSSVSGAPGQRVTISCTGTNSNIGAGYDVHWYQQFPGAAPKVLIYGNTNRPSG
VPGRFSASKSGTSASLAITGLQAEDEADYYCQTYDSSLSGTWVFGGGTKLTVL
QVQLQQWGPGLVKPSGTLSLTCAVSGVSISSSNWWSWVRQTPGKGLEWIGEISHSGSPN
YNPSLKSRVTISVDTSKNQFSLKLTSVTAADSAVYYCARGGGPIPHIWGQGTTVTVSS
>hFM71
(SEQ ID NO: 142)
QSVLTQPPSVSAAPGQKVTISCSGSSSNIGNYYVSWYQQLPGTAPKLLIYDNNKRPSGIPD
RFSGSKSGTSATLGVTGLQTGDEADYYCGTWDSSLSAYVFGTGTKVTVQ
QVQLQESGPGLVKSSETLSLICAVSGGSISSNNWWSWVRQPPGKGLEWIGEIHHSGTTIN
YNPSLKSRVTISVDKSKNQFSLQLNSVTPEDTAVYFCARSASGAFDIWGQGTMVTVSS
>hFM72
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM73
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM74
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM75
(SEQ ID NO: 149)
QAVLTQPSSVSGAPGQRVTISCTGSSSNIGAGYDVNWHQQFPGTAPKVLIFGDNARPSGV
PDRFYASKSGTSASLTIIGVQSDDEADYYCSTWDDSLNAVVFGGGTTLTVL
QLQESGPGLVKPSGTLSLTCAVSGGSISSGNWWSWVRQPPGRGLEWIGEISHSGTINYNP
SLKSRVTISVDTSKNQFSLKLSSVTAADTAVYYCARRADGYFQPWGQGTLVTVSS
>hFM76
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM77
(SEQ ID NO: 129)
QAVLTQPSSVSGAPGQRVTISCTGSSSNIGAGYDVHWYQQLPGTAPKLLIYSNTNRPSGV
PDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEWIGEISHSGIPNY
NPSLASRVTISIDRSQNQFSLKLTSVTAADTAVYYCARGGGRFDFWGQGTLVTVSS
>hFM78
(SEQ ID NO: 162)
QAVLTQPSSVSGAPGQRVAISCTGSSSNIGAGYDVQWYQQLPGTAPKLLIYGNSNRPSGV
PDRFSGSKSGTSASLAITGLQAEDEADYYCQSYDSSLSGLRVFGGGTKLTVL
(SEQ ID NO: 163)
QLQLQESGPGLVKPSQTLSLTCTVSGASISSSNWWSWVRQTPGKGLEWIGEISHSGSPNY
NPSLKSRVTISVDTSKNQFSLKVSSVTAADTAVYYCAREPRYWGQGTLVTVSS
>hFM80
(SEQ ID NO: 164)
QAVLTQPSSLSGAPGQRVTISCTGSSSNIGAGYDVHWYQQLPGTAPKLLISGNRNRPSGV
PDRFSGSKSGTSASLAITGLQAEDEADYYCQSYDSSLSGSFVFGTGTKVTVL
(SEQ ID NO: 165)
QLQLQESGPGLVEPSGTLSLTCAVSGVSISTRNWWSWVRQTPGKGLEWIGEISHSGIPNY
NPSFVSRVTISVDASKNQLSLKLTSVTVADTAVYYCSRKGVDAFDIWGQGTMVTVSS
(SEQ ID NO: 166)
QLQLQESGPGLVEPSGTLSLTCAVSGVSISTRNWWSWVRQTPGKGLEWIGEISHSGIPNY
NPSFVSRVTISVDASKNQLSLKLTSVTVADTAVYYCSRKGVDAFDIWGQGTMVTVS
>hFM81
(SEQ ID NO: 167)
QSVVTQPPSVSAAPGQKVTISCSGSSSNIGNYYVAWYQQVPGAAPKLLIYDTNKRPSGIP
DRFSGSKSGTSATLDITGLRTGDEADYYCGTWDSSLDTDVVFGGGTKLTVL
(SEQ ID NO: 168)
QVQLQESGPGLVKPSETLSLTCAVSGGSISSGSWWSWVRQAPGKGLEWIGEISHSGTTTY
NPSLKSRVTISLDKSTSHLSLSLKSVTAADTAVYYCARELGGGAYDIWGQGTIVTVSS
>hFM82
(SEQ ID NO: 169)
QSVLTQPPSVSAAPGQKVTISCSGSSSNIGNYHVSWYQQLPGTAPKLLIYDNNKRPSGIPD
RFSGSKSGTSATLDITGLQTGDEADYYCGTWDSSLSAVLFGRGTKLTVL
(SEQ ID NO: 170)
QLQLQESGPGLVKPSGTLSLTCAVSGGSISSGYYWSWIRQPPGKGLEWIGEINHSGSTNY
NPSLKSRVTISVDTSKNQFSLKLSSVTAADTAVYYCARLRGSGRWGQGTLVTVSS
>hFM84
(SEQ ID NO: 171)
QAVLTQPSSVSGAPGQRLTISCTGSTSNIGAGYDVQWYQKLPGAAPKLLVYGNTNRPSG
VPDRFSGSKSGTSASLAITGLQAEDEGHYYCQSYDSSLSGWVVFGGGTKLTVL
(SEQ ID NO: 172)
QLVQSGPGLVKPSGILSLTCAVSGGSITSSNWWSWVRQPPGKGLEWIGEISHSGSTNYNPS
LKSRVTMSVDKSKNQLSLKLSSLTAADTAVYYCARGGSSLPIWGQGTTVTVSS
>hFM85
(SEQ ID NO: 173)
QAVLTQPSSVSGAPGQTVTISCTGSSSNIGADYDVHWYQQLPGTAPKLLIYGNNNRPSGV
PDRFSGSKSGASASLAITGLQADDEADYYCHSYDSTRSGLYIFGTGTRVIV
(SEQ ID NO: 174)
QLQLQESGPGLVKPSGTLSLTCAVSGVSISTTNWWSWVRQPPGKGLEWIGEISHSGITNY
NPSLQSRVTISVDKSKNQFSLKLSSVTAEDTAVYYCTTGSSGYWGQGTLVTVSS
>hFM87
(SEQ ID NO: 175)
QSVLTQPPSVSGAPGQRVTISCTGSSSNIGADYDVQWYQQLPGTAPKLLIYANNNRPSGV
PDRFSGSKSGTSASLAITGLQAEDEADYYCQSYDSSLSGLRVFGGGTKLTVL
(SEQ ID NO: 176)
QVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARSGRRSSWIDYWGQGTLVTVSS
>hFM88
(SEQ ID NO: 125)
QAVLTQPSSVSAAPGQKVTISCSGSDSNIGNYYVWWYQQLPGAAPKLLIYDNHRRPSGV
PDRFSGSKSGTSATLDITGLQTGDEADYYCGTWDGSLTGYVFGPGTKVTVL
(SEQ ID NO: 126)
QVQLQQWGAGLLKPSETLSLTCAVYGGSFSGYYWSWIRQPPGKGLEWIGEINHSGSTNY
NPSLKSRATISVDTSKNQFSLKLSSVTAADTAVYYCARGRVRGRWLPYYWGQGTLVTVS
S
>hFM90
(SEQ ID NO: 140)
QAVLTQPSSVSGAPGQRVTISCTGTNSNIGAGYDVHWYQQFPGAAPKVLIYGNTNRPSG
VPGRFSASKSGTSASLAITGLQAEDEADYYCQTYDSSLSGTWVFGGGTKLTVL
QVQLQQWGPGLVKPSGTLSLTCAVSGVSISSSNWWSWVRQTPGKGLEWIGEISHSGSPN
YNPSLKSRVTISVDTSKNQFSLKLTSVTAADSAVYYCARGGGPIPHIWGQGTTVTVSS
>hFM91
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM92
(SEQ ID NO: 160)
DIQLTQSPSSLSASVGDRVTITCRASQNINNFLNWYQQKPGNVPKLLIYAASSLQSGVPSR
FSGSGSGTDFTLTISSLQPEDFATYYCQQSYSPLSTFGQGTKVEIK
(SEQ ID NO: 161)
QVQLVQSGAEVKKPGSSVKVSCKASGYTFTGYYMHWVRQAPGQGLEWMGRINPNSGG
TNYAQKFQGRVTMTRDTSISTAYMELSRLRSDDTAVYYCARSVSVVTDAFDIWGQGTTV
TVSS
>hFM93
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM94
(SEQ ID NO: 153)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKVTVL
(SEQ ID NO: 154)
QVQLQESGPGLVKPSGTLSLTCDVSGGSISSNNWWSWVRQSPGKGLEWIGEIIHTGRTNY
NPSLTSRVTILIDKSKNQFSLKLTSVTPEDTALYYCARLRGPFDIWGQGTMVTVSS
>hFM96
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM97
(SEQ ID NO: 139)
QAVLTQPSSVSGAPGQRVTISCTGSSTNIGADFDVHWYQQLPGTAPKLLIHGDNNRPSGV
PDRFSGSKSGTSAYLAISGLQAEDEADYYCQTYDSRSSGSRVFGGGTKVTVL
(SEQ ID NO: 107)
QLQLQESGPGLVKPSGTLSLTCAVSGVSISSRNWWSWVRQTPGKGLEWIGEISHSGSTNY
NPSLKSRVTISVDTSKNQFSLKLSSVTAADTAVYYCARNAGDIWGQGTMVTVSS
>hFM98
(SEQ ID NO: 158)
DVVMTQSPLSLAVTLGQPASISCRSSQSLLHSSGYKFLNWYLQKPGQSPQLLIYLGSNRA
SGVPDRFSGSGSGTDFTLKISRVEAEDVGVYYCMQALQSPTFGGGTKVEIK
(SEQ ID NO: 159)
EVQLVESGGGLVQPGGSLRLSCTASGFTFSSFSMNWVRQAPGKGPEWVSAISGSGGGTY
YADSVKGRFTISRDNAKNSLYLQMSSLRAEDTAVYYCARVRVPQAFDIWGQGTMVTVSS
>hFM99
(SEQ ID NO: 121)
QAVLTQPSSVSGAPGQRVTISCTGSRSNIGANYDVHWYQQLPGTAPKLLIFNDNIRPSGVP
DRFSGSKSGTSASLAITGLQAEDEADYYCQSYDSSLSGSVVFGGGTKLTVL
(SEQ ID NO: 122)
QVQLQESGPGLVKPSGTLSLTCAVSGVSISTTNWWSWVRQPPGKGLEWIGEISHSGITNY
NPSLQSRVTIALDKSKNHFSLNVNSVTAADTAVYYCASYWLGNFDYWGQGTLVTVSS
>hFM100
(SEQ ID NO: 123)
QSVLTQPPSVSGAPGQRVTISCTGSSSNIGAGYEVHWYQQLPGRAPRLLIFDNNNRPSGV
PDRFSASKSGTSASLAITGLRAEDEGDYYCQSYDSKRTPPYVFGTGTRVTVL
(SEQ ID NO: 124)
QVQLQQSGAEVKKPGASVKVSCKASGYSFSKYGMSWVRQAPGQGLEWMGWINAGNG
DTKYSQKFQGRVTITRDTSASTAYMELSSLKYEDTAIYYCARRLSYYGMDVWGQGTTV
TVSS
>hFM101
(SEQ ID NO: 125)
QAVLTQPSSVSAAPGQKVTISCSGSDSNIGNYYVWWYQQLPGAAPKLLIYDNHRRPSGV
PDRFSGSKSGTSATLDITGLQTGDEADYYCGTWDGSLTGYVFGPGTKVTVL
(SEQ ID NO: 126)
QVQLQQWGAGLLKPSETLSLTCAVYGGSFSGYYWSWIRQPPGKGLEWIGEINHSGSTNY
NPSLKSRATISVDTSKNQFSLKLSSVTAADTAVYYCARGRVRGRWLPYYWGQGTLVTVS
S
>hFM102
(SEQ ID NO: 155)
QAVLTQPSSVSGAPGQRVIISCTGSSSNIGAGYDVHWYQQLPGTAPKLLIYGNTNRPSGVP
DRFSGSKSGTSASLAITGLQAEDEADYYCQSYDSSLSGNYVFGPGTKVTVL
(SEQ ID NO: 127)
QVQLQESGPGLVKPSETLSLTCAVSGVSISSHNWWSWVRQTPGKGLEWIGEISHSGITNY
NPSLKSRVTISVDTSKNQLSLKLSSVTAADTAVYYCARGSTGAFDVWGQGTMVTVSS
>hFM103
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM104
(SEQ ID NO: 112)
QSVVTQPPSVSGAPGQRVTISCTGSSSNIGAGYDVHWYQQLPGTAPKLLIYGNSNRPSGV
PDRFSGSKSGTSASLAITGLQAEDEADYYCQSYDSSLSGFWVFGGGTKLTVL
(SEQ ID NO: 113)
QVQLQESGPGLVKPSGTLSLTCAVSGGSISSSNWWSWVRQPPGKGLEWIGEISHSGSTNY
NPSLKSRVTISVDNSKNQFSLQLSSVTAADTAVYYCVSNASGRRGFAWGQGTLVTVSS
>hFM105
(SEQ ID NO: 149)
QAVLTQPSSVSGAPGQRVTISCTGSSSNIGAGYDVNWHQQFPGTAPKVLIFGDNARPSGV
PDRFYASKSGTSASLTIIGVQSDDEADYYCSTWDDSLNAVVFGGGTTLTVL
(SEQ ID NO: 150)
VQLQESGPGLVKPSGTLSLTCAVSGGSISSGNWWSWVRQPPGRGLEWIGEISHSGTINYN
PSLKSRVTISVDTSKNQFSLKLSSVTAADTAVYYCARRADGYFQPWGQGTLVTVSS
>hFM106
(SEQ ID NO: 148)
QSVLTQPPSVSGAPGQRVTISCTGSSSNIGAGYDVHWYQQLPGTAPKVLIYGNSNRPSGV
PDRFSGSKSGTSASLAITGLQAEDEADYYCQSYDSSLSGSVVFGGGTKLTVL
(SEQ ID NO: 147)
QVQLQQWGPGLVKPSGTLSLTCAVSGGSISSSNWWSWVRQFPGKGLEWIGEMSHTGST
NYNPSFKSRVTISVDKSKNQFSLKLSPVTAADTAVYYCARGSTGAFDVWGQGTMVTVSS
>hFM107
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM108
(SEQ ID NO: 151)
VLTQPPSVSGAPGQRVTISCTGSDSNIGAGYDVHWYQQYPGIAPKLLIYAHHKRPSGVPD
RFSGSTSGTSASLAITGLQAEDEADYYCQSYDSSLSGHYVFGTGTQVSVL
(SEQ ID NO: 152)
QVQLQESGPGLVKPSETLSLTCAVSGGSISSNNWWSWVRQTPGKGLEWIGEIYHSGNTN
YNPSLKSRVTISVDKSKNQFSLKLNSVTAADTAVYYCARGRQGAFDPWGQGTLVTVSS
>hFM109
(SEQ ID NO: 129)
QAVLTQPSSVSGAPGQRVTISCTGSSSNIGAGYDVHWYQQLPGTAPKLLIYSNTNRPSGV
PDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEWIGEISHSGIPNY
NPSLASRVTISIDRSQNQFSLKLTSVTAADTAVYYCARGGGRFDFWGQGTLVTVSS
>hFM110
(SEQ ID NO: 112)
QSVVTQPPSVSGAPGQRVTISCTGSSSNIGAGYDVHWYQQLPGTAPKLLIYGNSNRPSGV
PDRFSGSKSGTSASLAITGLQAEDEADYYCQSYDSSLSGFWVFGGGTKLTVL
(SEQ ID NO: 113)
QVQLQESGPGLVKPSGTLSLTCAVSGGSISSSNWWSWVRQPPGKGLEWIGEISHSGSTNY
NPSLKSRVTISVDNSKNQFSLQLSSVTAADTAVYYCVSNASGRRGFAWGQGTLVTVSS
>hFM112
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 114)
QVQLQESGPGLVELSGTLSLTCAVSGVSISSRNWWSWVRQTPGKGLEWIGEINHSGSTN
YNPSLKSRVTISVDTSKNQFSLKLSSVTAADTAVYYCARGGSDMDVWGKGTMVTVSS
>hFM113
(SEQ ID NO: 115)
QSVLTQPPSVSGAPGQRITISCTGSSSNIGAGYDVQWYQQVPGKAPKHLIYGNSNRPSGV
PDRFSGSKSGTSASLAITGLQAEDEADYFCQSYDSSLSGYVVFGGGTKLTVL
(SEQ ID NO: 116)
QVQLQESGPGLVKPSETLSLTCAVSGGSISSSNWWSWVRQPPGKGLEWIGEISHTGSPNY
NPSLASRVTISMDKSKNQFSLNLRSVTAADTSVYYCARYGRGAFDIWGQGTMVTVSS
>hFM114
(SEQ ID NO: 117)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKLTVL
(SEQ ID NO: 118)
QVQLVESGPGLVKPSGTLSLTCAVSGGSISSSDWWSWVRQPPGKGLEWIGEMRLSGTTN
YNPSLKSRVAISMDKSKNHFSLNLSSVTAADTAVYYCANADYYTHHYWGQGTLVTVSS
>hFM115
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM116
(SEQ ID NO: 153)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKVTVL
(SEQ ID NO: 154)
QVQLQESGPGLVKPSGTLSLTCDVSGGSISSNNWWSWVRQSPGKGLEWIGEIIHTGRTNY
NPSLTSRVTILIDKSKNQFSLKLTSVTPEDTALYYCARLRGPFDIWGQGTMVTVSS
>hFM117
(SEQ ID NO: 117)
QAVLTQPSSVSGAPGQRVTISCTGSSSNIGAGYDVQWYQQLPGTAPKLLIYADSNRPSGV
PDRFSGSKSGTSASLAITGLQAEDEADYYCQSYDSSLSGSKVFGTGTKLTVL
(SEQ ID NO: 118)
QVQLVESGPGLVKPSGTLSLTCAVSGGSISSSDWWSWVRQPPGKGLEWIGEMRLSGTTN
YNPSLKSRVAISMDKSKNHFSLNLSSVTAADTAVYYCANADYYTHHYWGQGTLVTVSS
>hFM118
(SEQ ID NO: 135)
VLTQPPSVSGAPGQRVTISCTGGSTNIGAGYDVHWYQQLPGTAPKLLIYGNNNRPSGVPD
RFSGSQSGASASLAITGLQADDEADYYCQSYDSRLDGSKVFGTGTKVTVL
(SEQ ID NO: 136)
QVQLQESGPGLVKPSETLSLTCAVSGVSISSHNWWSWVRQTPGKGLEWIGEMSHSGIPN
YNPSLESRVTISLDKSKNQFSLILRSVTAADTAMYYCVGGSGSYSYWGQGTLVTVSS
>hFM120
(SEQ ID NO: 112)
QSVVTQPPSVSGAPGQRVTISCTGSSSNIGAGYDVHWYQQLPGTAPKLLIYGNSNRPSGV
PDRFSGSKSGTSASLAITGLQAEDEADYYCQSYDSSLSGFWVFGGGTKLTVL
(SEQ ID NO: 113)
QVQLQESGPGLVKPSGTLSLTCAVSGGSISSSNWWSWVRQPPGKGLEWIGEISHSGSTNY
NPSLKSRVTISVDNSKNQFSLQLSSVTAADTAVYYCVSNASGRRGFAWGQGTLVTVSS
>hFM121
(SEQ ID NO: 128)
QSVLTQPPSVSAAPGQKVTISCSGSTSNVGNYYVAWYQKLPGEAPKVLIHDNNRRPSGIP
VRFSGSKSGTSATLGITGLQTGDEAEYYCAVWDSSLNGYVFGGGTKLTVL
(SEQ ID NO: 111)
QVQLQQWGAGLLKPSETLSLTCAVYGGSFSGYYWSWIRQPPGKGLEWIGEINHSGSTNY
NPSLKSRVTISVDTSKNQFSLKLSSLTAADTAVYYCARVNGGESDYWGQGTLVTVSS
>hFM122
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM123
(SEQ ID NO: 129)
QAVLTQPSSVSGAPGQRVTISCTGSSSNIGAGYDVHWYQQLPGTAPKLLIYSNTNRPSGV
PDRFSGSKSGTSASLAITGLQAEDEADYYCQSYDSSLSGLSVFGTGTKVTVL
(SEQ ID NO: 130)
QLQLQESGPGLVEPSGTLSLTCAVSGVSISSRNWWSWVRQTPGKGLEWIGEISHSGIPNY
NPSLASRVTISIDRSQNQFSLKLTSVTAADTAVYYCARGGGRFDFWGQGTLVTVSS
>hFM124
(SEQ ID NO: 131)
QSVVTQPPSVSGAPGQRVTISCTGSSSNIGANYDVHWYQQLPGTAPKLLIYGNSNRPSGV
PDRFSGSKSGTSASLAITGLQAEDEADYYCQSYDSSLSGSSVFGGGTKLTVL
(SEQ ID NO: 133)
QVQLQQSGPGLVKPFGRPCPLTCDVSGGSISSRNWWTWVRQPPGKGLEWIGEIYHSGST
NYNPSLESRVAMSVDKSRYQFSLRLSSVTAADTAVYYCARRRDGYFDYWGQGTLVTVSS
>hFM125
(SEQ ID NO: 131)
QSVVTQPPSVSGAPGQRVTISCTGSSSNIGANYDVHWYQQLPGTAPKLLIYGNSNRPSGV
PDRFSGSKSGTSASLAITGLQAEDEADYYCQSYDSSLSGSSVFGGGTKLTVL
(SEQ ID NO: 132)
QVQLQQSGPGLVKPSGTLSLTCDVSGGSISSRNWWTWVRQPPGKGLEWIGEIYHSGSTN
YNPSLESRVAMSVDKSRYQFSLRLSSVTAADTAVYYCARRRDGYFDYWGQGTLVTVSS
>hFM126
(SEQ ID NO: 103)
QSVLTQPPSVSGAPGQRVTISCTGSSSNIGAGYDVHWYQQLPGTAPKLLIYDNINRPSGVP
DRFSGSKSGASASLAITGLQAEDEADYYCQSYDSSLSGLRVFGTGTKVTVL
(SEQ ID NO: 104)
QVQLQESGPGLVEPSGTLSLTCAVSGVSISSRNWWSWVRQTPGKGLEWIGEINHSGSTNY
NPSLKSRVTISVDTSKNQFSLKLSSVTAADTAVYYCARGGSDMDVWGKGTMVTVSS
>hFM127
(SEQ ID NO: 134)
QAVLTQPSSVSGAPGQRVTISCTGSSSNIGAGYDVHWYQQLPGTAPKLLIYGNRNRPSGV
PDRFSGSKSGTSASLAITGLQAEDEADYYCQSYDSSLSGSKVFGTGTKVTVL
(SEQ ID NO: 105)
QLQLQESGPGLVKPSETLSLTCTVSGGSISSGNYWSWVRQSPEKGLEWIGEISHSGITNYN
PSLQSRVTIALDKSKNHFSLNVNSVTAADTAVYYCVGTLRTWFDYWGQGTLVTVSS
>hFM128
(SEQ ID NO: 106)
QAVLTQPSSVSGAPGQRVTISCTGSSTNIGAGFDVHWYQQLPGTAPKLLIYGDKNRPSGV
PDRFSGSKSGTSAYLAITGLQAEDEADYYCQTYDSRLSGSKVFGGGTKVTVL
(SEQ ID NO: 107)
QLQLQESGPGLVKPSGTLSLTCAVSGVSISSRNWWSWVRQTPGKGLEWIGEISHSGSTNY
NPSLKSRVTISVDTSKNQFSLKLSSVTAADTAVYYCARNAGDIWGQGTMVTVSS
>hFM130
(SEQ ID NO: 108)
QSVLTQPPSVSAAPGQKVTISCSGSSSDIGNNFVSWYQQLPGTAPKRLIYDNSKRPSGIPE
RFSGSKSGTSATLGITGLQTGDEADYYCGAWDTSLSAYVFGTGTKVTVL
(SEQ ID NO: 109)
QVQLQQWGPGLVKPSETLSLTCAVYGGSFSGYYWSWIRQPPGKGLEWIGEINHSGSTNY
NPSLKSRVTISVDTSKNQFSLKLSSVTAADTAVYYCARYSNYRHYYYGMDVWGQGTLV
TVSS.

22. A method comprising administering a binding partner of claim 21 to an individual who has received an antibody-drug conjugate(ADC) to thereby reduce non-target toxicity of the ADC.

23. A polynucleotide that encodes a binding partner of claim 21.

24. A polynucleotide that hybridizes to the polynucleotide of claim 23.
